SEQUENCE LISTING (33 sequences)

SEQ ID NO: 1 - DNA, 27937 bp, Homo sapiens
  source 1..27937: mol_type = genomic DNA
aggactcctccctcaccaaactctgcaggctttgaaatcaaagttctaaatgtctccccaggcaatcagaaaaggcaagacctggcaaataagaggttgtactaaccagtaacaaaatcacaaacaacatttgctcttcctcttccacagcagactccacaagtaggtgcaatgaaagagccctagatttggagccaaggccgtcaaatgccctcccagccattgtcactaatcacatatccacaagccagatcacttaatctctcaaagcctcaatgtccatatcttccaaatggggctaataattcaggttaactccatggaactttcatgagaaaagaccgtatgcaaaagcaactgaaaactgataaagcaccagatatgctagtaatgcattagtatcgtgaaataaacagggctcatttccaaaggtacaaagaccctgcaagtataaagacttcttcctaggtctagactttccatagaaatagctttcctacccactttctgatgccgagaattttgaaagttctttttcccttaggttgagatgtaaagggcaaatctgcatgggaaaagattgcttcaatttatcagtcatgggaacctggggtaaatgcattttcagagcatttattgaaaggagaatagtgggctactgaggtagaagagttgcaatctttatgtgggctaaaagaggcaaatccaggtgcctgggaaccttgtttatagttttgttctcctacaccggctcttttgtcagaattgcttaaaaaacaaacattgtttttgcaagacctcaccctagatgtctaaacttctaaaatccctcataatcaatttttctgacttttaatgcttatctagcaggtaacatgcattttaaattaatccttttatcaacacttcagctgaaaagctgaagtctaggagttgaaggaccctaaagtctcaaatcaaaaataaatacatcttttttcatctaggaagtatcaaaatgtgggtttatttaagtatttgggaggtagtatcttcttcagacacaaatagtgtgttccattttcttcaacactttgagcaattagtagacaaaccagttatttgattgtatttgaatacaattacttgactaagtcatataaatttccttcagtatgaaaaactaccacctcatggtgttttactattatttccctcaatttatactttgcataatgcattcctggtgcttcctcaatctacaagttcccttatcccaaaggaacaacttaatattagattggccatataaaatttccaccttcccaagtcaaaaatggttcatgattgactcaggttatgtgtagagccagatacctggattcaaagcccattcaggccatttactagatctaaaaccacaaatggttatataattttcctgaacctcagtttcctcatctgtaaaatgggcttaacaatagtgccaacctcagacagttgtaaaaattaaatgagataatgaacggaaagtacttagcacagtacctagcacgcagtaattacttagtacatgtcagcttaaaagagagaagggaatgaagttgatccatctatctgtattcccagtgcttatcacagtgccaatttgttatatacactaattaaaatttgcattggattggatagttttggtcttcaattctatcaaactgagccatgatgtagccataatcccgtgtgatgtttgtgtaagagtttaatgtttctattgttaaaagtaaaaccttgaacaaattaaatttagttgaatttatttgagcaaagaaaccattcatgaataagtcagcaccctgaattagtaaagatttagagatctccaatagaaatattggactgtcagtatttagagacaaaaatagcttgattggttacagctggcatttgccttacaggaacatgttttggcaatttgcagcctgcgattgactgaaagcatggctgctatgattggtcaagactcagctacttgttacatgaatacactctcaggttaggttgcggtttgtttatatattaggttaagtaccctacctacctaggcagttttgggccacattaaatttactttaacactatcgagttttatccattttcttagtggaataaggaacatgtggagactacctgagtactccaaaatttagagatcagaaagaggggagcacctgtggggagtggccagggatttggaggaaaaccatgggattgtcaggtctaagggcaaagtgaaaaaggtgctttgagaagaagggagagcagccttgccatttgctgctaagaggtctagtaagctgaaggttcaagagcaaacactgcatttggcaataaggaagccactggtgaccttgatgagagggaattccttggagcactgggggcaaaagccttagtggtcaattaaagacagaatgagaggtaagcttgtaaaacactgaaagcagacattttaaataaatttccctatagatgacagcatagatttggtggcactcagtaagacatatagagtcaagaggaggtatttaaagatgggattgtataagaacaataacacaagaagaatgtagtagaaagggaaaatagatcatgagaaagagaggggaaaactgcaggagcacagccttatgtgagaaacaagcagtacaaccagtgcacacatggtggtgctggcccgaggccagagcagggactcttcctctgcatagtgagaaggcagtgagaaggcaatgtgtggggtataaaggcaggcaatttgccagatttgctcatggaaaacggagttattcttttctgattgtttctattttctcagtgaattcagagtcaaagtgatcagctgagaatgagtagaaaggggctatggcagaagagaagttgtgactagccctcttgggatgggagagcaaatggactgggaaaaggtagtagagttaccaggccatggtgagggtccacttgagatgtatgtttgtaaatttaaagctaacaagttagtacaaagttgtgtttttcttcatctatgtttagctgctcagatgcaggcgcagagtagattaagagttgggtttaaccaaaattgaaggtttgctaggccagtccgacagagagcacaaattgcaaagtgtgtgcaagggattgcttatggtgaggcaccatggttaatctgatctggataaggagagaaaagaggtgatgaggtgtaacaaatgctaaaaacatagaggagtcagtggtggtctcagtgggagaaaaaggtgtgagggtactttaaacaggagcagggaatatagaggtggttggaaattggaatgcatgaaactaaaatgttggaggtggcatagacactgtaataacaaagtccacattatgactgtggagtgggaggctaaagtcatgtccatgaacacggacatggctgtgggagcttcagtgagaggctagggcaggtgaattatcttatgtggagattgacatctcacatccattgagatgactgatggtggagaggaaagtagtgatctatgtgcttaaattttatcaatgagggacagtggaacatgacagttagttgactgcaagaataagggcactgggtggcacagactgtagcatgtgctttaagacagcaggggttttgagaggaggaagaggagaaataccctggaagagatagtatgaagcaaagaggacacaggccttgttgtaagcatttaaagtgcattggattgacaagaagcaacaggtatttcagagaagagattggaaatgaagaatttcactgacgacagaactttgcaaaaggctgagtgtaagagcaggaggtgacacagcaaggtaggagattgagttagaacaccaacacaccaagatatatggagataagaatttaaagataagatggaagacctagatatcctggactgctgggggcacctagacattctctgtctgtaggaacatgaagtcaactatatcctcctaaagcaggaaccactccagctcttttctgtgttccctacacactcatattagacagggggttggtgatggggatgactgaatgaactaaggagtgaatgcatgactcacaaaaggtagaggaagattggtgctgtgggaggagtggagggagacattcatttggaaaatcagatggcaggagcctttttattgagtagtagaagagcaaagcagaaggaccagaatctggagtcaaaagacatggttgagttctcattctgcaacttcctagctgcaggtctttgggaaaatgactcctttatacataactctgacctcatctataaagtaaacctttcctccttaggagattgagcttcaaactgtcacctctttgaggctcctgtccctttactacaacactaatttcatcccacttggaaattgtgtagagctgtacaagtaaaagggtggacaataaacaggagaaatatagtaggttccacatactctaacgcccagcccttggcctatgtgccaacactcactcccaactccttgaaaagctactattaaaagagtttccctttggtttagaaagatgtttcttataatgcatagcacattaaaataataacaactaacaccacagagaggagtgtggaacacccagtgagagtaatacagataaggagccagggtctaaaacaagacacatagggttaccttgggatgtgatacaacaaggaacatcataacctcctgcttaggtagctgggcagaatcaaggctgccacagagcctgatggagtaggaggaacaatgcccagccattcccacacatgctcaggagcagggcagctatgtacatgttggagagatgctgtttgtctttgactcgcccgtgttctgagtgagccctttgacccagttttagaagcagactgagccacggtgagcagaggcggggcttagggaggcaggagtcttggggctttataaagtcctgccgggcaccactgggcatctctttcaaggtttctgctgggtttctgaactgctgggtttctgcttgctcctctggagatgcagcgtctgttgactccagtgaagcgcattctgcaactgacaagagcggtgcaggaaacctccctcacacctgctcgcctgctcccagtagcccaccaaaggtgagtcactttctgagaagcaccttgtaactagtaaaagatagtttttccctgctattggggaaaactcactagaatcccactcaaaatttggcaaggcttgtgcacagcagccttagacaagcaagttaactttaaagggtctcagttacctcatctctaaacagacaatccctttcagctgtagagtgagaagagcccaaacctctgacacatgctgtgtttgtgagcaatggcaacttttactctgccagctgcatgaagcagtagaaatatcagtaccaggccacagctttcctctctacaccaccattcccaccttcacccctagcctctgcctagaaccacaggaccttgtgccaactgcagtgttagtaaaaccagtgactttatatcactgcagcagaatcagaaatggactgaggatgagaagctgtgtttgccttgtgttccaattttatgaaaaggggaaatgtgtgtttatgtgtgtatgtgtacatgctctttgcaagaagaacatgcacactccttttctttgtaaatagtccctgaacatggctcaagtgcttatgttttccattgtcagcgatgatggtaacacagctatcgttagtgcctcaggctcccagccacctatgtgtttctgtctaatccccaaaccatccactacacattgggactagttctttatttccttacatttttactctatattctatgactactaaatatttagaaaaatgattttgacctagtgtctttccttgccaaatacccaaggaacctgggtgtatagatgtgcatggtagaggcaaatgcacatagctttcttatatttttcattatgctaccatcatctcactctccccatgcactgccaaaccctgcatgtgggttaaatgtcccagctcaggatttaacctgtttctatatttgtgaagaagagattgatgtgggtttcttgttttaatagcaatagttggccatcagccaaaagacatacatcaatcctccccaacattctgactcccttggttcaaactcttggaatcattcccatttcccttctggtatattcacagttaatcccattatgcatggcttgaactaatattgcttttcatgagtcaccttttctctatatgtctaatcgcctttaatccaacccacattggctctaactccaacctaaaagagactttcatctcagcatctgctttgctgtcttcaaaattcggtaagacttgtgccctccacttactgtatttctcacatattgtctccctgctcccctatacacctgcatctccagggttccttacttgttcagtcaccccctgccgtggccactgccccttcattcccctccagttcttcactggcagaagtctgtcatccatcaaggtttgcctcaaatgcggtctcttccacgaagcttctctgatcctccaacccactgaaatctctgcttcctttgaactcctgtagattttgctcacattcctttttgtgggcctgaccacattctgccttgaagttgggttatatgtgtgcttatcattcctcacactggtcaaggaggtctcaagaacctcaccctcttcttttctttgtccaacccctttgttcaaccctcacaaacccttcccagcacagtgcctgaagtgtagtaattaattttgaaacacaagggaaggaggcaagatggaatacagaagtaaaggtgtggtgcatgttcttgaagtgggcaacaccaggagaaaaatgatttaaaattacacaaagtgatcattctttagagaaagcacaagatgagaaggatactcttaacttcggtgggctgaagcttctggaagcctctccgtgttaattttcttcaaggctttataatccatttctagaaatagctccccaccaagacagctacaaaagttaccaactgacccattctaagcttcttcttgcaagctttgatttctaactgggaagaaagggagggagccagcccagagaagtcagagcgagaatgaggctgagagaaaggcagccaagctggcaggacaagcgctggcttaaccattagctcccgggtactggggaagctcctccgtaaatatttgagagtacaaactccagttatttggagggagtcaaataaatagggaagataaataaactccaaacctctcctgtcagatataatgtgtatttatcattctgcctcactatcttgtgatcatatgatccacttttgcctcacagctgtccttagaagtgaccttgctgctgggagaggctctagaattctaccagaggctcagaatcccaaagatgattgatagacacattcaatctgagttccagctcccgtagaatggagctaaatttataagcctggcacccagggcagtgaagggacagagtatttctaacacgtgagaaactatgaagttaccctgagtgcatcactttaccagtgtgtgccttggtttcactaactataaaatgaagaatgttgctaaagtgaacagaaggtataaagtacttttgtatgggagcagtacagagatcaccaagttcacctccagtatgctcccatacaaaagggaacacagattttcgccagggatattaagaatctgggttaaagagaagtgaattggtccagaaaagaaatagatcatctctcccttcttctgctgactccttccccttccttttttcctctgctctcgtttagaattgctctttctgctgtctgtgttccctgcatatttagctgtaaaatgtctgcttctttcactgggctgtgctctctttatgggcacaatgcatgtcttattcactgctgtgtatttggactagaactgtgttgggtgtgctcaataaacattggaaggccctatcagaaaaatcagctagcagaaaacttacttaaaagtaggaaaacagtgggtatgttcttgtgtagaaaaaagaaaggagaaagacatgtaattagaggtacacttttaaaatgagtaaagattgtataattatgccctataagggcttataacatgtagaagtaaagtatatgacaataatggttcaaaaggatgcagagagtaaataaagtcaacctaaagtttttgcagtgttccaaaagtaagataagtattaatttaagtaagattacaacaagccaattatgcatgttataatctttaaggtcaccagtaaaaggaaaagagggtataaaatgaataataaatatttgcttactctaaaaggatatgggaaaggaggaataaaagaacaaagaacaaatgagacaaatagaaacaaataaaaaaatagacttagttccggctgggcgtggtggctcacgcctgtaatcccagcactttggaagaccgagatcaggagatcgagaccatcctggctaacacggtgaaaccctgtctctactaaaaatacaaaaaattagctgggcgtggtggcaggtgcctgtagtcccagctactcaggaggctgaggcaggagaatggtgtgaacccaggaggcggagcttgcagtgagcagagatcacgccactgcactccagcttgggtgacagagtgagactccgggtgacagagtgagactccgtctcaaaaaagagaaaaaaaaagatttagttccaactatattagtaattacaacaaatataaatggatgaaatactcaaattaaaacaccactattgttagacttattaaaatttttttaaaggactaaaatatatataccgatcacaagagatgtatgttaaagataaagacgttaagaagttgaaagtaaaaagggacagaaaaagatgtaccatggaaacagtaagcaaaaagctagtgtagctatatcgacgtcaggaaaggaaactttatgccaagaatatcacaaagatgaaaagggatatttaataagtatagaagggtcaattcaatgaaaagataataacaatactaaatttgtagtcatctgataacatagcttcaaaatatagaaaattaattaaatgattgctatgttactgtcttttgaggaaattgtctacagaccattagtgggagtttgactgttatctccatcacaggttttctacagcctctgctgtccccctggccaaaacagatacttggccaaaggacgtgggcatcctggccctggaggtctacttcccagcccaatatgtggaccaaactgacctggagaagtataacaatgtggaagcaggaaagtatacagtgggcttgggccagacccgtatgggcttctgctcagtccaagaggacatcaactccctgtgcctgacggtggtgcaacggctgatggagcgcatacagctcccatgggactctgtgggcaggctggaagtaggcactgagaccatcattgacaagtccaaagctgtcaaaacagtgctcatggaactcttccaggattcaggcaatactgatattgagggcatagataccaccaatgcctgctacggtggtactgcctccctcttcaatgctgccaactggatggagtccagttcctgggatggtatgtacggccacgaaccttatgtaagaaaggtgctggaattggaggctgaatattaccagttttgcttttcagttccccaggtggcttcatctagtgaaggaaggacaatatattcacacagctgctgctatcatcccacaataaccacttagacttatatagctttacagttaggtagcatgttcacatagccattcatttaattcttacaacagcctaggaagtgtgtattataccagatttatagaagagaacatggaagatctgatagcttacacatagtgagtggcagaggcaaaaatgccaaaccacatctgacatatttcctattttaccgtacctgtttctcttaaacatgtcctaagtctctgagagattggtgatgttgaaagatgtatgcaagtttagatgttcgggaaaaaaacaccttcatagaaacaggcccagaaaaccacaagatagactgtgagtatttctactctttctcccttaggtggctccttgcatattgctttttgcttaacatattaacattaccttgtatcttacttatatcttctcccagtgctatatttgaggactaacccctgttgttacagcaagaaatgattcaagggaaacagtacagtatgagagcttgaagccatagctctatcaataatcattgataaattcctgaacctctttgagcctcagggttatttgcctatctgccttgcttaacttataagaggactgaataaaataattcatagaaatgtgaaattttcataaagatgtgaaaaaacagtatgttggcagtagttaagacactctatatttactaagtttgaaactaggattaaaaaccttagaaaccatgataagcattaattataaaattaatcaaaaagccttaatattggcagagtcctcagagatcatctaattcaatatcttttgctttagaaaaaagaggtcaagaggagtgtaacagtttatctctgtacatgcagcaagaccgtgcaattacaaaagttcattccaggcttttccaactgccctacctggctccatcattaacaattccactgacatgggatggtccagtctacatcatcaagtctgttcttaaagtgcctctcctacttgatacttgtattactacctctctagtaacccctaccaccattaccaccactgatatgtccaaccaattatttagttgaggagtagaaatgaaaaataaggggcattcaccagcctttaaccaaaaatcaaagagcctattcttgagagcattgtcagccttaagcatgccatttcaaatgcgtagattcttctgaggggctgggtattccacagatggggttgcaaatgcatcttttaaaaaaatgtggtatctaggtataaaagtaaaaatttaaaaaacaagttattgaaatgtgaatctttagtttgtatttaaaacaaaaacagctaagcttgagcctggacactcggactacataccctgcaggtgacagtaaccaccaggaccagaggatgccagtgtgaatgagaactctgcttctgacctagccagtcattcatctggggaccctcaggtgggagggagtggctctgagactcagggagttctgaatcactccagagaaaagtggaggggatgaggaaagagaagagtatttctggctcagattggctgggagtccccatgttttcttgtgtttttttttttaaatgaaaataattaaaatttatatttggaaaaaaacatacacatacacaaaagtatataaagcaaagaaagactcctcatttgacctgttaccacttcccaaaatttaacactgatggtttatatgtattcttccaatattttttctaagtacctgcaagtatacacatatctattccattttaaacattgtacaaaatattcctcatctcttaggtcttagaggtaattctgtatcaacatatgtaaggtctatctgattctttttaaaaccacaatattcttgatggatatgccaaattttatttaattaatcccatattgatggatatttagttttttagcaatgataaataaagttttaatgaacattgtacaatagctttgtatacttttggcattgtattgtaagaataaattcctagaagtggaatatcaggataggttgatttaaaagtttgataaaatgtgccaaattcttctccaaaatgttgtactaacttacattcctacaatgtatatattatcaaactttctaatctttgtcaatttaacaagtaaaattataatgtttttgatttgcgtttcttttactataagaaatcttgaatatttctatgttgtttattggcctttttttattatatagcttgcctttttttattttttatttatttatttttttagacagagtctcgatctgttgccaggctggagtgcagtggcggtgatctcagctcactgcaacctctgcctcccaggttcaagcgattcttctgtctcagcctcccgagtagctgggactacaggaccccaccaccacacccggctcattttttgtatttttagtagagatgggatttcaccgtgttagccaggatagtcttgatctcctgacctcacaatcctcctgcctcggcctccccaatcgctgggattacaggcgtgagccaccgtgcccggcctagcttgcctttttaatgaaacttttataaatgaagataaattgatttttgttgattgtaagtattgtaaatactcccccaatttgtcttttgactttgtttctgatagaaggctttgatttttagataatcaaatttactggccttttcctaaatggattctaaatacttttctatagtttctaaagttttcaaaatgtgtgcgtgtgtgtgcttatatacaggtagaaaaaagtattgttttcccttaattttatgtatataaaaattatatatacttaaatatatatttatatatattaaatataccaatttacttatactaatatatttatatatactaaatatatacttacatttatatatttatataaattatttgtatatttatatatatacacacacacacatgcacatagcattggggaagaaaacaatactttttcgttgatgttggagttgggattgttataattcttaagagaaggtccctggatttcagtgaatttgggttggagtcctgactctgaatccttaccctaccatttattagctatgtggtttttgggcaagtggcttaaattctttagccctcagtttcttcatctgtaggatggggataactatatctgctacatagatttatcatgaggattaaattatatagaaatgtggctcccaaagcagtgctgtgggtgaatactgggagcttcctcacaggtcagaatactaaaattactaccatatctcacccacaaacttgagtttttgggacagtacttcttacagatgaaagtggaacacataatagtcaagaccacaattatttattgaatactagtctgattatcataaagttagtgactacggatcatttactcaatataaactattttcacaatgaaagtagtgccacacaattcaaggcacgtggttcaggatccagtcagaactgggtttgaatatcaaaatccatattaactagctatgtgaccttacactagttactcagtctctcaggaaggcaatgtcttcacttgtgaatgtggatgttacctacctcattggattgtttcaagaattgtttaaggttaactagtgtcctactagtgttttaaatgttagtttccctccctgtcctttaccttctatgatttaggatataatttcaggatcatggtgtgctataaggagatgggtacaaacccaaacctgaattgtctccaaaagtgcgaattaacacatttttcactgaagtcagagacagaattctgaataaatgagcgttttacagagtgtcaggacactaaattttgactttacatttcaaatgtatcatgaattgcactagaacataagctccacaggactgggattttttattttgtttatcactctatatccaggacctagaattgtgcctggtacacagtaggcactcagtctactctagatttggtaatgatggtaaatatttcttgtttctctttacaggtcgttatgccatggtggtctgtggagacattgccgtctatcccagtggtaatgctcgtcccacaggtggggccggagctgtggctatgctgattgggcccaaggcccctctggccctggagcgaggtttgtagtaatccattaccaagaggctgtgcatggcatagccaagaacatagatcctaatcccacattggcacacctgctactcagggctgaggtatgcgtttgaggatggtattgcttgcctctaaaaagggctggtctatggagcagagggaggagaggagaaatgggagaggggaatccgcgaggcttcctctcttgcatcatcaggcattgggataacgatgcatggaatgagtggtgcagatgatggtgaggaatcttagggaactcttctggcaattgaagattaaaatatataactggatataaagtgaaagtctttcctttgagactgttggcttctattctaggttttgttaagcccatgtaggtgaggaaagggaaatatacatctcatttttgtaataccaacaacctgtccaactccttttgaatatgcaagggatgttgaatgggcttgaacttgggcatgggacacagataatgaccagaaacctcctttatatggttctctcatcttttgtgctcaaggtaggctgcattgtgtagtctctgaaacactttgtgtgcctttccagggctgaggggaacccatatggagaatgtgtatgacttctacaaaccaaatttggcctcggagtacccaatagtggatgggaagctttccatccagtgctacttgcgggccttggatcgatgttacacatcataccgtaaaaaaatccagaatcagtggaagcaaggtatgagattcagagggcagaaagtgggggctctatttacataggccaagggtttgtacccaaaggccatgagatggtcttttctctcctgccttgaaaataatgtcaagagaattgtttcctgtcctctttcttacactcttccctgggtctatgctaaaatccatttggaagtcattcaacttcaggtgtaaaattgcttctaacttgagctaaataaaagaaagtaaataatccagggcaaggcccccagtgtgaaaccaagggatgtcagccacctgagaagatggtgttaagaggctgggcagtcacattcgacagtggttggcatttgtttctggttaagtcaggcatggtttggctcttggtttgtggtttaccatcttttaaagtctcacgttgagaaatcatacctatattttctatatgctgaagtgttatcagtgatttttctcttcgtgatgctactgcaggttgattttattttcacctttagttttggaatttccctcctgagaaatatgtactgctttcataagcagaaaataagcaaataaatcttccttttaaaatacagaaaagcagggagtggtggctcacgcctgtaatcccagcaccttgggaagctgaggcaggaggattgcttgaacccaggaatttgagaccaatgtgggcaacaaagcaagaccctgtctctaaaaaaaaaaagtacaaaagttagccaggcatggtggcataagcctgtagtcccagctactcagaaggctgagatgggggaaattgcttgagaccaggagcccatgcagtaagctatgatcaagcaactgcctccagcctggactacagagtgaaacaaaccctgtctctaaaaacatataaataaataaaaataaaatacagttaaacctactttaaagacataaatagtattcttgcctgctcaggcatgcccagatgggcatccgcaaaagacagattgcagtgtgggagaaggcatggatgccttgggggtgtcataaagagctacctcttgtccctttctactgcagtgggtgggacaccacctgccagaggtgaacctcatgggcaagaagttgctttgggcctctctgcctcagtctgtcttctgtaattggttatttgctcctaactcctctgaattcttgtggcatttaaattttactccttatttgcatatgtaaggtgacagatgctgctttggatcccagcactaaaatgtaatatttcctaagggcagagattgcattgccctcttcttcagagtgagagagacagtctgtagagtagagtcagagacatctgaacctgaatccaaagccagccttttcaaagttggacagatgacaatgttttgtagaccggttcctcctctggcaaatgaagaaaattatataacacaaggttgatttgagccaagtatcatagaggctggtaatagtagatacaaaggctttgtttctttcccttctttccttattcgtagagattgcttagtaagtgcatgtaaaatgaataaataaagctcatatgtgtttgcaggaggtgggaagtagttccctgggaggcctggagaaactcggcacagttaaatctcagggaggatatctaaatggctcgcccctcatgccccatccttgccttcacgcttcctcttccagctggcagcgatcgacccttcacccttgacgatttacagtacatgatctttcatacacccttttgcaagatggtccagaagtctctggctcgcctgatgttcaatgacttcctgtcagccagcagtgacacacaaaccagcttatataaggggctggaggctttcgggtgagttctcttcttggggagcctagaggctggtgaggtgtgagcaagaaggaggcttcttcatgccttaagtctagaccaccagcacccctgtgggggacaaatggcaatcctccagcagaacaggaacaatcccaggtccttccacggggtagtgggttattgtctgggtagggccctccatgagttattgcagggaaacatgggggatttggcagcactgcaggatcaaggggcagtaagaaactacagaggataaagaaagaaagagagaaagggagaaagagaggaagggagaaagagagtagctaaatcattcagtcaataaacattttctgaacatgttatgtgctagacatcgtattaacctctcaggatactaaaatgaatgtgactccatggtccctgccctagagcatctcacagcctatacagacacaaacacacagaagcaaatgatcacactacagggtagcaatttgagaagtgtcaggtcccattctcatttgccattgtcttaattcatgtcctgcttttgcttttctcccatctataaaatggggatgttccagctcatccccttagatgtgaaaaagcagaaagaatgctgtttattgattcactacactaatacactaatatttacaaagaaatgtcttcaatacagtttccactgggaaaggaatctttccctttcttcttggtacctgtttatttcaaattttggtcaattttatcaacagtagaataggctaccaagtgtagcccctgttactaactagtactcctaaccctgccactaactaaaacatcaaaattagcacaaacactgcttgtaagaccagccctatcgaaacaaaaagtataacatataccaaagatactagcttaatatctttaatatataaagatattatcaataataaaataaataccctaatagaaaaatgagcaaaggatatgaacagaaaattttctcaaagaagacatgtatatgatcacattttaaatatgcatattcattaataaaaagtttactcaagttaccattttctctagatagtctttttaaaatgtgaatacccagaattgtcaagcctgtgggaaaatgggcagtagtggatgcgtaaatggatcaggtgttttgaggagtaacaggagagcatgaagccaaagccttaaagatgtgcaaactttgggctcagtaatcccatgtgtttaaaagaacacctacctattcgctgtagtgttttaactagtgaggaacaggagcaggaagatgggttaaagtgcggtacatcctgtcatggaccattcttcagcctttacaaataatgttatagaatgtcatggaaaaaaaatatatatatatatacacacacacactaagttaagaaagtatgctaaccacaacacatagcatgattttctttctaattttctagtaagctctataaaattagggatggattccaccagaaaaataagccctaagtactctctctgaatggtaaggccattagtggtatgttctcctctgtactgttctgtatttccaaatattgtaggaaaaacatgcgccccaaagtcctctccagaagctgttacttttcccccttgctccctgcctcccgtcccctggcctctcacatggctacctctggctacctcacagggggctaaagctggaagacacctacaccaacaaggacctggataaagcacttctaaaggcctctcaggacatgttcgacaagaaaaccaaggcttccctttacctctccactcacaatgggaacatgtacacctcatccctgtacgggtgcctggcctcgcttctgtcccagtgagtactgcatctggctccatgtcctccatgcacaccctcagcctccgcccccgtgggctgcagggtcaacaaagttgggtttctcttttggctcagaaatttaaaagaaaggaaggggcctggtgtagtggctcatgcctgtaatctcagcatttggggaggtttaggcgggcagatcgcctgaacctaggagttcgagacccgcctgggcaacgtggtgaaacctcatctctacaaaaattagctgagcatggttgtgtgcacgtgtggtcccagctgctcgggaggctgaagtgggaggatggtgtgagcccaggagtggaaggttgcagtgagccatgattgtgtcattggactccaacctggatgacagaatgagatcctgtcataaataaataaataaatataaaagaaaggaaaggagggagaaggcaggaaaaggaaggaagatgaaagaaactcgtaccaaaggtgtatgtataggcagatttacagtctgtatcagacagtggtctccaaagtgaagtacatgatgtcaagggatgggcaagatctgtttgggcacatcaagaaaacagtagctttggtatgcatatttttgtctcatttatttaaaatctctatacttagtagagcatggtggttaaatgggtctgactttagagcccacaacctgggttcaaatttttttaaccaattattagggttgactttggataatacttaacctcaatgcacctcaccttccccaactgtagcatgtgtgcaatcacaatacctgtgttctactgtttttatgagcattaagtatctaaaacaattaaaatagcagtgcttagcaggtgctcaaatgttggatgttatttctattcattttctgttttgtgggttttataaggaagtactgcatctaacataagaaagggctcatgaagtggctcatgcctataatcctagcactttggaaggctcaggcaggaggatctcttgagctcaggagtttgagaccagccttgggaacagagggaggccccatctctacaaaatttttttaaacaattagccatggatgttcacggtggctcatgcctgtaataccaacactttgggaggccaaggtgggaagatcacctgaggtcaggagtttgagaacagcctggccaacatggcaaaaccccttctctactaaaaatacaaacatcagctgggcatggtggtacgtgcctgtagtcccagcaactcgagaggctgaggcatgagaattgcttgaacccgggaggcagaagttgcagtgagctgagatcgagctactgcactatagcttgggtgacagagtgagactctgtctcaaaaaaaaaaaaaaaattagctgggtgtggcagcttgcacctgtagtcccagctactcaggatcctgaatcctgaggtgggaggatcacttgagcccaggaggtaaaggctgcagtgagccatgatcacgccactgcattccgggcactccaggctgggcaacagagcaagactctgccaaaaaaagaaaaaaaaaacgggcaggaaaaagtgcttatgggtgaacttgatcaaattattactcacaggggatgatcaaaaagttatgactgctgaaccattaccaatcaacatgggagcctgaagggtgagtccagtggtctgatctccatctggagacaccttcagaatgcactgaatttaccctgtcctcatgagaggggagaagctctatgtacaccaaaaattatcttgtgttttctctgccttatatatcttggatattagctgctttccttttggcaaggtttcctacacaaaggcctgtccctggggtctaccagaagtccctctttatgtagggtgcctggaacccatttctagttgcatgaggtagacagggagaagatcgggatgataggctgttgttctatttgaagtgcagaatataatatatatatacatatatgtgtgtgtgtgtgtgtgtgtgtgtgtgtgtgtgtgttttatttgatttctttccccacagccactctgcccaagaactggctggctccaggattggtgccttctcttatggctctggtttagcagcaagtttcttttcatttcgagtatcccaggatgctgctccaggtgagtgtcatctttctagtaggccttcctgacaagattcatctggtagaataaccatcttcttccccaccattactgaggctgccatcttgacagagttacgttattattaatagcaaagtaaatcactgaagggatttaagcatggagtaagtttgtttaatttatgtgtttaaagcacttatttggctactacttagagactagattgaaaaggaacaaagctggatatggggaaaccacttagattgttccagtaactagttcaggcaagaggtaatggtggtttgattgcaactgattaaagagaagttgatggatttgagatacctaataagaatttattgattattttgtgattgatgtgattaaggacatgcatttaagtactatgtggcatacaccttgaccaaatcagtgtgtctgcctgcatgttttgctaacaagtatgcttgcttatcatttcttggtattctaagccacacacaccacacgttcctccagggtgtaacctcccacagaacctggctctctgttgaactcgtgattggcaatagtgataatgacaatgaaaaaggtgtaacaatcttgcttttgcttcccaggctctcccctggacaagttggtgtccagcacatcagacctgccaaaacgcctagcctcccgaaagtgtgtgtctcctgaggagttcacagaaataatgaaccaaagagagcaattctaccataagggtaagaaaaaagtcaggaagagaggaagagagaccccattccagtagctgggagccagggatttctttggaaatctagaatttagtagtccagggtcaagacttttacgagatatggttgggagaagatttgctagaagatctgttgtccaaaggggcaagaagtgggtggggaaacagaagatagagttgggaagagggaggcaggatgcagcttcccagtatagaatatagctaaacacccagaatgtgtagtcccatggaagccagaagtatagtctttgaaaataccatctgcaacagttgaaagagtacagactttagagctagatatccaaatctaaccctgagctgtgccactcactagctgtttatctttggaaaaatggttgaacttttctcagttgtcttatttctaaaatcataccgattttgcaggatttccaaacaaattaaatgaattactctatataaatatgttatcgacaaatattactgtcccctccaaattgccctctttctccaccaaacataaaaacaaaaaacaaaatattgctccaaaagcaacaaatgaaaggaaaatgaaacccaaaggtaatactagagtgattagttggtggttttaaaaccatagtaatacacagttttaccatgatttctacaggttttatatatattctcaagcaaaacttgggatgcatgttgttttgcagcatggtctcaaaaggagacagaatatacggaattggaaatgttccagaaaacctagacctagtggtcattgatctcttctggaccagtggatatgttatagcaaagaaagacaatgaaaataaaaatggagcagggcacagtggctcacgcctgtaatgctagtcctttgggaggcagaggcaggtggatcacttgaggccaggagtttgagaccagcctggccaacatggtgaaaacccatctctactaaaaatataaaaattataaaaatatgaatataataaaaaaaataaaattatgtaaaaattagccgagtgtggtggcacacacctctaatctcagctactcaggaggctgaggagaattacttgaacccaggaggcagaggatgcagtgaactgagatcacaccaccacactctagcctgggtgacacagaaagactctgtctcaaaacaaaaaaaaaaaaaagaagaaaagaaaaataggacctctgagacaaacgttaacggacaaagcactgaaatactgcaatgaatcagaaccagaaaatttagagtttagaaggacgtgtctgttaggaaacaggaagctgggaattacgtctcaaagtaggaactattggcaaaaggatgggatgaagatttcaatggaggaaggctatgtttactgtaggaaaatgttgtactcttataataaaagtcttaatagacttttattaaggccttaagtgctagattcaagatggctgcccctcttgttctgtgggtccagtgttctatttggtggactaagggtgaccttgcagccccttacagcccagccaagagagcttcactgtgaaggggcagacatcttcattactattttctcttccaaaaactcatataactctttgtgagtactgcctcttctcctcattccacagtgaatttctccccacctggtgacacaaacagccttttcccaggtacttggtacctggagcgagtggacgagcagcatcgccgaaagtatgcccggcgtcccgtctaaaggtggtgagtgagagtttgcagagttggtggcataaaaccctaatgtcttcctctgagtaacaacacagagagagaaggtggggacaggtgcagggagaagaaagtttaatggaagaggattggggtgacaggagaaatgggagaattatctgtggaatttttaaaaggaaaagcaagtattcagaataggaatcttgtagtttgggaacattaaccaggccagggagggttcacagctttcaaactaatcagaagtggggatttgtaccataaagaccaattaaaactcttggggctctttgccttggaaaggcaaaagctgggggagaaacatgttctgaaatcttgaatgtgaaaaataggagctggatttgtttacctgatctgctgaagataggaagctctcctagaagcttgacagattagcattcagagcatccgttgagtgaacaggctgtgaacctgaacctatagaaatcattactccagggggatgagatcaacagatctgatgagcaacagaacaaccaagatgaacagccccaaaacctcagaaatggtacacaccaatgtgtgggagacagattcataaggaatggggcggttgaagattctgttaaagccagatacttctgctggagggagttttaggctaagggtcatgtaacaattcttatatcatgggattccttctggggagaagcaatgaggttcaggaaattcgtggacacaaggatagggagaagagagcaaggtgaaagaggattgcggtgacaggagaaatgggagatattctttatgatcgtttttaaaggaaagcaaacattcaaaaataagaatcttatatgaacccaggtagctgccttcagttgaccaaataggtaggataagcagaatgatagagtgagaagagatttattttacaacccataaattttaattagtgcagtctccatgctcaagtttttaagattttcccctccttttggtagatggagagggaagaagaaaaaggtgtgccgaggcagggaaggagcagaggaagggaaggaagaagtcagtgggtggcagagatgcacagatacagccacctgagaggaagcagaggtgcgggtggaggggccctgggttcattccttaccgctgggatattggcaggtgctaggctgttgcagcccagatgttgttagggctaggagaggtggacaagtgggctgagggccgcaggatgcctttgagaggacgagctcagttagcagccctgaagactgtggtactgcccgggagcctgtgtgcatgttggaaatacggttcttaagggcaggtcagtagcaaagaggggctgttaaatgtgtcaacttagttcattcatcagaagaagagtgggagaaatagggagggaggggggaaagggagagagagaggttggggagagagtcagcgggagggggagagagaaagagaaatttggaatttttaaaggagaatttccacgtcagcctccctccctctcatggtagacaagcttcttgcaagtgcttaggcagaattatacctgaaaaaaaaagctggaactcttgaccttttctcatgttgattattaatatgagcagtgaacttccaacaatgagattttagcagaaatgaagggctgctgtcagtgcagtgctcatggtggagctctacaggtctctgcagcgccctagcctgcctctcctgctctcctatcacaggcagatgtgcgacggggaccctgcctacccccagccttggctccagtagcattgggcacagatccctcaggtgtccaggcttggcacagggtgcatagtgggagcaccctcaggatgcagttaggggagcccctctgcacagccacacctcgggcaagaagcaggtactgggggcagggtgcccaagaggagacccatgattgaatgactttttgtttatttaagttctgcagatccatggaaagcttcctgggaaacgtatgctagcagagcttctccccgtgaatcatatttttaagatcccactcttagctggtaaatgaatttgaatcgacatagtagccccataagcatcagccctgtagagtgaggagccatctctagcgggcccttcattcctctccatgctgcaatcactgtcctgggcttatggtgctatggactaggggtcctttgtgaaagagcaagatggagcaatggagagaagacctcttcctgaatcactggactccagaaatgtgcatgcagatcagctgttgccttcaagatccagataaactttcctgtcatgtgttagaactttattattattaatattgttaaacttctgtgctgttcctgtgaatctccaaattttgtaccttgttctaagctaatatatagcaattaaaaagagagaaagaggaaatgattcctgcgtttcttggaacccagaatacaaacccagcctaacatgcagcaagcctgctagaccttgtgggtcagagggctgggtccttgcctcacaggctgcctctgtccccttgcaattccattctatttctgccacatgccaagtgctatgacaggtacaaggcaaataagaacggtagaacacagcttcccccagcccacttccctgttctaaagacaccacatagacagagagcagcagacaggggccagcaggagctgtagttcagatcttcttggtcattccttgccgctgttatttgaacaaataaacacagcgcaaaggttaacaagtttttgccttctatagccaaaaataaaaaaataaataaattttgatgcctggcaggaaattattccattacaggatctttcccccttgggggagggcactgcttcttctagggtcctcttataaaatagcaatggttcaggcagatggggattgagctgaggacgggagtgggaggagagggaaagtatcagggtgttgtcatcacttccttttagaaagtttcctcagtcacccccatgaggaaagggcaccttggaaaagagagaggatgctttccattggcggggagcagagctggtgggggcaggggaggaggaggggaggaggaggaggaggagaagcaggggaggcttaaggctcccttaagcctcagggagcgcttaagaatggccccacaggaatgagaagctgggtctgttcccttcactgttttgctcaaggctgttcatgtcacaacaaatcccagataagccccaatttgctcagagaatccagcattagctgactgccttcccaggcctctctcaaggtgcctgcaaaactctactcatcacaccagctgcagccgctgcttagcagcccctctttgctaccctcttgctgcctgcacctcctcagcaagatgtttaggggccctcaacctggttggcatccctagcagaacaacatgtgcctttcggtatctgtgtgcaggggagaaaacccagcactaaccttagctctggagacaagaggcctcgggcctggccttctatccacacagaagctcactgtgcagtgttggtgctgaaactctctccatcagcctcagtcagcctcagcaaccagaacttcccatacttcctgcatcagaggccaggcctgtctccactagggaggcatttgagcacaaatggaatgatattaaacattcgacaaccaggttgtcaagggctgaccaattgaatggacactgcccacagcccacacaccagctgggcatcagcactggctccctccaacttccttattcaccaacttttatactgagcccgaggccttcctctggcagctctgggacactgatgcctgcctgctctgaacaaagccctctcccccatgtaaggtcagcacacgagggaatgagttgccaatggctcagtcaacattttcaccctaaagtctacagataccatacaaataaagactttccctgtgggcaaaaattcacacagggtgacctagggcaggagagaggacggcagattgggcaagtgttgggctatgatacactcattcaaacgggaatactcaacatgtgatgttaaaactgatgcaaaagatggccccgccactgaccatgagacaagcccaagctctagggggacacactgatcacaacttcaggagtcagcacattgaggcagattctgtgcgtggcccagcttttgccctgcctccaccctgagctcacagccagccttctgctgtgtgtgcacaagaatgaacttctactctaaaggggcagtgaagagatgccacatgccacaaagaacatgagggagtccatggcaccctccctgtagccctagctggatttttcaaaaatttcattgtatatatttgagggatagaacatgacgttgtaagatatatatatgtagtaaaatggttactgtaacggaacaaattaacatattcattattttacaaagttacccatcccccgccaccatggcaagagcagctgcaatctactaatttaggaaaaatcctcagtacaatacactgttattaactatagtcctcaggttgtacatcagatcttttgacttactcaccctatgtattttctactttacattctttgacctgtatctccctagacacccccctcaactacttttctagttcctatgtcaatatatttgacctcttttttggggggggattccacatataaatgagtaagtgcaataattttctttttgtgtctggcctatttacttagtcatcagggaaatgcaaatcaaaaccacggtgagataccacctcacacctgtta

SEQ ID NO: 2 - DNA, 23 bp, synthetic construct
  source 1..23: mol_type = other DNA
atacatggccaaaagatgtgggc

SEQ ID NO: 3 - DNA, 23 bp, synthetic construct
  source 1..23: mol_type = other DNA
gcacgacgggacaccgggcatac

SEQ ID NO: 4 - DNA, 23 bp, synthetic construct
  source 1..23: mol_type = other DNA
catcactgccacccagaagactg

SEQ ID NO: 5 - DNA, 23 bp, synthetic construct
  source 1..23: mol_type = other DNA
atgccagtgagcttcccgttcag

SEQ ID NO: 6 - DNA, 23 bp, synthetic construct
  source 1..23: mol_type = other DNA
cctgcagaaggagctagaacagt

SEQ ID NO: 7 - DNA, 22 bp, synthetic construct
  source 1..22: mol_type = other DNA
tgttcttaaggctgagctgcaa

SEQ ID NO: 8 - DNA, 23 bp, synthetic construct
  source 1..23: mol_type = other DNA
gcacatgaacggctggagcaacg

SEQ ID NO: 9 - DNA, 25 bp, synthetic construct
  source 1..25: mol_type = other DNA
tgctgcgagtaggacatgctgtagg

SEQ ID NO: 10 - DNA, 21 bp, synthetic construct
  source 1..21: mol_type = other DNA
gaaattcgcccgctccgatga

SEQ ID NO: 11 - DNA, 21 bp, synthetic construct
  source 1..21: mol_type = other DNA
ctgtgtgtttgcggtagtgcc

SEQ ID NO: 12 - DNA, 20 bp, synthetic construct
  source 1..20: mol_type = other DNA
gcccccaaggtagtgatcct

SEQ ID NO: 13 - DNA, 20 bp, synthetic construct
  source 1..20: mol_type = other DNA
gtcctcgtctgcttgaatgg

SEQ ID NO: 14 - DNA, 22 bp, synthetic construct
  source 1..22: mol_type = other DNA
cgaaaggacaagagaaataagg

SEQ ID NO: 15 - DNA, 24 bp, synthetic construct
  source 1..24: mol_type = other DNA
ctgtaaagttttaagttttatgcg

SEQ ID NO: 16 - DNA, 20 bp, synthetic construct
  source 1..20: mol_type = other DNA
agtctacccacctctagccg

SEQ ID NO: 17 - DNA, 20 bp, synthetic construct
  source 1..20: mol_type = other DNA
tgtgttatggctgggggttt

SEQ ID NO: 18 - DNA, 21 bp, synthetic construct
  source 1..21: mol_type = other DNA
tccacacaccaaaaggacgaa

SEQ ID NO: 19 - DNA, 21 bp, synthetic construct
  source 1..21: mol_type = other DNA
ccagctcatagtggaatggct

SEQ ID NO: 20 - DNA, 22 bp, synthetic construct
  source 1..22: mol_type = other DNA
catcacaaacattcccactggc

SEQ ID NO: 21 - DNA, 25 bp, synthetic construct
  source 1..25: mol_type = other DNA
tgaggcaaatagattttcgttcatt

SEQ ID NO: 22 - DNA, 20 bp, synthetic construct
  source 1..20: mol_type = other DNA
gacgaaatcaacaaccccgt

SEQ ID NO: 23 - DNA, 21 bp, synthetic construct
  source 1..21: mol_type = other DNA
tagcagtcgtagttcaccagg

SEQ ID NO: 24 - DNA, 20 bp, synthetic construct
  source 1..20: mol_type = other DNA
caagggatcccactgcacat

SEQ ID NO: 25 - DNA, 20 bp, synthetic construct
  source 1..20: mol_type = other DNA
aagtcctcctcatgccccta

SEQ ID NO: 26 - DNA, 20 bp, synthetic construct
  source 1..20: mol_type = other DNA
ggtgtcccgtctaatggaga

SEQ ID NO: 27 - DNA, 20 bp, synthetic construct
  source 1..20: mol_type = other DNA
acacccaggattcacagagg

SEQ ID NO: 28 - DNA, 20 bp, synthetic construct
  source 1..20: mol_type = other DNA
gtgccaagggcctgaatgag

SEQ ID NO: 29 - DNA, 19 bp, synthetic construct
  source 1..19: mol_type = other DNA
gcaaaggctccaggtctga

SEQ ID NO: 30 - DNA, 21 bp, synthetic construct
  source 1..21: mol_type = other DNA
ccaacaccaacctgtccaagt

SEQ ID NO: 31 - DNA, 23 bp, synthetic construct
  source 1..23: mol_type = other DNA
agaggttattcctcgtcgtgcat

SEQ ID NO: 32 - DNA, 22 bp, synthetic construct
  source 1..22: mol_type = other DNA
agccgtgaccactgacaacgag

SEQ ID NO: 33 - DNA, 23 bp, synthetic construct
  source 1..23: mol_type = other DNA
gctgcatggttctgagtgctaag

(19) United States
(12) Patent Application Publication
(10) Pub. No.: US 2023/0151372 A1
(43) Pub. Date: May 18, 2023

(54) Composition and Method of Treatment for Heart Protection and Regeneration

(71) Applicant: Academia Sinica, Taipei (TW)

(72) Inventors: Patrick Ching-Ho Hsieh, Taipei (TW); Yuan-Yuan Cheng, Taipei (TW)

(73) Assignee: Academia Sinica, Taipei (TW)

(21) Appl. No.: 17/961,595

(22) Filed: Oct. 7, 2022

Related U.S. Application Data
(60) Provisional application No. 63/253,526, filed on Oct. 7, 2021.

(51) Int. Cl. C12N 15/52; C12N 15/86; A61P 9/10

(52) U.S. Cl. CPC C12N 15/52; C12N 15/86; A61P 9/10; C12N 2750/14143

(57) ABSTRACT

The present invention provides a gene delivery vehicle comprising a heterologous genome capable of upregulating the expression of HMGCS2 in human heart and, in particular, in the cardiomyocyte (CM). Upregulating the expression of HMBCS2 causes a metabolic switch that facilitates CM dedifferentiation and regeneration under myocardial infarction or hypoxic conditions. The present invention also provides a method of therapy for protection and/or regeneration of the human heart and, in particular, in the CM by administration of the composition of the present invention to the patient.

### CROSS REFERENCE TO RELATED APPLICATIONS

This application claims the benefit of U.S. Provisional Application No. 63/253,526, filed Oct. 7, 2021 which is herein incorporated in its entirety.

### REFERENCE TO AN ELECTRONIC SEQUENCE LISTING

The contents of the electronic sequence listing (Composition and Method of Treatment for Heart Protection and Regeneration.xml; Size: 57,880 bytes; and Date of Creation: Oct. 6, 2022) is herein incorporated by reference in its entirety.

### BACKGROUND OF THE INVENTION

Metabolic flexibility is essential for the heart to adapt to various changes in the microenvironment (Karwi et al., 2018), and changes in metabolism and substrate utilization are well-demonstrated in cardiomyocytes (CMs) during development and following injury. Proliferative fetal CMs favor glycolysis to generate ATP during cardiac development; however, soon after birth, CMs begin to utilize primarily aerobic fatty acid (FA) metabolism. During the same time period, neonatal human CMs rapidly lose their proliferative ability (Bergmann et al., 2015). As the heart enlarges through childhood, rod-shaped CMs undergo hypertrophy, rather than hyperplasia. When injured by hypoxic stress, CMs enlarge due to pathological hypertrophy and their sarcomeric structures become disorganized. During this process, they also regain a small amount of proliferative ability along with a metabolic switch to glycolysis (Neubauer., 2007). This suggests that CM metabolism, dedifferentiation, and proliferation are intrinsically linked. Yet, in adult mammals this adaptive response is not strong enough for complete or even adequate cardiac regeneration after injury. Therefore, there is a need to amplify the metabolic switch or reprogramming to induce substantially higher level of adult CM dedifferentiation and proliferation following injury to provide higher level of CM regeneration.

### SUMMARY OF THE INVENTION

The present invention provides a gene delivery composition comprising a gene delivery vehicle and a heterologous genome wherein the gene delivery vehicle houses or encapsulates the heterologous genome and wherein the heterologous genome comprises nucleic acid sequence at least 80%, 90% or 95% identical to SEQ. ID NO.:1. In an embodiment, the heterologous genome encodes human 3-hydroxy-3-methylglutaryl-CoA synthase 2 (mitochondrial) (HMGCS2) or its various isoforms. In an embodiment, the heterologous genome further comprises a 5′ primer site and a 3′ primer site flanking the nucleic acid sequence. In another embodiment, the heterologous genome encodes HMGCS2 enzyme or any of its functionally homologous forms. In an embodiment, the 5′ primer site comprises nucleotide sequence at least 80%, 90% or 95% identical to the nucleotide sequence of SEQ ID NO:2 and the 3′ primer site comprises nucleotide sequence at least 80%, 90% or 95% identical to the nucleotide sequence of SEQ ID NO:3. In another embodiment, the gene delivery vehicle comprises a nanoparticle. In an embodiment, the gene delivery vehicle comprises a recombinant adeno-associated virus (rAAV). In an embodiment, the rAAV comprises an AAV9 capsid.

The present invention also provides a method of treatment for cardiac ischemia comprising the step of providing a therapeutically effective amount of HMGCS2 to a patient. In an embodiment, the step of providing a therapeutically effective amount of HMGCS2 to the patient comprises the step of upregulating the expression of HMGCS2 in the patient's CM. In another embodiment, the step of upregulating the expression of HMGCS2 in the patient's CM comprises the step of administration of a therapeutically effective amount of the composition of claim 1 to the patient's heart. In an embodiment, step of administration of a therapeutically effective amount of the composition to the heart comprises administration of between about 10⁷-10¹⁸, about 10¹¹-10¹⁷ or about 10¹²-10¹³ of the rAAV particles. In an embodiment, the step of providing a therapeutic effective amount of HMGCS2 to the patient is performed before the cardiac ischemia. In another embodiment, the step of providing a therapeutic effective amount of HMGCS2 to the patient is performed after the cardiac ischemia. In an embodiment, the step of providing a therapeutic effective amount of HMGCS2 to the patient is performed 1 day, 2 days, 5, days, 10 days, 20 days or 30 after the cardiac ischemia.

The present invention also provides a method of treatment for cardiac ischemia comprising the step inducing a metabolic switch of adult cardiomyocyte (CM) using HMGCS2.

### BRIEF DESCRIPTION OF THE DRAWINGS

FIGS. 1A-1S show that in vivo CM-reprogramming induces metabolic switch, CM dedifferentiation and increased CM proliferation. FIG. 1A illustrates the experimental design for investigating adult CM reprogramming in vivo. FIG. 1B illustrates OSKM expression level and induction level in adult CMs after inducing OSKM reprogramming for 2 days. FIG. 1C depicts the flow cytometry analysis of isolated proliferative CMs through BrdU tracking in CM-reprogramming mice after OSKM induction. FIG. 1D depicts the percentage of proliferative CMs at each CM-reprogramming day determined by flow cytometry. FIG. 1E depicts the schematic diagram of intravital imaging protocol used for live investigating CM-reprogramming hearts after PBS or OSKM induction in vivo for 2 days. FIG. 1F depicts an investigation of CM alignment in the whole CM-reprogramming hearts by intravital microscopy after PBS or OSKM induction in vivo for 2 days. FIG. 1G depicts the morphology of CMs in CM-reprogramming hearts determined by length and width in intravital imaging data after PBS or OSKM induction in vivo for 2 days. Each dot represents one CM in one Ctrl or reprogramming heart. FIG. 1H depicts the aspect ratio determined by length-to-width ratio of each adult CMs of one control or CM-reprogramming mouse in intravital imaging data after PBS or OSKM induction in vivo for 2 days. FIG. 1I depicts the aspect ratio determined by length-to-width ratio of each CM-reprogramming mouse in intravital imaging data after PBS or OSKM induction specifically in CMs in vivo for 2 days. Each dot represents one mouse sample. FIG. 1J shows immunofluorescence staining of heart tissue sections showing morphology of proliferative CMs through H3P and WGA staining on CM-reprogramming hearts after PBS or OSKM induction for 2 days. Arrow heads represented H3P+ proliferative CMs. Scale bars were 50 μm. FIG. 1K shows the percentage of proliferative CM percentage (H3P+%) in the heart tissue sections of CM-reprogramming hearts after PBS or OSKM induction for 2 days. FIG. 1L depicts the morphology of H3P+ CMs in three CM-reprogramming hearts determined by length, width, and aspect ratio in heart tissue sections after OSKM induction in vivo for 2 days. Each dot represents one CM in one Ctrl or reprogramming heart. FIG. 1M shows immunofluorescence of heart tissue sections showing morphology of proliferative CMs through Aurora B Kinase (AURKB) and cardiac Troponin T (cTnT) staining on control or CM-reprogramming hearts after PBS or OSKM induction for 2 days. Arrow heads represented AURKB+/cTnT+ proliferative CMs. Scale bars were 25 μm. FIG. 1N shows the statistics of proliferative CM percentage (AURKB+%) in the heart tissue sections of CM-reprogramming hearts after PBS or OSKM induction for 2 days. FIG. 1O depicts the experimental design for discovering the detail mechanism for adult CM reprogramming at day 2 by microarray analysis. FIG. 1P depicts gene ontology analysis of gene expressional changes in adult CMs after PBS or OSKM induction for 2 days in vivo. FIG. 1Q is a heat map showing metabolism-related gene expressional changes in adult CMs after PBS or OSKM induction for 2 days in vivo. FIG. 1R and 1S show live imaging of CM-specific OSKM mice, related to FIG. 1A to 1Q. FIG. 1R shows OSKM RNA expression measured by real-time PCR in several tissues isolated from control or CM-reprogramming mice after doxycycline treatment for 2 days. FIG. 1S shows intravital live imaging of one construction in control or CM-reprogramming hearts after doxycycline treatment for 2 days.

FIGS. 2A to 2V show how cardiac-specific ketogenesis creates a systemic and specific metabolic switch along with mitochondrial changes, inducing CM dedifferentiation at CM-reprogramming day 2. FIG. 2A depicts the experimental design for metabolic profiling using LC-MS analysis. FIG. 2B shows hits detected by LC-MS analysis especially in both control and CM-reprogramming hearts. FIG. 2C depicts grouping of metabolic hits detected by LC-MS analysis in control or CM-reprogramming hearts. FIG. 2D shows the experimental design for metabolic profiling using a working heart system perfused with 13C-metabolites, detected by NMR. FIG. 2E depicts the oxidation percentage of control and CM-reprogramming hearts measured by 13C-glutamate level derived from different 13C-metabolic substrates through NMR analysis. FIG. 2F depicts ratio (CM-reprogramming to control hearts) of specific 13C-metabolites of control and CM-reprogramming hearts detected by NMR. FIG. 2G depicts the experimental design for measuring ketogenesis in the control or CM-reprogramming hearts. FIG. 2H depicts the HMG-CoA level detected by HPLC in the isolated mitochondria from control or CM-reprogramming hearts. FIG. 2I depicts the OHB level measured by OHB colorimetric assay in the isolated CMs from control or CM-reprogramming mice. FIG. 2J depicts the OCR detected by Seahorse analysis in the isolated CMs from control or CM-reprogramming mice. FIG. 2K shows the quantification of basal and maximal OCRs in the control or reprogramming CMs isolated from PBS or OSKM-treated hearts. FIG. 2L depicts the RNA expression of Hmgcs2 normalized by GAPDH in CMs isolated from control or OSKM-treated mice. FIG. 2M depicts protein expression of HMGCS2 in CMs isolated from control or OSKM-treated mice. FIG. 2N depicts a schematic diagram showing metabolic switch in adult CMs after OSKM induction for 2 days. FIG. 2O shows mitochondrial copy numbers detected by mtDNA through real-time PCR in control or reprogramming CMs isolated from PBS or OSKM-treated hearts. FIG. 2P shows mitochondrial RNA expression detected by real-time PCR in control or reprogramming CMs isolated from PBS or OSKM treated hearts. These RNA expressions were normalized by GAPDH. FIG. 2Q shows mitochondrial structure examined by TEM in isolated control or CM-reprogramming hearts. FIG. 2R shows mitochondrial size in isolated control or CM-reprogramming hearts, determined by TEM. FIG. 2S shows the aspect ratio of mitochondrial length-to-width in isolated control or CM-reprogramming, determined by TEM. FIG. 2T to 2V show cardiac function of control or CM-OSKM mice, related to FIG. 2A to 2S. FIG. 2T shows NMR peaks for measuring oxidation % of different metabolic substrates in control or CM-reprogramming hearts. FIG. 2U depicts cardiac function measured by echocardiography in control or CM-reprogramming hearts. FIG. 2V shows Western-blotting of phosphorylated DRP-1 on Ser616 or DRP-1 protein expression in control or CM-reprogramming CMs.

FIGS. 3A to 3S show that forced HMGCS2 overexpression increases adult CM dedifferentiation and proliferation for heart function improvement after myocardial infarction or under hypoxia when the forced HMGCS2 overexpression is effected before the myocardial infarction or imposition of the hypoxia environment. FIG. 3A depicts the experimental design for performing myocardial infarction (MI) in AAV9-EGFP or AAV9-HMGCS2 mice. FIG. 3B depicts heart function measured by echocardiography in AAV9-EGFP or AAV9-HMGCS2 mice. FIG. 3C depicts heart function measured by catheterization in AAV9-EGFP or AAV9-HMGCS2 mice. FIG. 3D depicts the fibrotic area in AAV9-EGFP or AAV9-HMGCS2 hearts shown by Masson Tri-chrome staining of heart tissue sections at post-MI day 21. FIG. 3E shows quantification of fibrotic percentage in AAV9-EGFP or AAV9-HMGCS2 hearts at post-MI day 21 measured by Masson Trichrome Staining. FIG. 3F shows immunofluorescence staining of heart tissue sections showing morphology of proliferative CMs through H3P and cTnT staining at the border zone of AAV9-EGFP or AAV9-HMGCS2 mice at post-MI day 3. Arrow heads represented H3P+/cTnT+ proliferative CMs. Scale bars were 50 μm. FIG. 3G shows quantification of proliferative CMs (H3P+%) in the heart tissue sections of at the border zone of AAV9-EGFP or AAV9-HMGCS2 mice at post-MI day 3. FIG. 3H shows immunofluorescence staining of heart tissue sections showing morphology of proliferative CMs through AURKB and cTnT staining at the border zone of AAV9-EGFP or AAV9-HMGCS2 mice at post-MI day 3. Arrow heads represented AURKB+/cTnT+ proliferative CMs. Scale bars were 25 μm. FIG. 3I shows quantification of proliferative CM percentage (AURKB+%) in the heart tissue sections at the border zone of AAV9-EGFP or AAV9-HMGCS2 mice at post-MI day 3. FIG. 3J shows experimental design for examining effects on forced HMGCS2 expression in hiPSC-CMs after Lenti-EGFP or Lenti-HMGCS2 infection. FIG. 3K shows protein expression of HMGCS2 measured by western-blot in Ctrl or HMGCS2 overexpressed hiPSC-CM under hypoxia. FIG. 3L shows OHB levels detected by OHB colorimetric assay in Ctrl or HMGCS2 overexpressed hiPSC-CM under hypoxia. FIG. 3M shows the morphology of control or HMGCS2 overexpressed hiPSC-CM under hypoxia. FIG. 3N shows the length of each control or HMGCS2 overexpressed hiPSC-CM under hypoxia. FIG. 3O shows the width of each control or HMGCS2 overexpressed hiPSC-CM under hypoxia. FIG. 3P shows the aspect ratio determined by length-to-width ratio of each control or HMGCS2 overexpressed hiPSC-CM under hypoxia. FIG. 3Q shows the proliferative ability determined by calculation of CM numbers of control or HMGCS2 overexpressed hiPSC-CM after culturing in hypoxia chamber for 24 hours. FIGS. 3R and 3S show Lentiviral infection efficiency in hiPSC-CMs, related to FIG. 3A to 3Q. FIG. 3R shows the morphology of BF in hiPSC-CMs. FIG. 3S shows the morphology of Lentiviral infection efficiency in hiPSC-CMs.

FIGS. 4A to 4I show that forced HMGCS2 overexpression increases adult CM dedifferentiation and proliferation for heart function improvement after myocardial infarction or under hypoxia when the forced HMGCS2 overexpression is effected after the myocardial infarction or imposition of the hypoxia environment. FIG. 4A depicts the experimental design for performing myocardial infarction (MI) in AAV9-EGFP or AAV9-HMGCS2 mice. FIG. 4B depicts heart function measured by echocardiography in AAV9-EGFP or AAV9-HMGCS2 mice. FIG. 4C depicts heart function measured by catheterization in AAV9-EGFP or AAV9-HMGCS2 mice. FIG. 4D shows the fibrotic area in AAV9-EGFP or AAV9-HMGCS2 mice hearts shown by Masson Tri-chrome staining of heart tissue sections at post-cI/R day 21. FIG. 4E shows quantification of infarct area % in heart sections of AAV9-EGFP or AAV9-HMGCS2 mice 21 day after cI/R. IS: infarct size; AAR: area at risk; LV: left ventricle. FIG. 4F depicts the fibrotic area in AAV9-EGFP or AAV9-HMGCS2 hearts shown by Masson Tri-chrome staining of heart tissue sections at post-MI day 21. FIG. 4G shows quantification of fibrotic percentage in AAV9-EGFP or AAV9-HMGCS2 hearts at post-MI day 21 measured by Masson Trichrome Staining. FIG. 4H shows immunofluorescence staining of heart tissue sections showing morphology of proliferative CMs through H3P and cTnT staining at the border zone of AAV9-EGFP or AAV9-HMGCS2 mice at post-MI day 3. Arrow heads represented H3P+/cTnT+ proliferative CMs. Scale bars were 50 μm. FIG. 4I shows quantification of proliferative CMs (H3P+%) in the heart tissue sections of at the border zone of AAV9-EGFP or AAV9-HMGCS2 mice at post-MI day 3.

### DETAILED DESCRIPTION OF THE INVENTION

The compositions of the present invention can comprise, consist of, or consist essentially of the essential elements and limitations of the invention described herein, as well as any of the additional or optional ingredients, components, or limitations described herein.

As used in the specification and claims, the singular form “a” “an” and “the” include plural references unless the context clearly dictates otherwise. For example, the term “a” cell includes a plurality of cells, including mixtures thereof.

“About” in the context of amount values refers to an average deviation of maximum ±20%, ±10% or ±5% based on the indicated value. For example, an amount of about 30 mg refers to 30 mg±6 mg, 30 mg±3 mg or 30 mg±1.5 mg.

A “therapeutically effective amount” is an amount sufficient to effect beneficial or desired results. A therapeutically effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets, By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, i.e., “sense” or “antisense” strands, can be packaged into any one AAV virion and both strands are equally infectious. The term “adeno-associated virus” (AAV) in the context of the present invention includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered.

By “recombinant virus” is meant a virus that has been genetically altered, e.g., by the deletion of endogenous nucleic acid and/or addition or insertion of a heterologous nucleic acid construct into the particle.

A “nucleic acid” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences. The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

A “coding sequence” or a sequence which “encodes” a particular protein, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the term “gene” or “recombinant gene” refers to a nucleic acid comprising an open reading frame encoding a polypeptide, including both exon and (optionally) intron sequences.

The term “heterologous” as it relates to nucleic acid sequences such as coding sequences and control sequences, denotes sequences that do not occur in nature or are not normally joined together in nature, and/or are not associated with a particular cell in nature. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “recombinant AAV virion,” or “rAAV virion” is defined herein as an infectious, replication-defective virus comprising an AAV protein shell encapsulating one or more heterologous nucleotide sequence that may be flanked on both sides by AAV ITRs. A rAAV virion may be produced in a suitable host cell comprising an AAV vector, AAV helper functions, and accessory functions. In this manner, the host cell may be rendered capable of encoding AAV polypeptides that are required for packaging the AAV vector containing a recombinant nucleotide sequence of interest into infectious recombinant virion particles for subsequent gene delivery.

“Homology” refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which allow for the formation of stable duplexes between homologous regions, followed by digestion with single stranded-specific nuclease(s), and size determination of the digested fragments. Two DNA, or two polypeptide sequences are “substantially homologous” to each other when at least about 80%, at least about 90% or at least about 95% of the nucleotides or amino acids match over a defined length of the molecules, as determined using the methods above.

A “functional homologue” or a “functional equivalent” of a given polypeptide may be molecules derived from the native polypeptide sequence, as well as recombinantly produced or chemically synthesized polypeptides that function in a manner similar to the reference molecule to achieve a desired result. Thus, a functional homologue of AAV Rep68 or Rep78 encompasses derivatives and analogues of those polypeptides, including any single or multiple amino acid additions, substitutions and/or deletions occurring internally or at the amino or carboxy termini thereof—so long as integration activity remains.

A “functional homologue” or a “functional equivalent” of a given adenoviral nucleotide region may be similar regions derived from a heterologous adenovirus serotype, nucleotide regions derived from another virus or from a cellular source, and recombinantly produced or chemically synthesized polynucleotides which function in a manner similar to the reference nucleotide region to achieve a desired result. Thus, a functional homologue of an adenoviral VA RNA gene region or an adenoviral E2A gene region encompasses derivatives and analogues of such gene regions-including any single or multiple nucleotide base additions, substitutions and/or deletions occurring within the regions, so long as the homologue retains the ability to provide its inherent accessory function to support AAV virion production at levels detectable above background.

A “gene delivery vehicle” comprises any method or composition capable of fully or partially encapsulating or housing genome to be carried or delivered to a desired target in a human body such as a cardiomyocyte. The gene delivery vehicle may be biological, chemical or physical in nature or a combination thereof and provides protection for the genome while being carried to be delivered to the desired target. Biological gene delivery vehicle may be bacterial or viral such as rAAV. Chemical gene delivery vehicle may be polymeric particles, liposomes, polymer-lipid hybrid nanoparticles, other biocompatible materials, or combinations thereof. Physical gene delivery vehicle may comprise microinjection, electroporation, ultrasound, gene dun, hydrodynamic applications, or combinations thereof.

The present invention provides a cardiac protection and/or regeneration composition and method of treatment based on 3-hydroxy-3-methylglutaryl-CoA synthase 2 (mitochondrial) (HMGCS2).

HMGCS2 is an enzyme in humans that is encoded by the HMGCS2 gene. A complete human HMGCS2 sequence hereby defined as SEQ ID NO. 1 is listed in the sequence listing section below as well as in Rojnueangnit et al. Eur J Med Genet. 2020 December; 63(12):104086 which is hereby incorporated in its entirety. The HMGCS2, belonging to the HMG-CoA synthase family, is known to be a mitochondrial enzyme that catalyzes the second and rate-limiting reaction of ketogenesis, a metabolic pathway that provides lipid-derived energy for various organs during times of carbohydrate deprivation, such as fasting, by addition of a third acetyl group to acetoacetyl-CoA, producing HMG-CoA. Mutations in this gene are associated with HMG-CoA synthase deficiency. Alternatively spliced transcript variants encoding different isoforms have been found for this gene such as those published by Puisac et al., Mol Biol Rep. 2012. 39:4777-4785 which is hereby incorporated in its entirety.

Cardiac regeneration after injury in adult mammals including adult humans is limited by the low proliferative capacity of cardiomyocytes (CMs). However, certain animals such as zebrafish, newts, and neonatal mice readily regenerate lost myocardium via a process involving dedifferentiation, which unlocks their proliferative capacities. Inspired by this concept, in Example 1 detailed below, we created an experimental model comprising mice with inducible, CM-specific expression of the Yamanaka factors, enabling adult CM reprogramming in vivo. Specifically, two days following induction by doxycycline, adult CMs presented a dedifferentiated phenotype and increased proliferation of CM in vivo indicating cardiac regeneration. Moreover, in Example 2 detailed below, microarray analysis revealed that metabolic changes were central to this process. In particular, metabolic switch from fatty acid to ketone utilization as indicated by increase in ketogenic enzyme HMGCS2.

Furthermore, Examples 3 and 4 showed that HMGCS2 overexpression by exogenous means is capable of rescuing cardiac function following ischemic injury when HMGCS2 overexpression is effect before (Example 3) as well as after (Example 4) the ischemic injury. Thus, experiments disclosed in the Examples below reveal that HMGCS2-induced ketogenesis leads to metabolic switch in adult CMs during early reprogramming, and this metabolic adaptation substantially increases adult CM dedifferentiation, facilitating cardiac regeneration after injury.

Therefore, embodiments of the present invention encompass various compositions capable of providing a therapeutically effective amount of HMGCS2, variants thereof disclosed herein or functional homologues to a patient capable of effecting cardiac protection and/or regeneration in infarcted or injured areas of the heart of the patient. The composition of the present invention may also encompass various compositions which when administered to the patient effects expression of a therapeutically effective amount of HMGCS2, variants thereof disclosed herein or functional homologues in cells of the patient such as cardiomyocyte capable of effecting cardiac protection and/or regeneration in infarcted or injured areas of the heart, including but not limited to compositions capable of effecting viral-mediated gene delivery, naked DNA delivery, mRNA delivery, transfection methods etc. . . . The composition of the present invention may also encompass various compositions which when administered to the patient effects expression of a therapeutically effective amount of HMGCS2, variants thereof disclosed herein or functional homologues in cells of the patient capable of effecting cardiac protection and/or regeneration in infarcted or injured areas of the heart, including but not limited to compositions comprising gene delivery vehicles housing or fully or partially encapsulating the HMGCS2 genome capable of effecting viral-mediated gene delivery, naked DNA delivery, mRNA delivery, transfection methods etc . . . .

In an embodiment, the composition of the present invention comprises rAAV comprising heterologous nucleic acids encoding HMGCS2, variants thereof disclosed herein or functional homologues capable of effecting cells of the patient to express HMGCS2, variants thereof disclosed herein or functional homologues at a substantially higher level than without the rAAV. AAV is a parvovirus belonging to the genus Dependovirus. Although it can infect human cells, AAV has not been associated with any human or animal disease and is stable at a wide range of physical and chemical conditions. In addition, making AAV a desirable gene delivery vehicle.

The wild type AAV genome is a linear, single-stranded DNA molecule containing 4681 nucleotides. It comprises an internal non-repeating genome flanked on each end by inverted terminal repeats (ITRs) which are approximately 145 base pairs (bp) in length. The ITRs have multiple functions, including originals of DNA replication and as packaging signals for the viral genome.

The internal non-repeated portion of the wild type AAV genome includes two large open reading frames, known as the AAV replication (rep) and capsid (cap) genes. The rep and cap genes code for viral proteins that allow the virus to replicate and package the viral genome into a virion. In particular, a family of at least four viral proteins an expressed from the AAV rep region, Rep 78, Rep 69, Rep 52 and Rep 40, named according to their apparent molecular weight, the AAV cap region encodes at least three proteins, VP1, VP2 and VP3.

AAV can be engineered to deliver genes of interest as rAAV by deleting at least some of the internal non-repeating portion of the AAV genome such as rep and cap and inserting one or more heterologous gene between the ITRs. In an embodiment, the rAAV of the present invention comprises AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered or a combination thereof.

The heterologous gene may be functionally linked to a heterologous promoter (constitutive, cell-specific, or inducible) capable of driving gene expression in the patient's target cells under appropriate conditions. Termination signals, such as polyadenylation sites, can also be included.

Therefore, in an embodiment, the composition of the present invention comprises rAAV with genome encoding HMGCS2, variants thereof disclosed herein or functional homologues such that a patient's cells infected with rAAV express HMGCS2, variants thereof disclosed herein or functional homologues as disclosed or shown in the Examples. In another embodiment, the composition of the present invention comprises AAV9 with genome encoding HMGCS2, variants thereof disclosed herein or functional homologues disclosed herein such that a patient's cells infected with rAAV express HMGCS2, variants thereof disclosed herein or functional homologues in the heart tissue as shown in the Examples. In an embodiment, the genome encoding HMGCS2, variants thereof disclosed herein or functional homologues comprises primers. Such primer may comprise.

Primer-F→
(SEQ ID NO. 2)
ATACATGGCCAAAAGATGTGGGC
Primer-R→
(SEQ ID NO. 3)
GCACGACGGGACACCGGGCATAC

In an embodiment, the rAAV genome comprises nucleotide sequences described above flanked by ITRs. In another embodiment, the nucleotide sequence encoding HMGCS2, variants thereof disclosed herein or functional homologues is functionally linked to a heterologous promoter capable of driving gene expression in the patient's target cells such as cardiomyocytes. Such promoters can include constitutive, cell-specific or inducible promoters. In an embodiment, the composition of the present invention further comprises αMHC promoter to induce HMGCS2 expression to target cardiomyocyte. In an embodiment the αMHC promoter comprises entire intergenic region between the β-MHC gene (upstream) and the αMHC gene with sequence as detailed in Subramaniam et al. J Biol Chem. 1991 Dec. 25; 266(36):24613-20 which is hereby incorporated in its entirety.

In an embodiment, the genome of the rAAV composition of the present invention is lacking one or more rep and cap genes, rendering the rAAV of the present invention unable to reproduce in a patient. The rAAV composition of the present invention may comprise the capsid of any known AAV serotypes such as AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered or a combination thereof. In another embodiment, since AAV-9 is known to specifically target the heart, in an embodiment, the composition of the present invention comprises rAAV-9 capsid comprising nucleotide sequence encoding HMGCS2, variants thereof disclosed herein or functional homologues.

In an embodiment, the composition of the present invention comprises genome fully or partially encapsulated in lipid formulation wherein the genome encodes HMGCS2 or any variants thereof as disclosed and lipid formulation comprises liposomes or polymeric nanoparticles. In another embodiment, the composition of the present invention comprises mRNA housed or encapsulated in lipid formulation wherein the mRNA encodes HMGCS2 or any variants thereof as disclosed and lipid formulation comprises liposomes or polymeric nanoparticles. Methods of preparation of these compositions are disclosed in U.S. Pat. No. 10,086,143 which is hereby incorporated in its entirety.

The present invention also provides a method of treatment for cardiac ischemia or heart diseases involving metabolic changes or loss of or injury to cardiomyocytes comprising the step of administering a therapeutically effective amount of any of the disclosed compositions of the present invention to a patient in need. In an embodiment, the present invention comprises a method of treatment for cardiac ischemia or heart diseases involving metabolic changes or loss of or injury to cardiomyocytes comprising the step of parenteral administration of a therapeutically effective amount of rAAV comprising nucleic acid encoding HMGCS2. In an embodiment, the dose range comprises between about 10⁷-10¹⁸, about 10¹¹-10¹⁷ or about 10¹²-10¹³ of the rAAV particles comprising nucleic acid encoding HMGCS2. In another embodiment, the present invention comprises a method of treatment for cardiac ischemia or heart diseases involving metabolic changes or loss of cardiomyocytes comprising the step of administration of a therapeutically effective amount of rAAV comprising nucleic acid encoding HMGCS2, variants thereof disclosed herein or functional homologues parenterally at and near the border region of the ischemia. In an embodiment, a method of treatment for cardiac ischemia or heart diseases involving metabolic changes or loss of cardiomyocytes comprising the step of administration of rAAV comprising nucleic acid encoding HMGCS2, variants thereof disclosed herein or functional homologues by perfusion of the heart.

In an embodiment, the method of the present invention comprises administration of HMGCS2 enzyme to the patient. In an embodiment, the method of the present invention comprises administration of HMGCS2 enzyme to the heart of the patient. In an embodiment, the method of the present invention comprises administration of HMGCS2 enzyme to the CM injured area of the patient. In an embodiment, the method of the present invention comprises administration of HMGCS2 enzyme to the border region of the CM injured area of the patient.

In all of the embodiments of the method of the present invention disclosed herein, the administration time may be prior to the cardiac ischemia. Alternatively, in all of the embodiments of the method of the present invention disclosed herein, the administration time may be after cardiac ischemia such as about 1 hour to about one month after the injury such as about 1 hour, about 3 hours, about 10 hours about 24 hours, about 2 days, about 4 days, about 10 days about 15 days about 20 days, about 25 days or about 30 days including any numbers and number ranges falling within these values. In all of the embodiments of the method of the present invention disclosed herein, the administration method may comprise parenteral administration to the patient and, in some embodiment, to the heart of the patient.

It is to be understood that both the foregoing general description and the following detailed description are exemplary and explanatory only and are not restrictive of the invention, as claimed.

These and other changes can be made to the technology in light of the detailed description. In general, the terms used in the following disclosure should not be construed to limit the technology to the specific embodiments disclosed in the specification, unless the above detailed description explicitly defines such terms. Accordingly, the actual scope of the technology encompasses the disclosed embodiments and all equivalent ways of practicing or implementing the technology.

It can be appreciated by those skilled in the art that changes could be made to the examples described without departing from the broad inventive concept thereof. It is understood, therefore, that this invention is not limited to the particular examples disclosed, but it is intended to cover modifications within the spirit and scope of the present invention as defined by the appended claims.

### Examples

Experimental Methods and Materials

Material and Methods

Animals

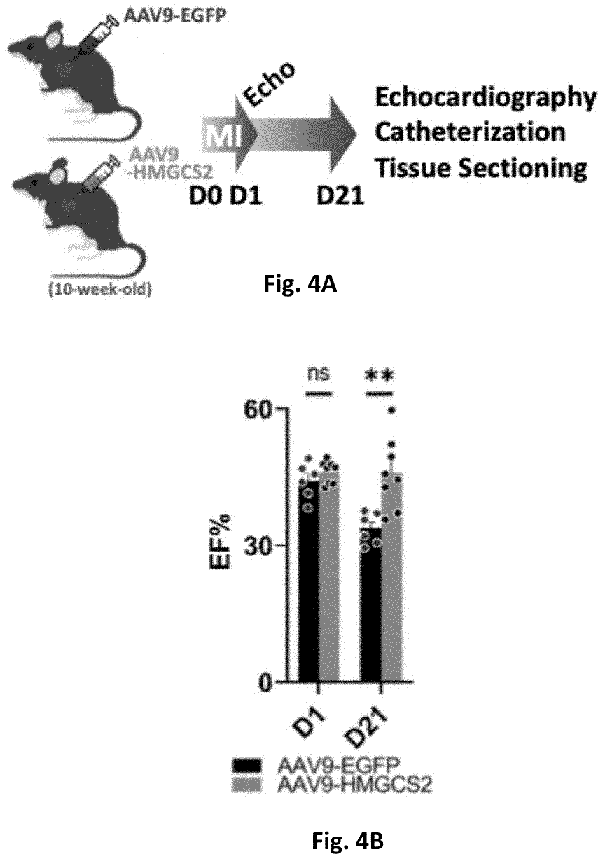

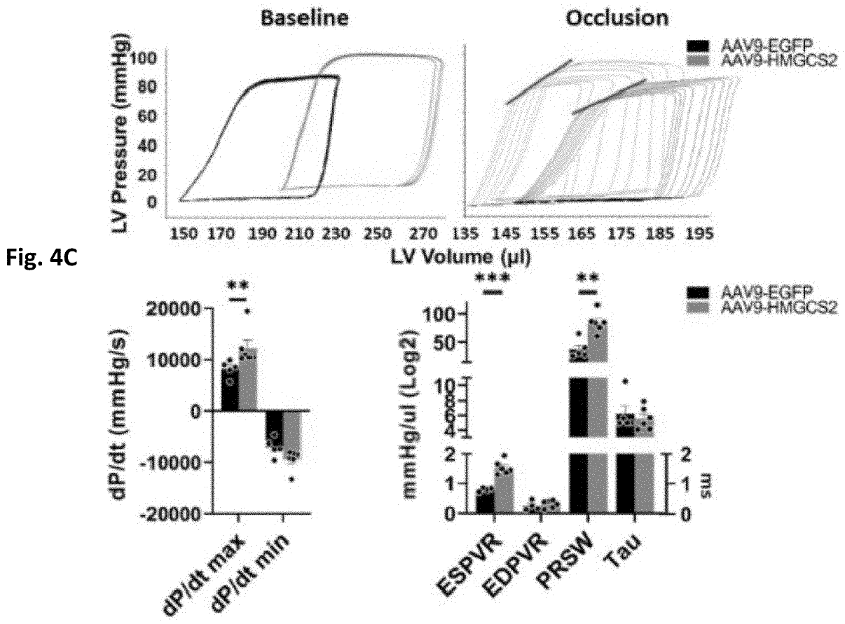

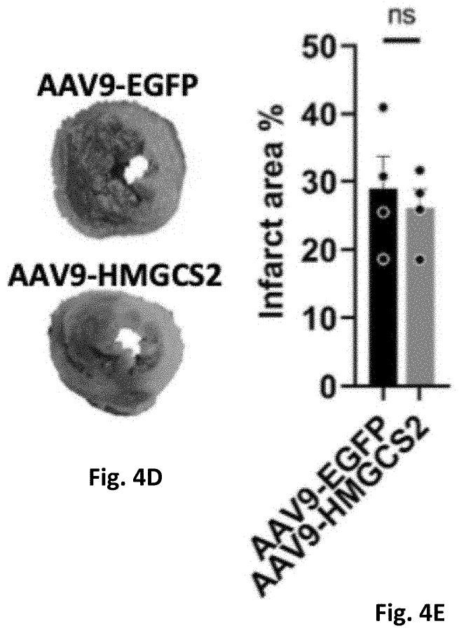

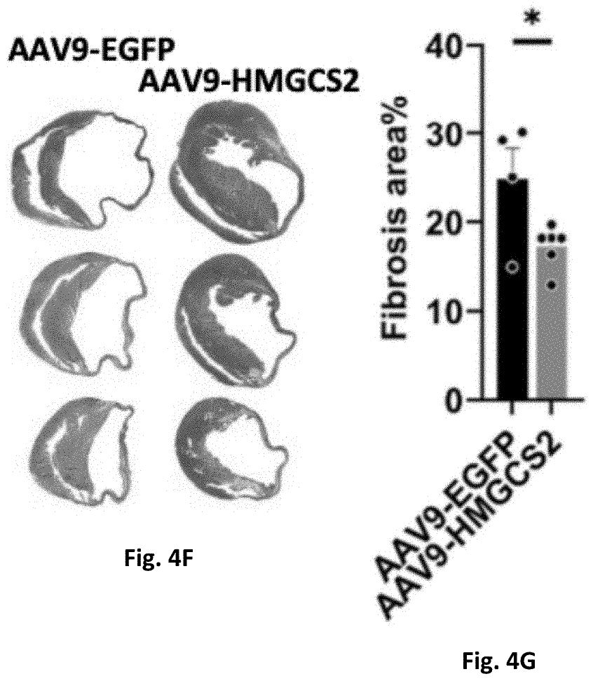

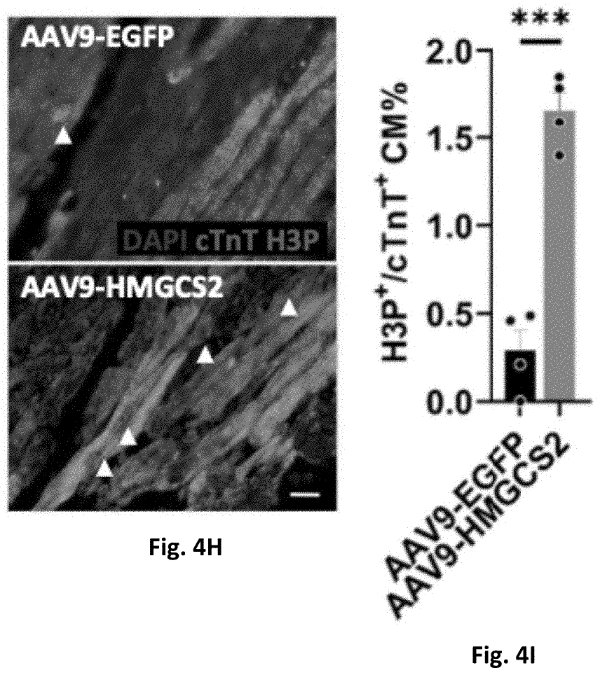

All animal experiments were conducted in accordance with the Guides for the Use and Care of Laboratory Animals (ARRIVE guidelines), and all of the animal protocols have been approved by the Experimental Animal Committee, Academia Sinica, Taiwan. Myh6-rtTA mice (Stock No: Jam8585) was purchased from MMRRC. Collal-tetO-OSKM mice (Stock No: 011001) and Myh6-CRE (Stock No: 011038) were both purchased from Jackson lab. Conditional HMGCS2 knockout mice were generated by inserting 2 1oxP fragments into the regions before and after exon 2 (FIG. 4A) through CRISPR/Cas9 technique. All mice were housed in individually ventilated cages (IVCs) system in animal core facility at Academia Sinica. Doxycycline treatment (Sigma-Aldrich, D9891) was administrated by intraperitoneal injection at 2 mg per 25 g mouse (Stadtfeld et al., 2010).

Adult CM Isolation

Adult ventricular CMs were isolated from mice on a Langendorff apparatus. After heparinization for 10 mins, the heart was removed from the anaesthetized mice and then was cannulated for retrograde perfusion with Ca2+-free Tyrode solution (NaCl 120.4 mmol/l, KCl 14.7 mmol/l, KH2PO4 0.6 mmol/l, Na2HPO4 0.6 mmol/l, MgSO4 1.2 mmol/l, HEPES 1.2 mmol/l, NaHCO3 4.6 mmol/l, taurine 30 mmol/l, BDM 10 mmol/l, glucose 5.5 mmol/1). After perfusion, the enzyme solution containing Ca2+-free Tyrode solution supplemented with collagenase B (0.4 mg/g body weight, Roche), collagenase D (0.3 mg/g body weight, Roche), and protease type XIV (0.05 mg/g body weight, Sigma-Aldrich) was perfused to digest the hearts for 10 mins. The ventricle was then cut from the cannula and teased into small pieces in the enzyme solution and then neutralized by the Ca2+-free Tyrode solution containing 10% FBS. Adult CMs were dissociated from the digested tissues by gentle pipetting and collected after removing the debris by filtering through a nylon mesh with 100 μm pores.

RNA Isolation and Real-Time PCR

Total RNA was isolated from frozen LV tissue or from isolated CMs using Trizol buffer (Invitrogen), and cDNA was synthesized using SuperScript IV reverse transcriptase and random hexamers according to manufacturers' guidelines. Real-time PCR was performed using SYBR green (Bio-Rad), and the primers are described in the Table Si. The mRNA levels in each sample were normalized to GAPDH RNA levels.

Flow Cytometry

Cells were fixed with 4% paraformaldehyde and permeabilized with 90% methanol on ice. The single cell suspension was further stained with anti-BrdU antibody (ab8152) for 30 mins then washed with PBS. After incubating with secondary antibody conjugated with Alexa fluor-488 or Alexa fluor-568 (Life Technologies) for another 30 mins, samples suspended in PBS were measured by LSRII SORP (Becton Dickinson) and analyzed by FlowJo Software (Treestar, Ashland, Oreg.).

Intravital Imaging

The multiphoton intravital imaging system was performed following the procedure published in previous study (Vinegoni., 2015). In brief, mice were anesthetized by 1.5% isoflurane (Minrad) and membrane potential dye (Di-2-ANEPEQ) was injected intravenously to examine live imaging of heart tissue was performed using a multi-photon scanning microscope.

Immunofluorescence

The tissue sections were deparaffinized, rehydrated, and antigens retrieved by boiling twice in sodium citrate solution. The sections were incubated with blocking buffer (5% goat serum and FBS) for 1 hour, and then stained with primary antibody including histone H3 phosphorylated at serine 10 (Millipore), and anti-cardiac troponin T (DSHB) at 4° C. overnight. Samples were incubated in secondary antibodies conjugated with Alexa fluor-488 or Alexa fluor-568 (Life Technology) for 1 h at room temperature. After PBS washing, the nuclei were stained with DAPI (Life Technologies) for 5 min.

Transcriptomic Analysis

Samples from control or reprogramming CMs were hybridized to a Mouse Oligo Microarray (Agilent) following the manufacturer's procedure, and arrays were scanned with Microarray Scanner System (Agilent). All CEL files were analyzed by GeneSpring GX software (Agilent) with quantile normalization and median polish probe summarization using the control group as a baseline. The expression levels in the first quantile were filtered out to remove noise. Genes were defined as differentially expressed if they had fold changes of at least ±2 combined with the Student's t-test (P<0.05) with the Benjamini-Hochberg adjustment for false discovery rate (FDR). Gene Ontology analysis was conducted using DAVID software (Huang et al, 2009). The biological replicates were two for control or reprogramming CM isolated from doxycycline treated CM-OSKM mice.

LC-MS Untargeted Profiling

Hearts were isolated from control or reprogramming mice at reprogramming day 2. After removing the atria and aorta, samples were frozen in liquid nitrogen and then prepared for LC-MS metabolic profiling. The whole profiling experiments including sample preparation followed a previously published procedure (Wang et al., 2015).

13C NMR Spectroscopy and Analysis

Mouse hearts were isolated and perfused with unlabeled mixed-substrate buffer (in mM; NaCl 118 mM, NaHCO3 25 mM, KCl 4.1 mM, CaCl2) 2 mM, MgSO4 1.2 mM, KH2PO4 1.2 mM, EDTA 0.5 mM, glucose 5.5 mM, mixed long-chain fatty acids bound to 1% albumin 1 mM, lactate 1 mM, and insulin 50 μU/mL) for 20 minutes and 13C-labeled mixed-substrate buffer for another 40 minutes. 13C-labeled mixed-substrate buffer was divided into 2 groups; one contained [U-13C]glucose and [1,4-13C] OHB and unlabeled mixed FA and Lactate, the other group contained [U-13C] mixed FA and [1,4-13C] Lactate and unlabeled glucose and OHB. After perfusion, the hearts were frozen in liquid nitrogen, homogenized and extracted in perchloric acid, and then neutralized by KOH. The hearts were then lyophilized and dissolved in deuterium oxide (D20) supplemented with internal standard Sodium trimethylsilyl propionate. A Bruker Avance III 600 MHz NMR Spectrometer was used to present proton-decoupled 13C NMR spectra of each heart sample, and spectra were generated by Fourier transformation following multiplication of the free-induction decays (FIDs) by an exponential function. The peak areas of each 13C-metabolites were analyzed using Bruker TopSpin 4.0.2.

High Performance Liquid Chromatography

An HPLC system Dionex Ultimate 3000 (ThermoFisher Scientific, Waltham, Mass., USA), with a Varian 380-LC (Varian, Palo Alto, Calif., USA) evaporative light-scattering detector was employed. The conditions used followed a published procedure (Heijden et al., 1994). In brief, the condition was used as follows: Column: Hypersil ODS (AMT, Wilmington, Del., USA), 250×4.6 mm, particle diameter 5 μm without precolumn. Solvent system: 0.2 M sodium phosphate buffer, pH 5.0, containing 4.5% (v/v) acetonitrile; flow rate: 1.5 ml/min. The compounds were detected by UV at 254 nm.

Transmission Electron Microscopy

To monitor mitochondria ultrastructure, transmission electron microscopy was used as described previously (Karamanlidis et al., 2013). Briefly, freshly collected samples from the apex of the mouse hearts were dissected in 1 mm³ sections and immediately fixed with 2% glutaraldehyde in 0.1 M phosphate buffered saline, and then fixed with 1% osmium tetroxide. After the samples were dehydrated in ethanol and embedded in epon resin, ultrathin sections were prepared and counterstained with uranyl acetate and lead citrate. The stained sections were examined under a Transmission Electron Microscope (JEOL1230). Mitochondrial number was counted in total of 10 images per heart (45 m2 at ×12000 magnification, n=3 hearts per group). Data were expressed as fold changes relative to WT.

Mitochondria Isolation

Mitochondria were collected from isolated hearts by sequential centrifugation (Boehm et al., 2001). In brief, hearts were isolated and rinsed with mitochondrial isolation buffer (250 mM Sucrose, 10 mM Tris-HCL, and 3 mM EDTA, pH 7.4). Heart tissue was minced in mitochondrial isolation buffer and was homogenized by a homogenizer with Teflon pestle. The homogenate was centrifuged at 800 g for 10 min at 4° C. to remove tissue debris. The supernatant was further centrifuged at 8000 g for 15 min at 4° C. to collect mitochondria.

Myocardial Ischemia and Reperfusion

C57BL/6 mice (10 weeks old) were randomized and anesthetized by isofluorane inhalation, endotracheally intubated, and placed onto a rodent ventilator. The left anterior descending (LAD) coronary artery was visualized and occluded with a prolene suture for 45 mins after first removing the pericardium. After confirming the whitening region of the left ventricle, the occluded LAD was released. EF % between 55-60% one day after occlusion was considered a successful cI/R model.

Determination of Infarct Size

Infarct and remote area performed by Myocardial I/R was determined by Evans blue/TTC double staining as described previously (Bohl et al., 2009). In brief, the ligature around the LAD was re-tied after 24 hours of reperfusion. Injection of 1 ml 1% Evans blue dye through heart apex and the heart was excised and then frozen in −20° C. refrigerator for 15 minutes and sliced into four 1 mm-thick slices. The slides were stained with 1% triphenyltetrazolium chloride (TTC, Sigma) in PBS at 37° C. for 10 minutes and photographed. The area at risk (AAR) was identified as red (TTC-stained) and white (infarct) areas. AAR, IR, and total LV area were measured by Image J software (NIH).

Western Blot Analysis and Immunoprecipitation

Myocardial tissues were frozen and lysed in RIPA buffer with a protease inhibitor cocktail. Protein samples (20 μg) were separated by SDS-PAGE and transferred to a PVDF membrane. The membrane was blocked in 5% skimmed milk and probed with primary antibodies overnight at 4° C.: HMGCS2 (sc-393256) and GAPDH (MAB374), followed by corresponding secondary antibodies. The membrane then was developed with ECL and the signal intensities were visualized by a Supersignal chemiluminescence detection kit (Pierce) and analyzed with Image J software (NIH).

Adeno-Associated Virus Production

AAV9 was produced by triple-transfection procedures using CMV-HMGCS2/CMV-EGFP plasmid, with a plasmid encoding Rep2Cap9 sequence and an adenoviral helper plasmid pHelper in 293 cells. Virus was purified by two cesium chloride density gradient purification steps through ultracentrifugation followed by dialysis against 5 rounds of PBS buffer change. Viral titers were determined by qPCR.

The primers to amplify full gene sequence of HMGCS2 were listed below.

Primer-F→
(SEQ ID NO. 2)
ATACATGGCCAAAAGATGTGGGC
Primer-R→
(SEQ ID NO. 3)
GCACGACGGGACACCGGGCATAC

Lentivirus Production

293 cells were seeded in 10-cm-diameter dishes 24 h prior to transfection using PolyJet (SL10068). The PLKO3.1-EGFP or PLKO3.1-HMGCS2 vector plasmids was each cotransfected together with psPAX2 and pMD2.G in a ratio of 5:4:1 (total 9 ag). After 12-18 hours of transfection, the culture medium (DMEM-HG) was changed and the viral supernatant was collected after 48 and 72 hours of transfection.

Primers Used in various RNA isolation and Real-Time PCR are listed in Table 1 below

TABLE 1

| Name | Sequence (5′ to 3′) |
|---|---|
| GAPDH-F | (SEQ ID NO. 4) |
| | CAT CAC TGC CAC CCA GAA GAC TG |
| GAPDH-R | (SEQ ID NO. 5) |
| | ATG CCA GTG AGC TTC CCG TTC AG |
| mOct4-F | (SEQ ID NO. 6) |
| | CCT GCA GAA GGA GCT AGA ACA GT |
| mOct4-R | (SEQ ID NO. 7) |
| | TGT TCT TAA GGC TGA GCT GCA A |
| mSox2-F | (SEQ ID NO. 8) |
| | GCA CAT GAA CGG CTG GAG CAA CG |
| mSox2-R | (SEQ ID NO. 9) |
| | TGC TGC GAG TAG GAC ATG CTG TAG G |
| mKlf4-F | (SEQ ID NO. 10) |
| | GAA ATT CGC CCG CTC CGA TGA |
| mKlf4-R | (SEQ ID NO. 11) |
| | CTG TGT GTT TGC GGT AGT GCC |
| cMyc-F | (SEQ ID NO. 12) |
| | GCC CCC AAG GTA GTG ATC CT |
| cMyc-R | (SEQ ID NO. 13) |
| | GTC CTC GTC TGC TTG AAT GG |
| mtDNA-F | (SEQ ID NO. 14) |
| | CGA AAG GAC AAG AGA AAT AAG G |
| mtDNA-R | (SEQ ID NO. 15) |
| | CTG TAA AGT TTT AAG TTT TAT GCG |
| mtCox1-F | (SEQ ID NO. 16) |
| | AGT CTA CCC ACC TCT AGC CG |
| mtCox1-R | (SEQ ID NO. 17) |
| | TGT GTT ATG GCT GGG GGT TT |
| mtAtp6-F | (SEQ ID NO. 18) |
| | TCC ACA CAC CAA AAG GAC GAA |
| mtAtp6-R | (SEQ ID NO. 19) |
| | CCA GCT CAT AGT GGA ATG GCT |
| mtAtp8-F | (SEQ ID NO. 20) |
| | CAT CAC AAA CAT TCC CAC TGG C |
| mtAtp8-R | (SEQ ID NO. 21) |
| | TGA GGC AAA TAG ATT TTC GTT CAT T |
| mtCox2-F | (SEQ ID NO. 22) |
| | GAC GAA ATC AAC AAC CCC GT |
| mtCox2-R | (SEQ ID NO. 23) |
| | TAG CAG TCG TAG TTC ACC AGG |
| mtNd2-F | (SEQ ID NO. 24) |
| | CAA GGGATC CCA CTG CAC AT |
| mtNd2-R | (SEQ ID NO. 25) |
| | AAG TCC TCC TCA TGC CCC TA |
| Hmgcs2-F | (SEQ ID NO. 26) |
| | GGT GTC CCG TCT AAT GGA GA |
| Hmgcs2-R | (SEQ ID NO. 27) |
| | ACA CCC AGG ATT CAC AGA GG |
| βMhc-F | (SEQ ID NO. 28) |
| | GTG CCA AGG GCC TGA ATG AG |
| βMhc-R | (SEQ ID NO. 29) |
| | GCA AAG GCT CCA GGT CTG A |
| αMhc-F | (SEQ ID NO. 30) |
| | CCA ACA CCA ACC TGT CCA AGT |
| αMhc-R | (SEQ ID NO. 31) |
| | AGA GGT TAT TCC TCG TCG TGC AT |
| Pgc1α-F | (SEQ ID NO. 32) |
| | AGC CGT GAC CAC TGA CAA CGA G |
| Pgc1α-R | (SEQ ID NO. 33) |
| | GCT GCA TGG TTC TGA GTG CTA AG |

### Example 1—In Vivo CM-Reprogramming Induces Metabolic Switch, CM Dedifferentiation and In-Creased CM Proliferation

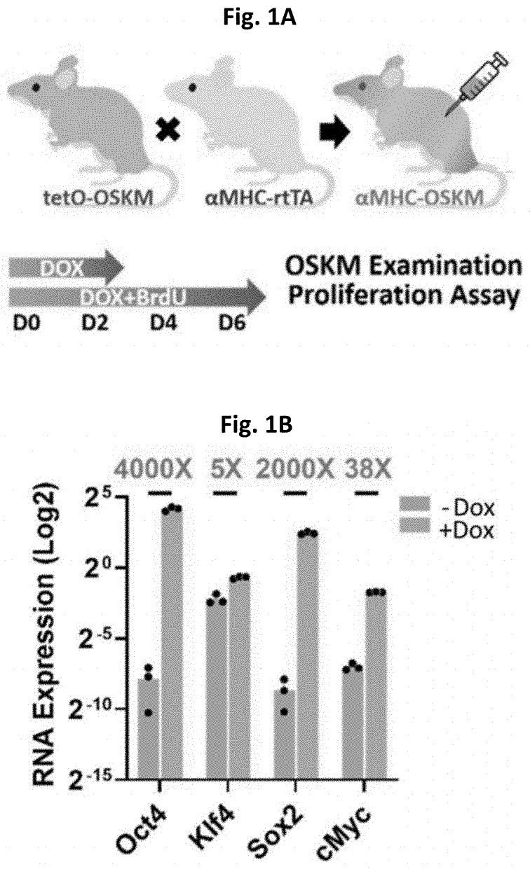

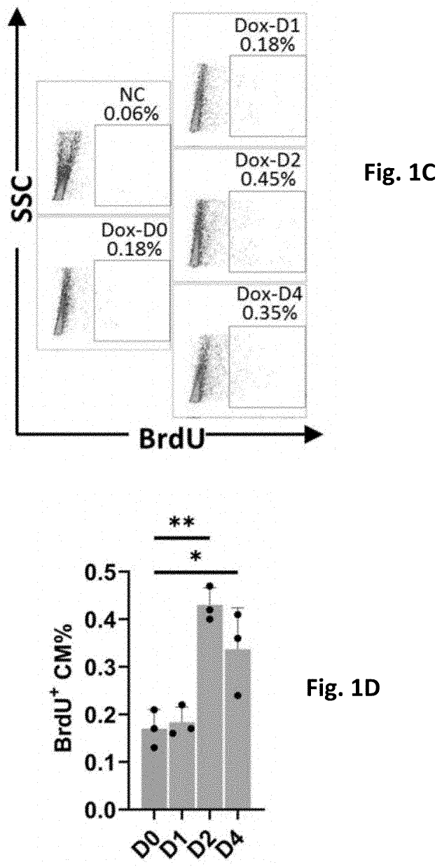

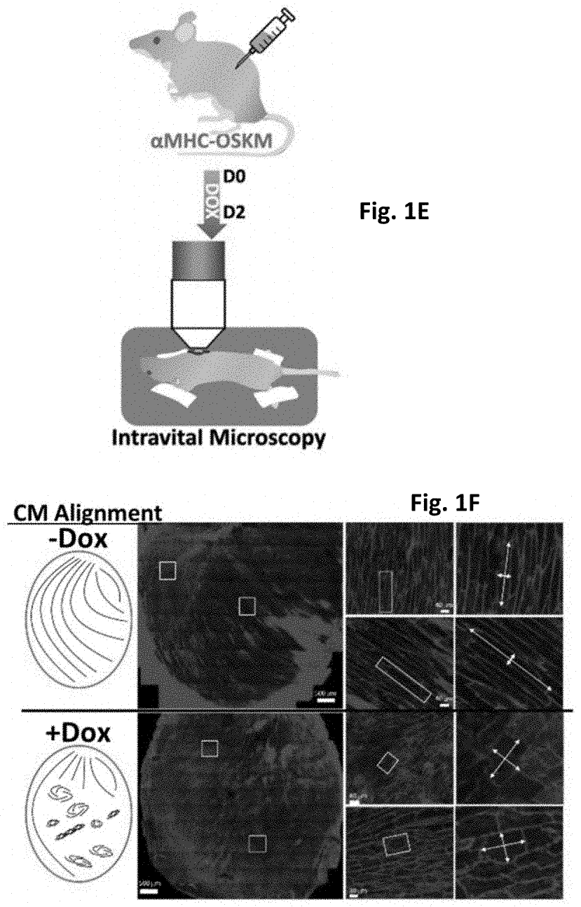

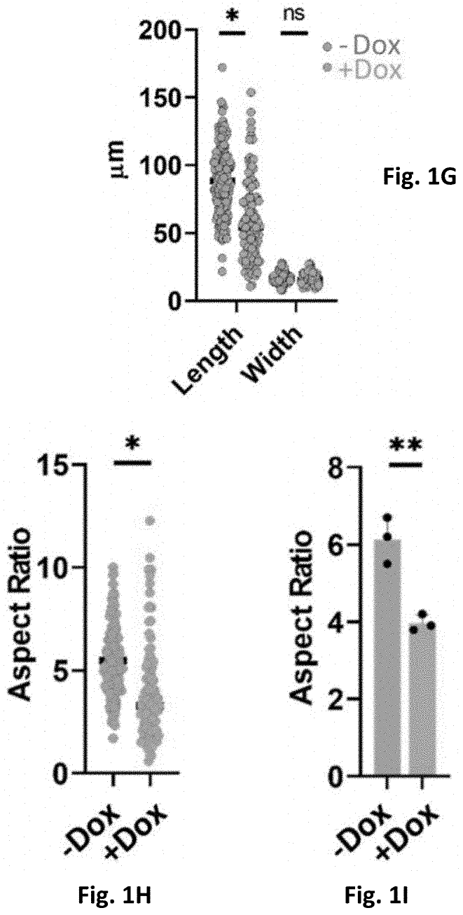

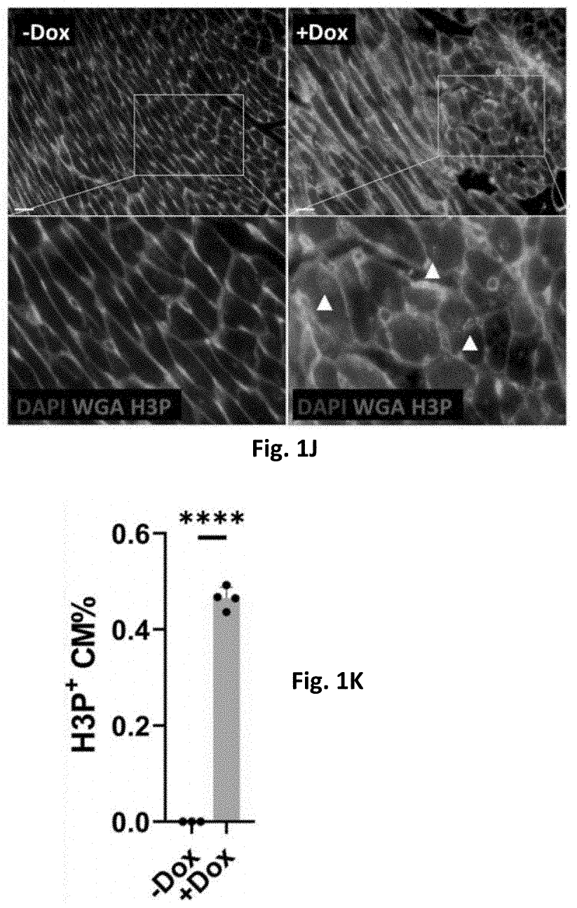

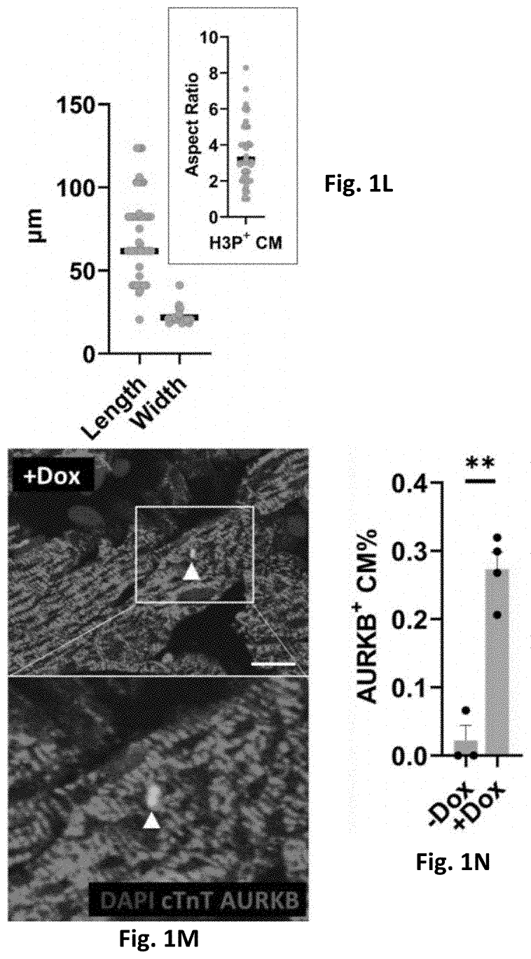

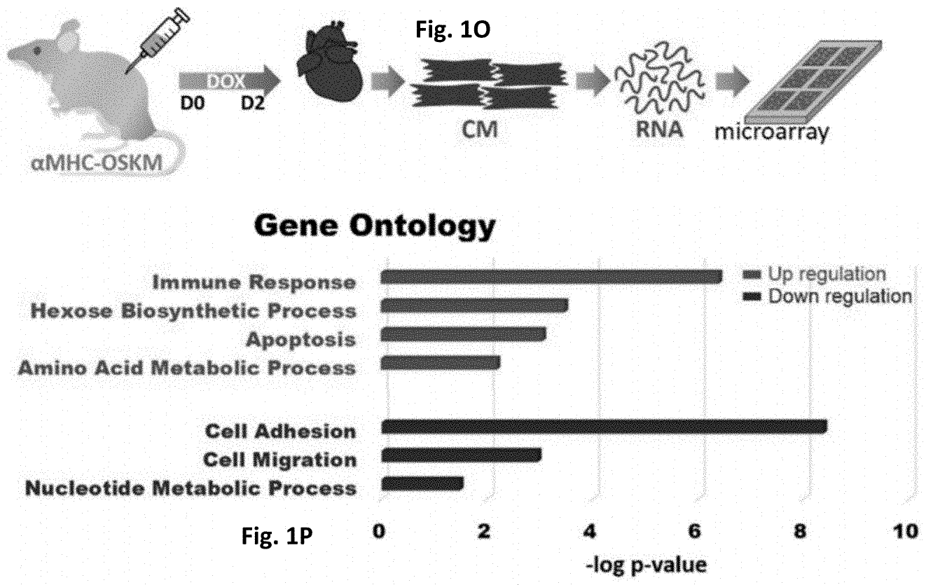

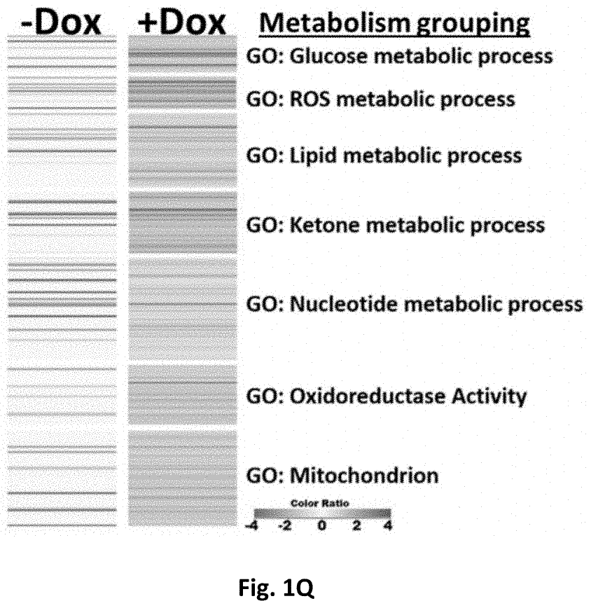

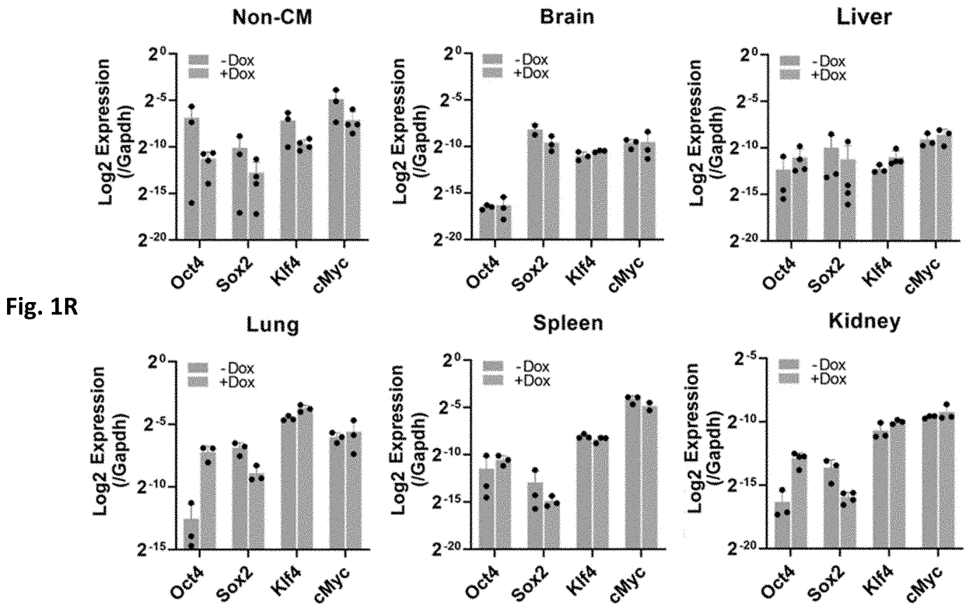

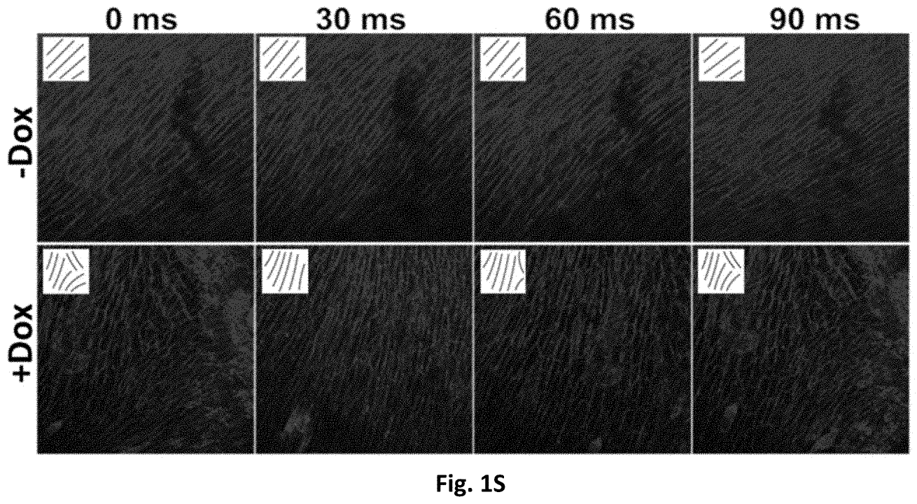

In order to examine the process of adult CM reprogramming in vivo, transgenic mice were generated to overexpress mouse OCT4, SOX2, KLF4, and c-MYC (OSKM) specifically in adult CMs after doxycycline induction as shown in FIG. 1A. FIG. 1B shows induction of OSKM mRNA expression in isolated transgenic, adult CMs after doxycycline treatment for 2 days. Importantly, this high level of induction was detected only in CMs but not other non-CMs in the heart or other tissues isolated from doxycycline-treated mice (FIG. 1R). Tracking the degree of CM proliferation by BrdU labeling, a three-fold in-crease in BrdU+ CMs was found 2 days following doxycycline administration (FIGS. 1C and 1D). The proliferative response of adult CMs was highest at reprogramming day 2 compared to day 1 and 4, and six days of doxycycline treatment was lethal. Therefore, reprogramming day 2 was selected as the key time point for further analysis. Using intravital microscopy to investigate the isolated whole hearts with membrane potential dye (Di-2-ANEPEQ) staining, we found that the alignment of CMs was changed after inducing re-programming for 2 days (FIG. 1E). Well-aligned CMs were observed throughout control (Ctrl) hearts, but regions of poorly-aligned CMs were observed in the doxycycline-treated mice (FIG. 1F). In addition, the in vivo morphology of reprogramming CMs was different from Ctrl CMs, maintaining their width but becoming shorter, leading to a different aspect ratio than control CMs (FIGS. 1G-1I). By recording each contraction of the Ctrl or reprogramming hearts in vivo using intravital microscopy, areas of disorganized or nonaligned contraction were observed consistent with the disruption of the normal aligned CM structure of the heart (FIG. 1S). Furthermore, heart tissue sectioning was performed to examine the relationship between CM alignment (WGA staining) and CM proliferation (H3P staining). We confirmed that the more proliferative CM population were found in doxycycline-induced hearts and these cells displayed a shortened morphology with poorer cell alignment (around 50-60 μm in length and an aspect ratio of approximately 3) (FIGS. 1J-1L). In addition, 2 times more Aurora b kinase (AURKB) positive CMs were shown in reprogramming hearts than in control hearts, showing that reprogramming CMs not only enter mitosis but completing cytokinesis (FIGS. 1M and 1N). Finally, in order to probe the mechanisms by which adult CMs dedifferentiate to regain their proliferative ability, CMs were isolated from the hearts of mice treated for 2 days with PBS or doxycycline, and RNA was extracted and subjected to microarray analysis (FIG. 1O). Gene Ontology data showed that metabolism-related gene expression was significantly changed in the reprogramming CMs compared to the Ctrl CMs at reprogramming day 2 (FIG. 1P). The gene expression changes included the up-regulation of glucose and amino acid metabolism and down-regulation of nucleotide metabolism. Similar trends were shown in heat map analysis; ketone metabolism-related gene expression was up-regulated and aerobic respiration-related genes were down-regulated in the adult reprogramming CMs compared to the Ctrl CMs (FIG. 1Q). Examining all of the data shown in FIGS. 1A-1S, temporary CM reprogramming induced dedifferentiation in the form of changes in cell morphology, proliferation, and changes in the expression of genes associated with metabolism.

### Example 2 Cardiac-Specific Ketogenesis Creates a Systemic and Specific Metabolic Switch Along with Mitochondrial Changes, Inducing CM Dedifferentiation at CM-Reprogramming Day 2

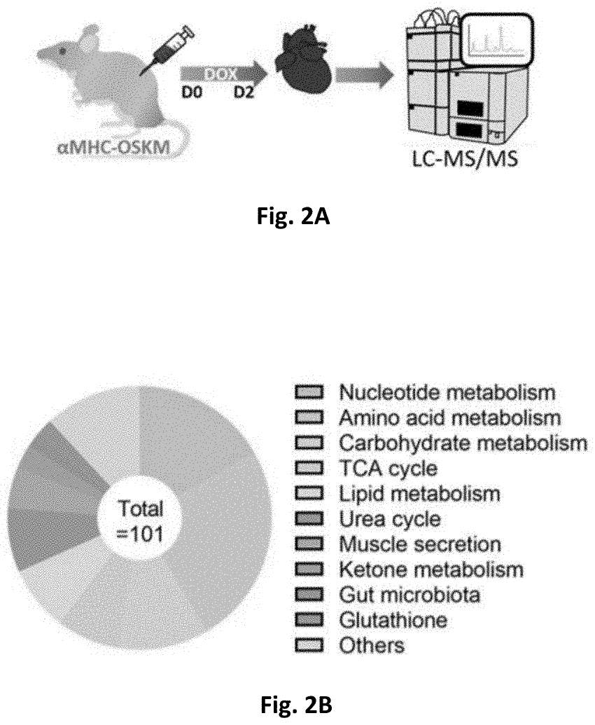

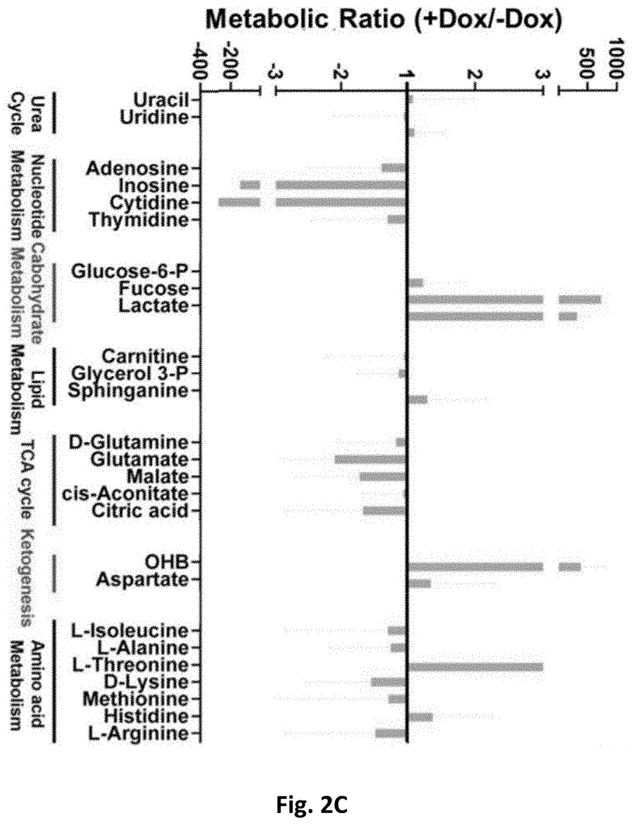

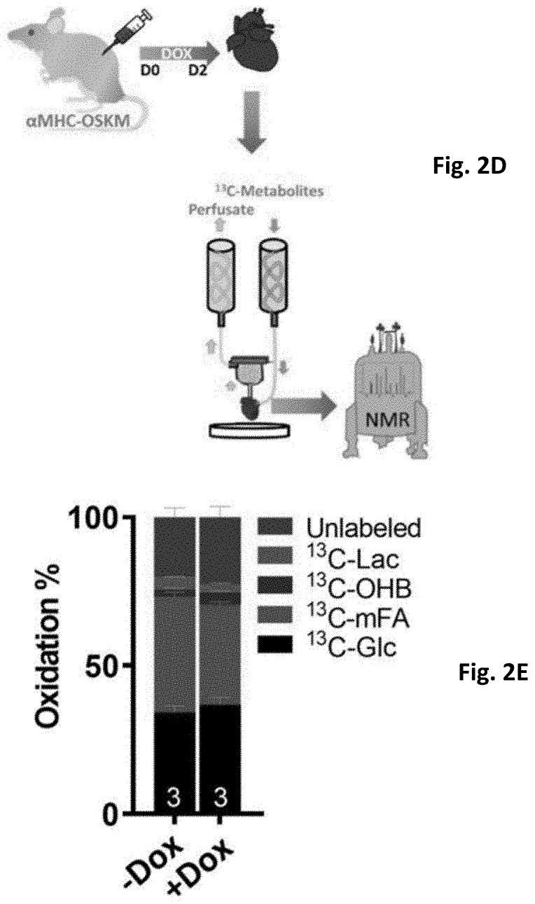

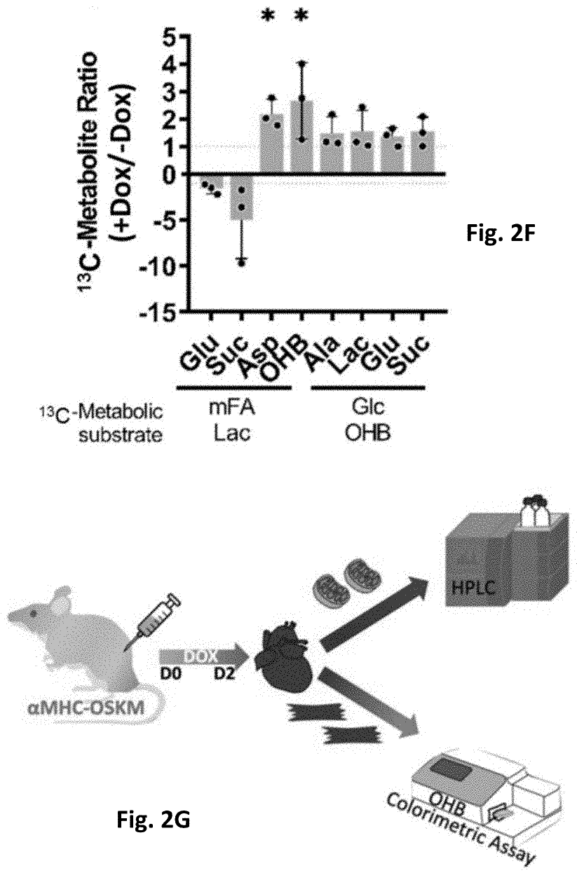

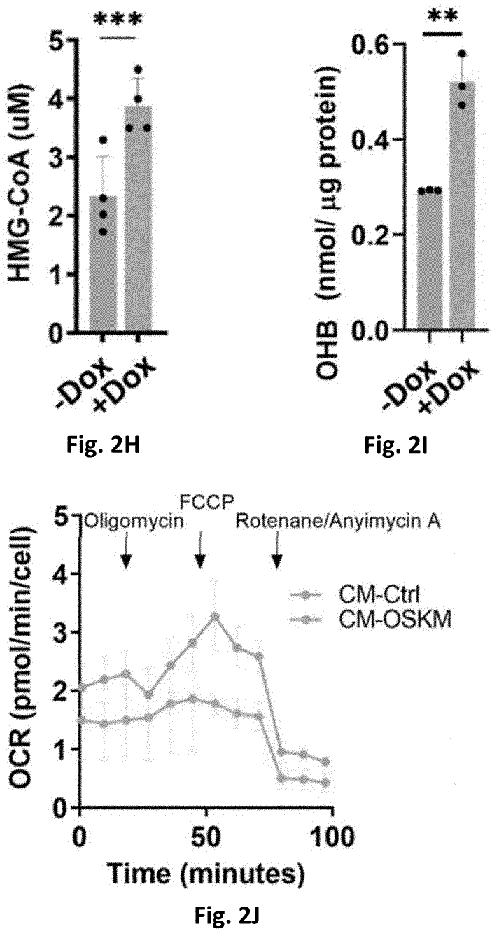

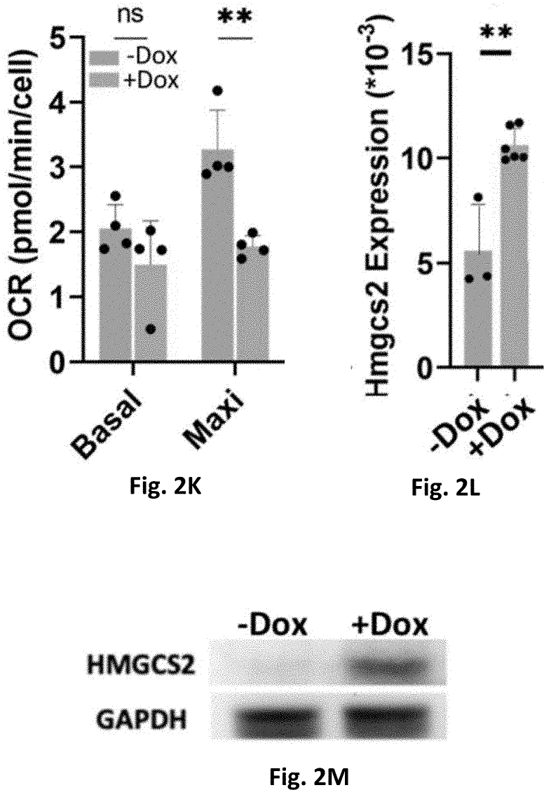

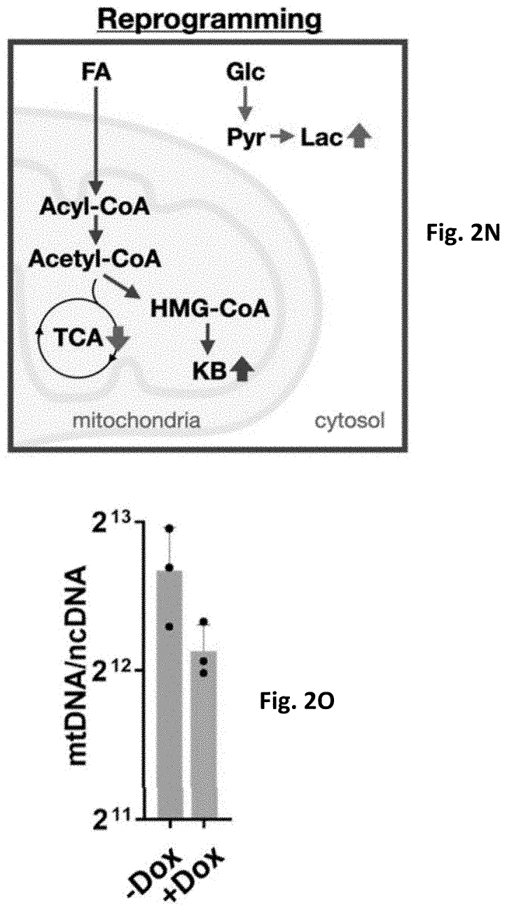

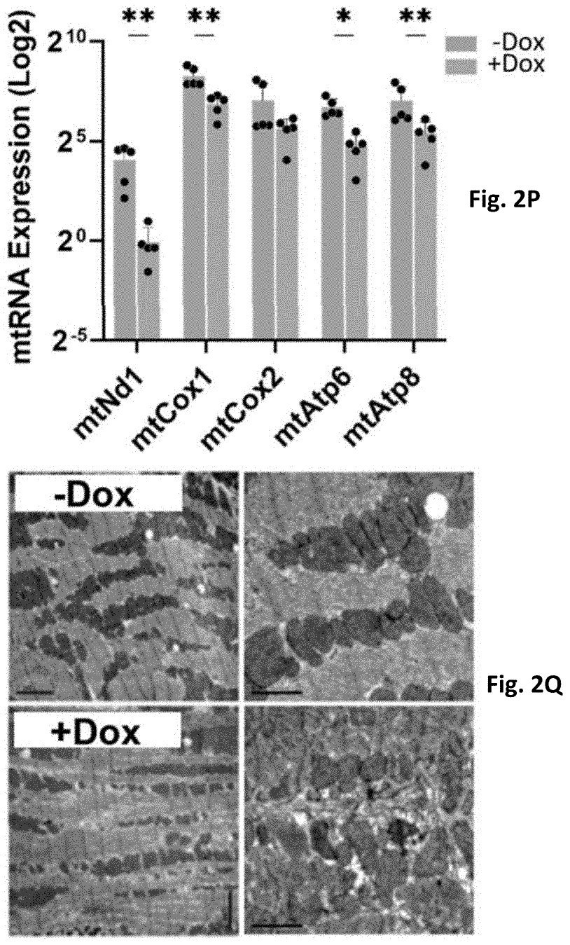

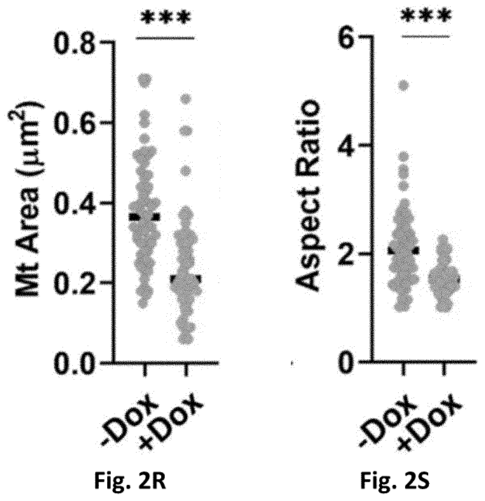

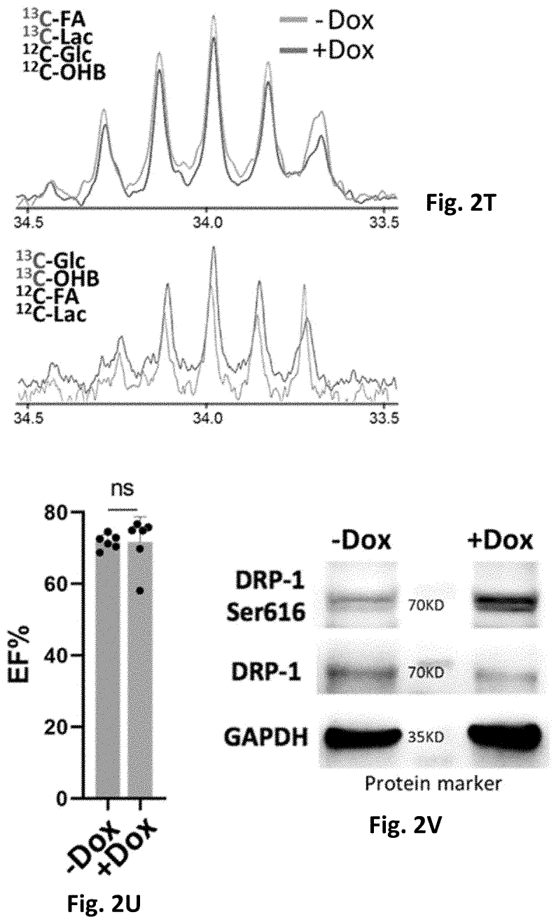

Since a metabolic switch appears to be intrinsically linked to adult CM dedifferentiation, it is necessary to clarify the detailed rearrangement of metabolic pathways in adult CMs which are undergoing reprogramming. First, the metabolic profiles of Ctrl and CM-reprogramming hearts were analyzed by liquid chromatography-mass spectrometry (LC-MS) metabolic profiling, and 101 metabolites were detected in both groups (FIGS. 2A and 2B). Grouping these hits revealed that glucose and ketone body metabolism-related metabolites were up-regulated in CM-reprogramming hearts (FIG. 2C). On the contrary, tricarboxylic acid (TCA) cycle and nucleotide metabolism-related metabolites were down-regulated in CM-reprogramming hearts which is consistent with the microarray data (FIGS. 2C and 1Q). In order to avoid influence by intermediate products derived from other tissues, a working heart system was set up and carbon NMR was used to detect the 13C-metabolites produced only from the exogenous addition of labeled substrates (Li et al., 2017; FIG. 2D). In NMR analysis, mixed fatty acids (FAs), which are the primary fuel for aerobic respiration, were decreased in the reprogramming hearts compared to the Ctrl hearts (FIG. 2E). Although glucose and ketones slightly increased for oxidation, the aerobic respiration derived from exogenous 13C-metabolites were decreased in the reprogramming hearts (FIGS. 2E and 2T). In addition, the amounts of Lactate (Lac) and Ala-nine (Ala) were 1.5-2 times higher in reprogramming hearts than in the Ctrl hearts, indicating that glycolysis (anaerobic respiration) was increased in the hearts two days following OKSM induction (FIG. 2F). Interestingly, both β-hydroxybutyrate (OHB, ketone) and Aspartate (Asp) were 2 times higher in the reprogramming hearts than in the Ctrl hearts, indicating that ketogenesis is increased (FIG. 2F). Since ketogenesis and the TCA cycle share the same metabolic substrate, Acetyl-CoA, ketogenesis induction should competitively reduce aerobic respiration in mitochondria. In order to confirm this concept, several techniques were utilized (FIG. 2G). The main intermediate product of ketogenesis is HMG-CoA. Therefore, we isolated mitochondria from Ctrl and reprogramming hearts and quantified HMG-CoA by high-pressure liquid chromatography (HPLC) (FIG. 2G). The amount of HMG-CoA was 2 times higher in the mitochondria isolated from reprogramming hearts than in the Ctrl hearts (FIG. 2H). The end product of ketogenesis, OHB, was measured by an OHB colorimetric assay kit. We found that OHB is more than 1.5 times higher in the reprogramming CMs than Ctrl CMs (FIG. 2I). Using the Sea-horse assay we found that the oxygen consumption rate (OCR) is lower in the adult reprogramming CMs than in the Ctrl CMs (FIGS. 2J and 2K). HMGCS2, the rate-limiting enzyme of ketogenesis, was up-regulated in adult reprogramming CMs compared to the Ctrl, as determined by microarray analysis. Moreover, the expression of HMGCS2 was significantly increased at both the RNA and protein levels (FIGS. 2L and 2M). A summary of these changes is shown in FIG. 2N. Interestingly, the changes associated with CM reprogramming did not affect overall heart function, as reprogramming hearts showed similar ejection fraction % (EF %) to Ctrl hearts (FIG. 2U). Several metabolic pathways such as ketogenesis and aerobic respiration are carried out in mitochondria, and changes of OCR are always accompanied by mitochondrial differences. Thus, CM mitochondria were assessed by measuring mitochondrial DNA content and mitochondrial RNA expression in the Ctrl and reprogramming CMs. The mitochondrial copy numbers were lower and RNA expression was significantly lower in the reprogramming CMs compared to the Ctrl CMs (FIGS. 2O and 2P), indicating immature mitochondria were shown in the reprogramming hearts. Transmission electron microscopy (TEM) revealed that mitochondrial area and aspect ratio were both significantly decreased in the reprogramming hearts (FIGS. 2Q-2S). Mitochondrial fission is reported to be related to proliferative induction through post-translational phosphorylation of DRP-1 on serine 616 (Marsboom et al., 2012). Indeed, DRP-1 serine 616 phosphorylation was higher in reprogramming CMs compared to the Ctrl CMs (FIG. 2V). These data indicate that during CM reprogramming by OSKM induction, a metabolic switch occurs, including increased ketogenesis and glycolysis and deceased aerobic respiration with immature mitochondrial structure and function. This switch occurs in synchrony with the induction of CM proliferation.

### Example 3—Forced HMGCS2 Overexpression Effected Before Myocadial Infarction Increases Adult CM Dedifferentiation and Proliferation for Heart Function Improvement after Myocardial Infarction or Under Hypoxia

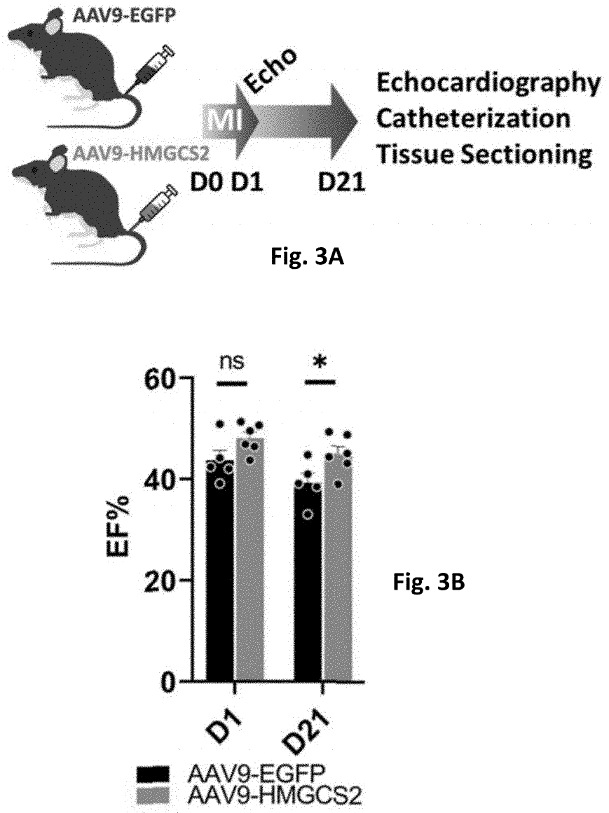

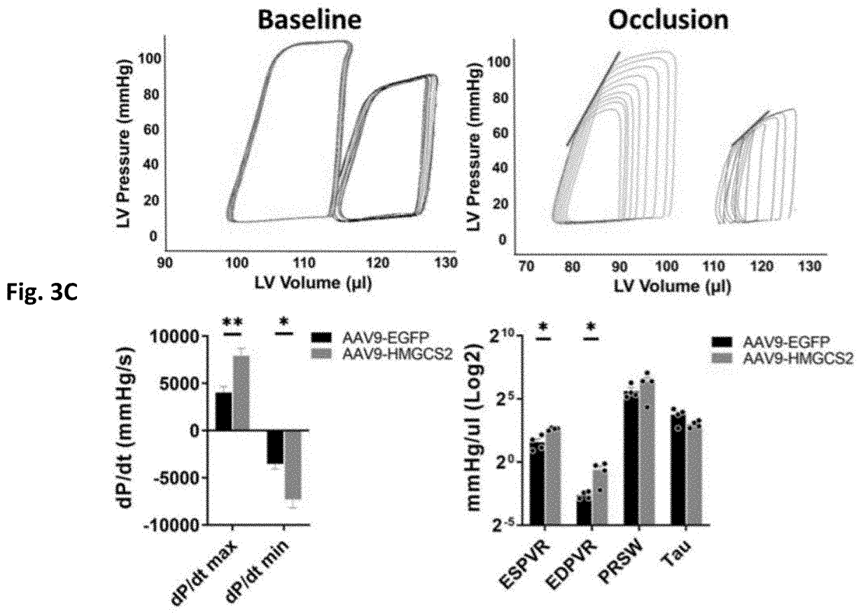

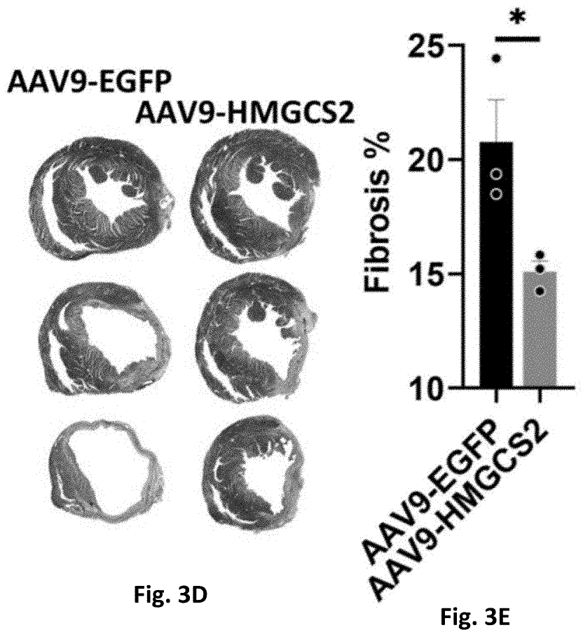

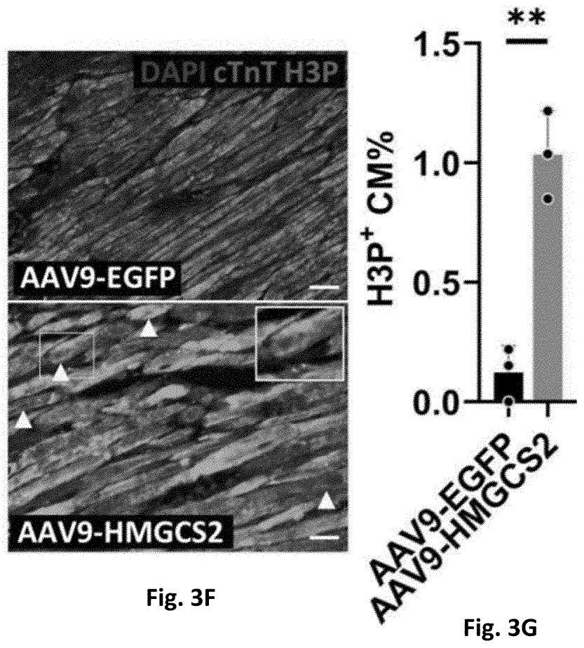

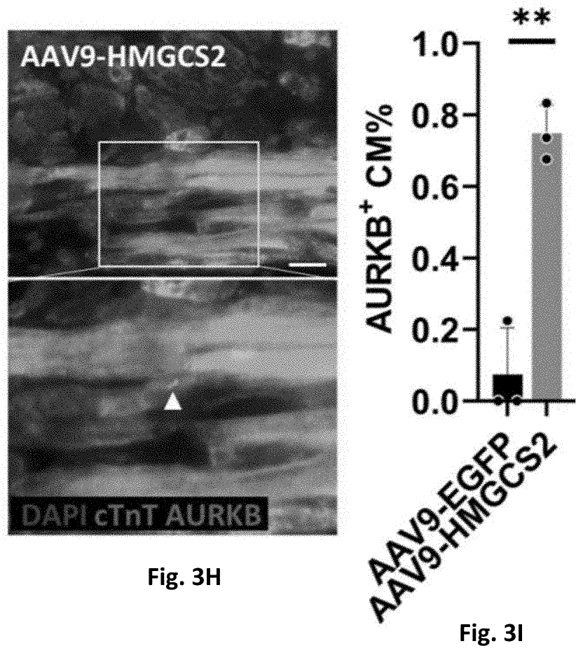

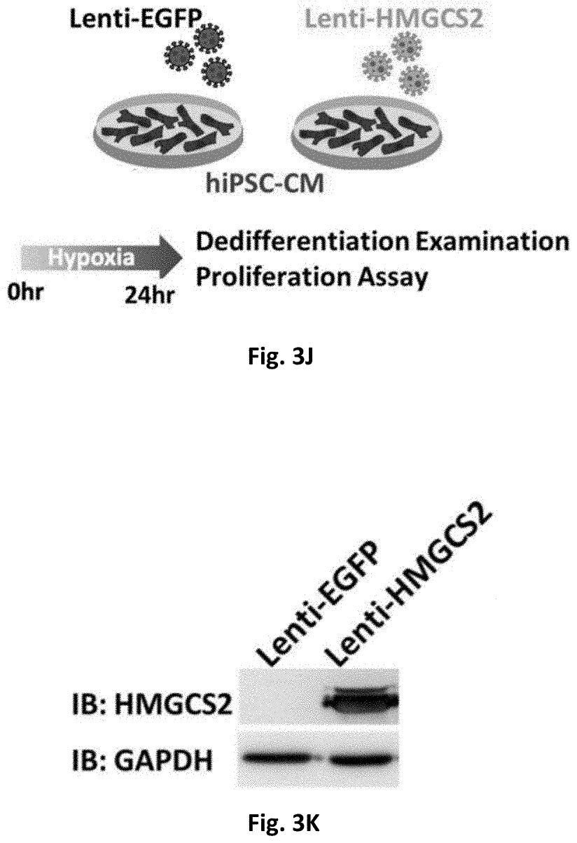

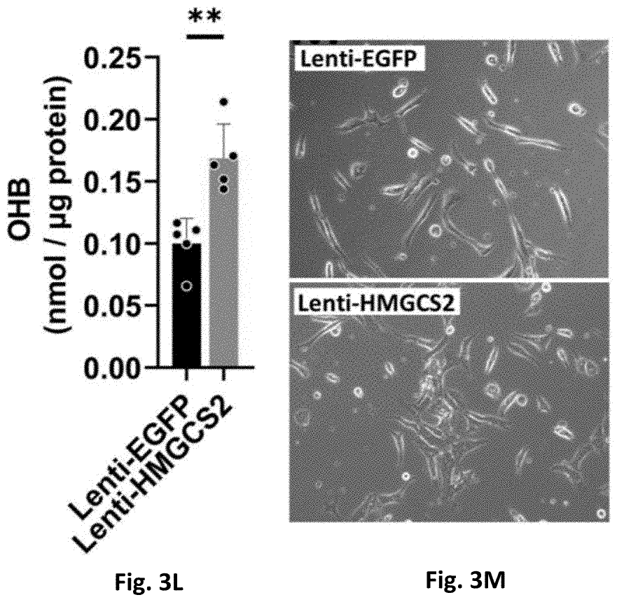

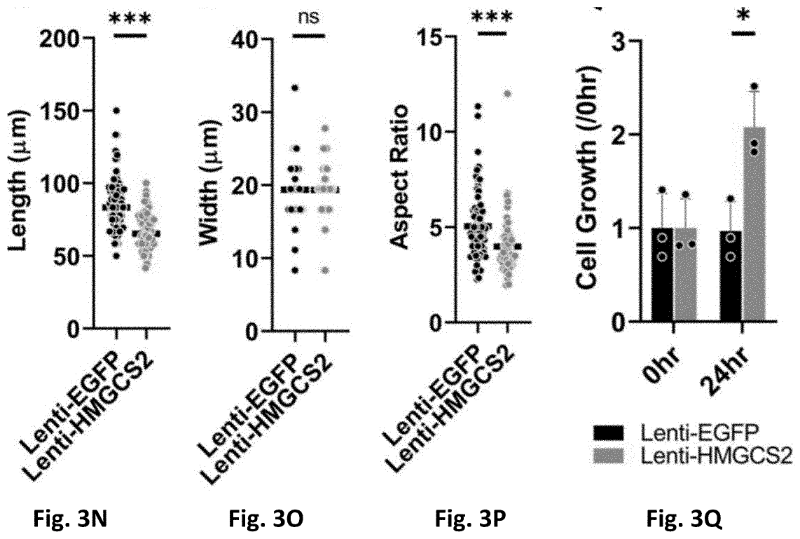

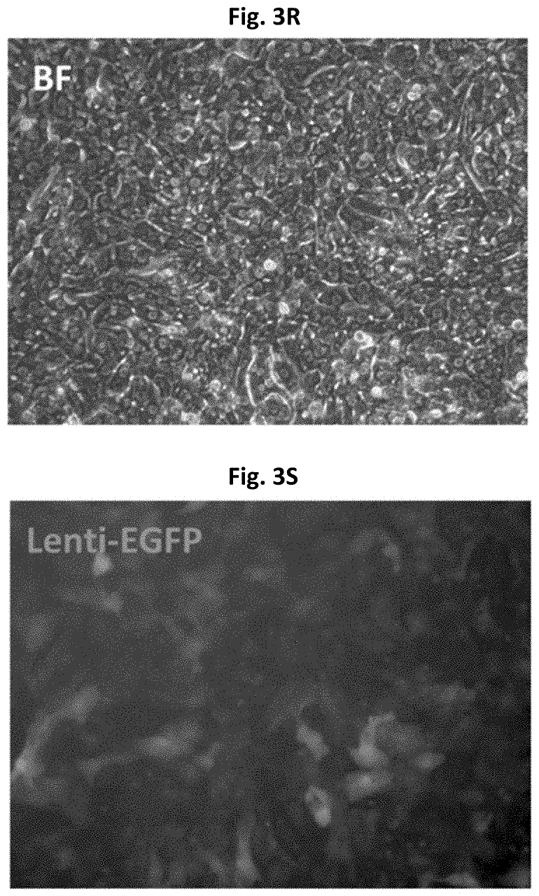

In this section, we aimed to investigate the possible therapeutic role of HMGCS2 on a permanent coronary artery ligation myocardial infarction (MI) model (FIG. 3A). After exogenous HMGCS2 induction by AAV9 induction for 5 weeks, HMGCS2-overexpressing mice showed a higher EF % at D21 following MI surgery than Ctrl AAV9-EGFP mice measured by echocardiography (FIG. 3B). Catheter measurements indicated better heart function in HMGCS2-overexpressing mice 21 days after MI injury compared to Ctrl mice (FIG. 3C). The fibrotic area was also smaller in HMGCS2-overexpressing mice compared to the Ctrl mice (FIG. 3D, E). More H3P+ and AURKB+ CMs were found in HMGCS2-overexpressing hearts 3 days after MI injury compared to the Ctrl (FIGS. 3F-3I). Taken together, these findings show that exogenous HMGCS2 expression can support cardiac regeneration and improve heart function after MI. Next, we examined whether these findings could be replicated in an in vitro model using hypoxic human induced pluripotent stem cell-derived CMs (hiPSC-CMs) (FIG. 3J). HMGCS2 expression was highly up-regulated in hiPSC-CMs after lentiviral infection (Lenti-HMGCS2) compared to the Ctrl (Lenti-EGFP) (FIGS. 3K, 3R and 3S). HMGCS2 overexpression also induces increased ketone production in hiPSC-CMs (FIG. 3L). Furthermore, HMGCS2 overexpressing hiPSC-CMs showed a shorter morphology with a lower length-to-width ratio compared to the Ctrl cells under hypoxia (FIGS. 3M-3P). This shows that HMGCS2 overexpression supports human CM dedifferentiation, as we found in adult mouse CMs shown in FIG. 1. Finally, HMGCS2 overexpressing hiPSC-CMs showed a two-fold greater proliferative ability compared to Ctrl cells under hypoxic conditions (FIG. 3Q). These data indicate that forced HMGCS2 overexpression supports CM dedifferentiation and facilitates proliferation under hypoxic conditions.

### Example 4—Forced HMGCS2 Overexpression Effected after Myocardial Infarction Increases Adult CM Dedifferentiation and Proliferation for Heart Function Improvement after Myocardial Infarction

In order to test the possible therapeutic role of HMGCS2 on heart regeneration, exogenous HMGCS2 was induced immediately after performing a permanent coronary artery ligation myocardial infarction (MI) model (FIG. 4A). After exogenous HMGCS2 induction by intramyocardial AAV9 injection immediately after MI, HMGCS2-overexpressing mice showed a higher EF % at post-MI D21 than Ctrl AAV9-EGFP mice (FIG. 4B). Catheter measurements indicated better heart function in HMGCS2-overexpressing mice 21 days after MI injury compared to Ctrl mice (FIG. 4C). The infarct area showed no differences in Ctrl or HMGCS2-overexpressing mice 1 day after MI (FIGS. 4D and 4E), indicating that HMGCS2 overexpression may stimulate regeneration rather than protecting the myocardium. The fibrotic area was also smaller in HMGCS2-overexpressing mice compared to the Ctrl mice (FIGS. 4F and 4G). More H3P+ CMs were found in HMGCS2-overexpressing hearts 3 days after MI injury compared to controls (FIGS. 4H and 4I). Taken together, these findings show that exogenous HMGCS2 expression can support cardiac regeneration and improve heart function after MI.

### DISCUSSION

Adult CMs undergoing early OSKM-induced reprogramming display metabolic changes which allow for enhanced dedifferentiation and proliferation in vivo (FIGS. 1A to 1S). Our previous study investigating early-stage neonatal CM reprogramming in vitro found up-regulation of proliferation-related gene expression (Cheng et al., 2017). However, neonatal and adult CMs differ significantly in their structure, function, metabolism and response to injury (Szibor et al., 2014). In addition, the gene cocktail described in our previous study was unable to efficiently induce proliferation in adult CMs. This indicates that adult CMs and neonatal CMs induce reprogramming via different mechanisms. These data suggest that inducing a metabolic switch of adult CMs, rather than directly inducing cell cycle-related activators, may be a more efficient way for giving rise to the cellular phenotype adaptations necessary to regain proliferative ability (FIGS. 1A to 1S and FIGS. 2A to 2V). Since adult CMs are notoriously difficult to maintain in culture, and the reprogramming process may be affected by the cellular microenvironment, this study profiled the changes which reprogrammed adult CMs undergo in vivo. Through specific induction of adult CM reprogramming in vivo, we not only can investigate the transformation of CMs during the process, but its effects on whole mouse can be also detected. This system undoubtedly is a powerful tool to study the reprogramming process specifically at the tissue level in vivo and to explore how reprogramming of specific tissues has systemic effects.

Ketogenesis is mainly carried out in liver tissues, where ketones, as water-soluble metabolites, can be easily transferred to other tissues for utilization (Grabacka et al., 2016). Ketone utilization is common as an alternative energy source while fasting or exercising (Puchalska et al., 2017), and ketones are also reported as the preferred metabolic substrate for heart improvement after injury (Anbert et al., 2016; Horton et al., 2019; Nielsen et al., 2019). However, there are few studies clearly defining the role of ketone synthesis in the heart tissue itself. Here, we demonstrate that HMGCS2-induced ketogenesis in adult CMs competitively reduces FA metabolism leading to a metabolic switch and mitochondrial changes (FIGS. 2A to 2V). Metabolic flexibility allows cells to adapt certain conditions, and primarily occurs due to the antagonism between glucose and FA for providing energy production (Bret., 2017). Besides, ketogenesis plays as a critical regulator to control FA metabolism, Glc metabolism, and TCA cycle for maintaining hepatic metabolic homeostasis (Cotter et al., 2017). The same scenario is presented in our current study, showing that an increase of HMGCS2-induced ketogenesis in adult CMs decreases FA metabolism, and glucose is then used via anaerobic or aerobic respiration, based on the available oxygen. Therefore, ketogenesis-induced adult CM reprogramming can be specifically induced in the border zone but not the remote area of injury hearts.

HMGCS2 is up-regulated in the mouse heart ventricle within one week after birth, and its expression is diminished at postnatal day 12 (Talman et al., 2018). However, the role of HMGCS2 in heart function maintenance during development or after injury had not yet been shown. Under certain condition such as reprogramming or injury, exogenous HMGCS2 expression increases adult CM dedifferentiation and proliferation. All these data suggest that HMGCS2 may not be a driver but is required for starting adult CM dedifferentiation and proliferation, and this requirement successfully supports cardiac protection and regeneration after injury (FIGS. 3A to 3S and FIGS. 4A to 4I). In previous studies, genes responsible for proliferation such as OSKM always carry a risk of tumor formation, which limits therapeutic applicability (Ohmishi et al., 2014). However, HMGCS2 controls the metabolic flexibility, allowing adult CM dedifferentiation and proliferation during cell stress, thus providing an ideal therapeutic target for heart diseases.

Overall, this is the first study to perform and investigate OSKM reprogramming specifically on adult CMs in vivo. We have demonstrated the importance of HMGCS2-induced keto-genesis as a means to regulate metabolic response to CM injury, thus allowing cell dedifferentiation and proliferation as a regenerative response. Furthermore, overlaps between OSKM-induced CM reprogramming, heart development and maturation, and the response to heart injury become readily apparent. Since myocardial infarction remains the greatest cause of death in developed countries, we hope this study provides a foundation for future research, exploiting metabolism as a mechanism to drive myocardial regeneration following injury.

| Sequence Listing | |
|---|---|
| SEQ. ID NO.: 1 | |
| 1 | aggactcctc cctcaccaaa ctctgcaggctttgaaatca aagttctaaa tgtctcccca |
| 61 | ggcaatcaga aaaggcaaga cctggcaaat aagaggttgt actaaccagt aacaaaatca |
| 121 | caaacaacat ttgctcttcc tcttccacag cagactccac aagtaggtgc aatgaaagag |
| 181 | ccctagattt ggagccaagg ccgtcaaatg ccctcccagc cattgtcact aatcacatat |
| 241 | ccacaagcca gatcacttaa tctctcaaag cctcaatgtc catatcttcc aaatggggct |
| 301 | aataattcag gttaactcca tggaactttc atgagaaaag accgtatgca aaagcaactg |
| 361 | aaaactgata aagcaccaga tatgctagta atgcattagt atcgtgaaat aaacagggct |
| 421 | catttccaaa ggtacaaaga ccctgcaagt ataaagactt cttcctaggt ctagactttc |
| 481 | catagaaata gctttcctac ccactttctg atgccgagaa ttttgaaagt tctttttccc |
| 541 | ttaggttgag atgtaaaggg caaatctgca tgggaaaaga ttgcttcaat ttatcagtca |
| 601 | tgggaacctg gggtaaatgc attttcagag catttattga aaggagaata gtgggctact |
| 661 | gaggtagaag agttgcaatc tttatgtggg ctaaaagagg caaatccagg tgcctgggaa |
| 721 | ccttgtttat agttttgttc tcctacaccg gctcttttgt cagaattgct taaaaaacaa |
| 781 | acattgtttt tgcaagacct caccctagat gtctaaactt ctaaaatccc tcataatcaa |
| 841 | tttttctgac ttttaatgct tatctagcag gtaacatgca ttttaaatta atccttttat |
| 901 | caacacttca gctgaaaagc tgaagtctag gagttgaagg accctaaagt ctcaaatcaa |
| 961 | aaataaatac atcttttttc atctaggaag tatcaaaatg tgggtttatt taagtatttg |
| 1021 | ggaggtagta tcttcttcag acacaaatag tgtgttccat tttcttcaac actttgagca |
| 1081 | attagtagac aaaccagtta tttgattgta tttgaataca attacttgac taagtcatat |
| 1141 | aaatttcctt cagtatgaaa aactaccacc tcatggtgtt ttactattat ttccctcaat |
| 1201 | ttatactttg cataatgcat tcctggtgct tcctcaatct acaagttccc ttatcccaaa |
| 1261 | ggaacaactt aatattagat tggccatata aaatttccac cttcccaagt caaaaatggt |
| 1321 | tcatgattga ctcaggttat gtgtagagcc agatacctgg attcaaagcc cattcaggcc |
| 1381 | atttactaga tctaaaacca caaatggtta tataattttc ctgaacctca gtttcctcat |
| 1441 | ctgtaaaatg ggcttaacaa tagtgccaac ctcagacagt tgtaaaaatt aaatgagata |
| 1501 | atgaacggaa agtacttagc acagtaccta gcacgcagta attacttagt acatgtcagc |
| 1561 | ttaaaagaga gaagggaatg aagttgatcc atctatctgt attcccagtg cttatcacag |
| 1621 | tgccaatttg ttatatacac taattaaaat ttgcattgga ttggatagtt ttggtcttca |
| 1681 | attctatcaa actgagccat gatgtagcca taatcccgtg tgatgtttgt gtaagagttt |
| 1741 | aatgtttcta ttgttaaaag taaaaccttg aacaaattaa atttagttga atttatttga |
| 1801 | gcaaagaaac cattcatgaa taagtcagca ccctgaatta gtaaagattt agagatctcc |
| 1861 | aatagaaata ttggactgtc agtatttaga gacaaaaata gcttgattgg ttacagctgg |
| 1921 | catttgcctt acaggaacat gttttggcaa tttgcagcct gcgattgact gaaagcatgg |
| 1981 | ctgctatgat tggtcaagac tcagctactt gttacatgaa tacactctca ggttaggttg |
| 2041 | cggtttgttt atatattagg ttaagtaccc tacctaccta ggcagttttg ggccacatta |
| 2101 | aatttacttt aacactatcg agttttatcc attttcttag tggaataagg aacatgtgga |
| 2161 | gactacctga gtactccaaa atttagagat cagaaagagg ggagcacctg tggggagtgg |
| 2221 | ccagggattt ggaggaaaac catgggattg tcaggtctaa gggcaaagtg aaaaaggtgc |
| 2281 | tttgagaaga agggagagca gccttgccat ttgctgctaa gaggtctagt aagctgaagg |
| 2341 | ttcaagagca aacactgcat ttggcaataa ggaagccact ggtgaccttg atgagaggga |
| 2401 | attccttgga gcactggggg caaaagcctt agtggtcaat taaagacaga atgagaggta |
| 2461 | agcttgtaaa acactgaaag cagacatttt aaataaattt ccctatagat gacagcatag |
| 2521 | atttggtggc actcagtaag acatatagag tcaagaggag gtatttaaag atgggattgt |
| 2581 | ataagaacaa taacacaaga agaatgtagt agaaagggaa aatagatcat gagaaagaga |
| 2641 | ggggaaaact gcaggagcac agccttatgt gagaaacaag cagtacaacc agtgcacaca |
| 2701 | tggtggtgct ggcccgaggc cagagcaggg actcttcctc tgcatagtga gaaggcagtg |
| 2761 | agaaggcaat gtgtggggta taaaggcagg caatttgcca gatttgctca tggaaaacgg |
| 2821 | agttattctt ttctgattgt ttctattttc tcagtgaatt cagagtcaaa gtgatcagct |
| 2881 | gagaatgagt agaaaggggc tatggcagaa gagaagttgt gactagccct cttgggatgg |
| 2941 | gagagcaaat ggactgggaa aaggtagtag agttaccagg ccatggtgag ggtccacttg |
| 3001 | agatgtatgt ttgtaaattt aaagctaaca agttagtaca aagttgtgtt tttcttcatc |
| 3061 | tatgtttagc tgctcagatg caggcgcaga gtagattaag agttgggttt aaccaaaatt |
| 3121 | gaaggtttgc taggccagtc cgacagagag cacaaattgc aaagtgtgtg caagggattg |
| 3181 | cttatggtga ggcaccatgg ttaatctgat ctggataagg agagaaaaga ggtgatgagg |
| 3241 | tgtaacaaat gctaaaaaca tagaggagtc agtggtggtc tcagtgggag aaaaaggtgt |
| 3301 | gagggtactt taaacaggag cagggaatat agaggtggtt ggaaattgga atgcatgaaa |
| 3361 | ctaaaatgtt ggaggtggca tagacactgt aataacaaag tccacattat gactgtggag |
| 3421 | tgggaggcta aagtcatgtc catgaacacg gacatggctg tgggagcttc agtgagaggc |
| 3481 | tagggcaggt gaattatctt atgtggagat tgacatctca catccattga gatgactgat |
| 3541 | ggtggagagg aaagtagtga tctatgtgct taaattttat caatgaggga cagtggaaca |
| 3601 | tgacagttag ttgactgcaa gaataagggc actgggtggc acagactgta gcatgtgctt |
| 3661 | taagacagca ggggttttga gaggaggaag aggagaaata ccctggaaga gatagtatga |
| 3721 | agcaaagagg acacaggcct tgttgtaagc atttaaagtg cattggattg acaagaagca |
| 3781 | acaggtattt cagagaagag attggaaatg aagaatttca ctgacgacag aactttgcaa |
| 3841 | aaggctgagt gtaagagcag gaggtgacac agcaaggtag gagattgagt tagaacacca |
| 3901 | acacaccaag atatatggag ataagaattt aaagataaga tggaagacct agatatcctg |
| 3961 | gactgctggg ggcacctaga cattctctgt ctgtaggaac atgaagtcaa ctatatcctc |
| 4021 | ctaaagcagg aaccactcca gctcttttct gtgttcccta cacactcata ttagacaggg |
| 4081 | ggttggtgat ggggatgact gaatgaacta aggagtgaat gcatgactca caaaaggtag |
| 4141 | aggaagattg gtgctgtggg aggagtggag ggagacattc atttggaaaa tcagatggca |
| 4201 | ggagcctttt tattgagtag tagaagagca aagcagaagg accagaatct ggagtcaaaa |
| 4261 | gacatggttg agttctcatt ctgcaacttc ctagctgcag gtctttggga aaatgactcc |
| 4321 | tttatacata actctgacct catctataaa gtaaaccttt cctccttagg agattgagct |
| 4381 | tcaaactgtc acctctttga ggctcctgtc cctttactac aacactaatt tcatcccact |
| 4441 | tggaaattgt gtagagctgt acaagtaaaa gggtggacaa taaacaggag aaatatagta |
| 4501 | ggttccacat actctaacgc ccagcccttg gcctatgtgc caacactcac tcccaactcc |
| 4561 | ttgaaaagct actattaaaa gagtttccct ttggtttaga aagatgtttc ttataatgca |
| 4621 | tagcacatta aaataataac aactaacacc acagagagga gtgtggaaca cccagtgaga |
| 4681 | gtaatacaga taaggagcca gggtctaaaa caagacacat agggttacct tgggatgtga |
| 4741 | tacaacaagg aacatcataa cctcctgctt aggtagctgg gcagaatcaa ggctgccaca |
| 4801 | gagcctgatg gagtaggagg aacaatgccc agccattccc acacatgctc aggagcaggg |
| 4861 | cagctatgta catgttggag agatgctgtt tgtctttgac tcgcccgtgt tctgagtgag |
| 4921 | ccctttgacc cagttttaga agcagactga gccacggtga gcagaggcgg ggcttaggga |
| 4981 | ggcaggagtc ttggggcttt ataaagtcct gccgggcacc actgggcatc tctttcaagg |
| 5041 | tttctgctgg gtttctgaac tgctgggttt ctgcttgctc ctctggagat gcagcgtctg |
| 5101 | ttgactccag tgaagcgcat tctgcaactg acaagagcgg tgcaggaaac ctccctcaca |
| 5161 | cctgctcgcc tgctcccagt agcccaccaa aggtgagtca ctttctgaga agcaccttgt |
| 5221 | aactagtaaa agatagtttt tccctgctat tggggaaaac tcactagaat cccactcaaa |
| 5281 | atttggcaag gcttgtgcac agcagcctta gacaagcaag ttaactttaa agggtctcag |
| 5341 | ttacctcatc tctaaacaga caatcccttt cagctgtaga gtgagaagag cccaaacctc |
| 5401 | tgacacatgc tgtgtttgtg agcaatggca acttttactc tgccagctgc atgaagcagt |
| 5461 | agaaatatca gtaccaggcc acagctttcc tctctacacc accattccca ccttcacccc |
| 5521 | tagcctctgc ctagaaccac aggaccttgt gccaactgca gtgttagtaa aaccagtgac |
| 5581 | tttatatcac tgcagcagaa tcagaaatgg actgaggatg agaagctgtg tttgccttgt |
| 5641 | gttccaattt tatgaaaagg ggaaatgtgt gtttatgtgt gtatgtgtac atgctctttg |
| 5701 | caagaagaac atgcacactc cttttctttg taaatagtcc ctgaacatgg ctcaagtgct |
| 5761 | tatgttttcc attgtcagcg atgatggtaa cacagctatc gttagtgcct caggctccca |
| 5821 | gccacctatg tgtttctgtc taatccccaa accatccact acacattggg actagttctt |
| 5881 | tatttcctta catttttact ctatattcta tgactactaa atatttagaa aaatgatttt |
| 5941 | gacctagtgt ctttccttgc caaataccca aggaacctgg gtgtatagat gtgcatggta |
| 6001 | gaggcaaatg cacatagctt tcttatattt ttcattatgc taccatcatc tcactctccc |
| 6061 | catgcactgc caaaccctgc atgtgggtta aatgtcccag ctcaggattt aacctgtttc |
| 6121 | tatatttgtg aagaagagat tgatgtgggt ttcttgtttt aatagcaata gttggccatc |
| 6181 | agccaaaaga catacatcaa tcctccccaa cattctgact cccttggttc aaactcttgg |
| 6241 | aatcattccc atttcccttc tggtatattc acagttaatc ccattatgca tggcttgaac |
| 6301 | taatattgct tttcatgagt caccttttct ctatatgtct aatcgccttt aatccaaccc |
| 6361 | acattggctc taactccaac ctaaaagaga ctttcatctc agcatctgct ttgctgtctt |
| 6421 | caaaattcgg taagacttgt gccctccact tactgtattt ctcacatatt gtctccctgc |
| 6481 | tcccctatac acctgcatct ccagggttcc ttacttgttc agtcaccccc tgccgtggcc |
| 6541 | actgcccctt cattcccctc cagttcttca ctggcagaag tctgtcatcc atcaaggttt |
| 6601 | gcctcaaatg cggtctcttc cacgaagctt ctctgatcct ccaacccact gaaatctctg |
| 6661 | cttcctttga actcctgtag attttgctca cattcctttt tgtgggcctg accacattct |
| 6721 | gccttgaagt tgggttatat gtgtgcttat cattcctcac actggtcaag gaggtctcaa |
| 6781 | gaacctcacc ctcttctttt ctttgtccaa cccctttgtt caaccctcac aaacccttcc |
| 6841 | cagcacagtg cctgaagtgt agtaattaat tttgaaacac aagggaagga ggcaagatgg |
| 6901 | aatacagaag taaaggtgtg gtgcatgttc ttgaagtggg caacaccagg agaaaaatga |
| 6961 | tttaaaatta cacaaagtga tcattcttta gagaaagcac aagatgagaa ggatactctt |
| 7021 | aacttcggtg ggctgaagct tctggaagcc tctccgtgtt aattttcttc aaggctttat |
| 7081 | aatccatttc tagaaatagc tccccaccaa gacagctaca aaagttacca actgacccat |
| 7141 | tctaagcttc ttcttgcaag ctttgatttc taactgggaa gaaagggagg gagccagccc |
| 7201 | agagaagtca gagcgagaat gaggctgaga gaaaggcagc caagctggca ggacaagcgc |
| 7261 | tggcttaacc attagctccc gggtactggg gaagctcctc cgtaaatatt tgagagtaca |
| 7321 | aactccagtt atttggaggg agtcaaataa atagggaaga taaataaact ccaaacctct |
| 7381 | cctgtcagat ataatgtgta tttatcattc tgcctcacta tcttgtgatc atatgatcca |
| 7441 | cttttgcctc acagctgtcc ttagaagtga ccttgctgct gggagaggct ctagaattct |
| 7501 | accagaggct cagaatccca aagatgattg atagacacat tcaatctgag ttccagctcc |
| 7561 | cgtagaatgg agctaaattt ataagcctgg cacccagggc agtgaaggga cagagtattt |
| 7621 | ctaacacgtg agaaactatg aagttaccct gagtgcatca ctttaccagt gtgtgccttg |
| 7681 | gtttcactaa ctataaaatg aagaatgttg ctaaagtgaa cagaaggtat aaagtacttt |
| 7741 | tgtatgggag cagtacagag atcaccaagt tcacctccag tatgctccca tacaaaaggg |
| 7801 | aacacagatt ttcgccaggg atattaagaa tctgggttaa agagaagtga attggtccag |
| 7861 | aaaagaaata gatcatctct cccttcttct gctgactcct tccccttcct tttttcctct |
| 7921 | gctctcgttt agaattgctc tttctgctgt ctgtgttccc tgcatattta gctgtaaaat |
| 7981 | gtctgcttct ttcactgggc tgtgctctct ttatgggcac aatgcatgtc ttattcactg |
| 8041 | ctgtgtattt ggactagaac tgtgttgggt gtgctcaata aacattggaa ggccctatca |
| 8101 | gaaaaatcag ctagcagaaa acttacttaa aagtaggaaa acagtgggta tgttcttgtg |
| 8161 | tagaaaaaag aaaggagaaa gacatgtaat tagaggtaca cttttaaaat gagtaaagat |
| 8221 | tgtataatta tgccctataa gggcttataa catgtagaag taaagtatat gacaataatg |
| 8281 | gttcaaaagg atgcagagag taaataaagt caacctaaag tttttgcagt gttccaaaag |
| 8341 | taagataagt attaatttaa gtaagattac aacaagccaa ttatgcatgt tataatcttt |
| 8401 | aaggtcacca gtaaaaggaa aagagggtat aaaatgaata ataaatattt gcttactcta |
| 8461 | aaaggatatg ggaaaggagg aataaaagaa caaagaacaa atgagacaaa tagaaacaaa |
| 8521 | taaaaaaata gacttagttc cggctgggcg tggtggctca cgcctgtaat cccagcactt |
| 8581 | tggaagaccg agatcaggag atcgagacca tcctggctaa cacggtgaaa ccctgtctct |
| 8641 | actaaaaata caaaaaatta gctgggcgtg gtggcaggtg cctgtagtcc cagctactca |
| 8701 | ggaggctgag gcaggagaat ggtgtgaacc caggaggcgg agcttgcagt gagcagagat |
| 8761 | cacgccactg cactccagct tgggtgacag agtgagactc cgggtgacag agtgagactc |
| 8821 | cgtctcaaaa aagagaaaaa aaaagattta gttccaacta tattagtaat tacaacaaat |
| 8881 | ataaatggat gaaatactca aattaaaaca ccactattgt tagacttatt aaaatttttt |
| 8941 | taaaggacta aaatatatat accgatcaca agagatgtat gttaaagata aagacgttaa |
| 9001 | gaagttgaaa gtaaaaaggg acagaaaaag atgtaccatg gaaacagtaa gcaaaaagct |
| 9061 | agtgtagcta tatcgacgtc aggaaaggaa actttatgcc aagaatatca caaagatgaa |
| 9121 | aagggatatt taataagtat agaagggtca attcaatgaa aagataataa caatactaaa |
| 9181 | tttgtagtca tctgataaca tagcttcaaa atatagaaaa ttaattaaat gattgctatg |
| 9241 | ttactgtctt ttgaggaaat tgtctacaga ccattagtgg gagtttgact gttatctcca |
| 9301 | tcacaggttt tctacagcct ctgctgtccc cctggccaaa acagatactt ggccaaagga |
| 9361 | cgtgggcatc ctggccctgg aggtctactt cccagcccaa tatgtggacc aaactgacct |
| 9421 | ggagaagtat aacaatgtgg aagcaggaaa gtatacagtg ggcttgggcc agacccgtat |
| 9481 | gggcttctgc tcagtccaag aggacatcaa ctccctgtgc ctgacggtgg tgcaacggct |
| 9541 | gatggagcgc atacagctcc catgggactc tgtgggcagg ctggaagtag gcactgagac |
| 9601 | catcattgac aagtccaaag ctgtcaaaac agtgctcatg gaactcttcc aggattcagg |
| 9661 | caatactgat attgagggca tagataccac caatgcctgc tacggtggta ctgcctccct |
| 9721 | cttcaatgct gccaactgga tggagtccag ttcctgggat ggtatgtacg gccacgaacc |
| 9781 | ttatgtaaga aaggtgctgg aattggaggc tgaatattac cagttttgct tttcagttcc |
| 9841 | ccaggtggct tcatctagtg aaggaaggac aatatattca cacagctgct gctatcatcc |
| 9901 | cacaataacc acttagactt atatagcttt acagttaggt agcatgttca catagccatt |
| 9961 | catttaattc ttacaacagc ctaggaagtg tgtattatac cagatttata gaagagaaca |
| 10021 | tggaagatct gatagcttac acatagtgag tggcagaggc aaaaatgcca aaccacatct |
| 10081 | gacatatttc ctattttacc gtacctgttt ctcttaaaca tgtcctaagt ctctgagaga |
| 10141 | ttggtgatgt tgaaagatgt atgcaagttt agatgttcgg gaaaaaaaca ccttcataga |
| 10201 | aacaggccca gaaaaccaca agatagactg tgagtatttc tactctttct cccttaggtg |
| 10261 | gctccttgca tattgctttt tgcttaacat attaacatta ccttgtatct tacttatatc |
| 10321 | ttctcccagt gctatatttg aggactaacc cctgttgtta cagcaagaaa tgattcaagg |
| 10381 | gaaacagtac agtatgagag cttgaagcca tagctctatc aataatcatt gataaattcc |
| 10441 | tgaacctctt tgagcctcag ggttatttgc ctatctgcct tgcttaactt ataagaggac |
| 10501 | tgaataaaat aattcataga aatgtgaaat tttcataaag atgtgaaaaa acagtatgtt |
| 10561 | ggcagtagtt aagacactct atatttacta agtttgaaac taggattaaa aaccttagaa |
| 10621 | accatgataa gcattaatta taaaattaat caaaaagcct taatattggc agagtcctca |
| 10681 | gagatcatct aattcaatat cttttgcttt agaaaaaaga ggtcaagagg agtgtaacag |
| 10741 | tttatctctg tacatgcagc aagaccgtgc aattacaaaa gttcattcca ggcttttcca |
| 10801 | actgccctac ctggctccat cattaacaat tccactgaca tgggatggtc cagtctacat |
| 10861 | catcaagtct gttcttaaag tgcctctcct acttgatact tgtattacta cctctctagt |
| 10921 | aacccctacc accattacca ccactgatat gtccaaccaa ttatttagtt gaggagtaga |
| 10981 | aatgaaaaat aaggggcatt caccagcctt taaccaaaaa tcaaagagcc tattcttgag |
| 11041 | agcattgtca gccttaagca tgccatttca aatgcgtaga ttcttctgag gggctgggta |
| 11101 | ttccacagat ggggttgcaa atgcatcttt taaaaaaatg tggtatctag gtataaaagt |
| 11161 | aaaaatttaa aaaacaagtt attgaaatgt gaatctttag tttgtattta aaacaaaaac |
| 11221 | agctaagctt gagcctggac actcggacta cataccctgc aggtgacagt aaccaccagg |
| 11281 | accagaggat gccagtgtga atgagaactc tgcttctgac ctagccagtc attcatctgg |
| 11341 | ggaccctcag gtgggaggga gtggctctga gactcaggga gttctgaatc actccagaga |
| 11401 | aaagtggagg ggatgaggaa agagaagagt atttctggct cagattggct gggagtcccc |
| 11461 | atgttttctt gtgttttttt ttttaaatga aaataattaa aatttatatt tggaaaaaaa |
| 11521 | catacacata cacaaaagta tataaagcaa agaaagactc ctcatttgac ctgttaccac |
| 11581 | ttcccaaaat ttaacactga tggtttatat gtattcttcc aatatttttt ctaagtacct |
| 11641 | gcaagtatac acatatctat tccattttaa acattgtaca aaatattcct catctcttag |
| 11701 | gtcttagagg taattctgta tcaacatatg taaggtctat ctgattcttt ttaaaaccac |
| 11761 | aatattcttg atggatatgc caaattttat ttaattaatc ccatattgat ggatatttag |
| 11821 | ttttttagca atgataaata aagttttaat gaacattgta caatagcttt gtatactttt |
| 11881 | ggcattgtat tgtaagaata aattcctaga agtggaatat caggataggt tgatttaaaa |
| 11941 | gtttgataaa atgtgccaaa ttcttctcca aaatgttgta ctaacttaca ttcctacaat |
| 12001 | gtatatatta tcaaactttc taatctttgt caatttaaca agtaaaatta taatgttttt |
| 12061 | gatttgcgtt tcttttacta taagaaatct tgaatatttc tatgttgttt attggccttt |
| 12121 | ttttattata tagcttgcct ttttttattt tttatttatt tattttttta gacagagtct |
| 12181 | cgatctgttg ccaggctgga gtgcagtggc ggtgatctca gctcactgca acctctgcct |
| 12241 | cccaggttca agcgattctt ctgtctcagc ctcccgagta gctgggacta caggacccca |
| 12301 | ccaccacacc cggctcattt tttgtatttt tagtagagat gggatttcac cgtgttagcc |
| 12361 | aggatagtct tgatctcctg acctcacaat cctcctgcct cggcctcccc aatcgctggg |
| 12421 | attacaggcg tgagccaccg tgcccggcct agcttgcctt tttaatgaaa cttttataaa |
| 12481 | tgaagataaa ttgatttttg ttgattgtaa gtattgtaaa tactccccca atttgtcttt |
| 12541 | tgactttgtt tctgatagaa ggctttgatt tttagataat caaatttact ggccttttcc |
| 12601 | taaatggatt ctaaatactt ttctatagtt tctaaagttt tcaaaatgtg tgcgtgtgtg |
| 12661 | tgcttatata caggtagaaa aaagtattgt tttcccttaa ttttatgtat ataaaaatta |
| 12721 | tatatactta aatatatatt tatatatatt aaatatacca atttacttat actaatatat |
| 12781 | ttatatatac taaatatata cttacattta tatatttata taaattattt gtatatttat |
| 12841 | atatatacac acacacacat gcacatagca ttggggaaga aaacaatact ttttcgttga |
| 12901 | tgttggagtt gggattgtta taattcttaa gagaaggtcc ctggatttca gtgaatttgg |
| 12961 | gttggagtcc tgactctgaa tccttaccct accatttatt agctatgtgg tttttgggca |
| 13021 | agtggcttaa attctttagc cctcagtttc ttcatctgta ggatggggat aactatatct |
| 13081 | gctacataga tttatcatga ggattaaatt atatagaaat gtggctccca aagcagtgct |
| 13141 | gtgggtgaat actgggagct tcctcacagg tcagaatact aaaattacta ccatatctca |
| 13201 | cccacaaact tgagtttttg ggacagtact tcttacagat gaaagtggaa cacataatag |
| 13261 | tcaagaccac aattatttat tgaatactag tctgattatc ataaagttag tgactacgga |
| 13321 | tcatttactc aatataaact attttcacaa tgaaagtagt gccacacaat tcaaggcacg |
| 13381 | tggttcagga tccagtcaga actgggtttg aatatcaaaa tccatattaa ctagctatgt |
| 13441 | gaccttacac tagttactca gtctctcagg aaggcaatgt cttcacttgt gaatgtggat |
| 13501 | gttacctacc tcattggatt gtttcaagaa ttgtttaagg ttaactagtg tcctactagt |
| 13561 | gttttaaatg ttagtttccc tccctgtcct ttaccttcta tgatttagga tataatttca |
| 13621 | ggatcatggt gtgctataag gagatgggta caaacccaaa cctgaattgt ctccaaaagt |
| 13681 | gcgaattaac acatttttca ctgaagtcag agacagaatt ctgaataaat gagcgtttta |
| 13741 | cagagtgtca ggacactaaa ttttgacttt acatttcaaa tgtatcatga attgcactag |
| 13801 | aacataagct ccacaggact gggatttttt attttgttta tcactctata tccaggacct |
| 13861 | agaattgtgc ctggtacaca gtaggcactc agtctactct agatttggta atgatggtaa |
| 13921 | atatttcttg tttctcttta caggtcgtta tgccatggtg gtctgtggag acattgccgt |
| 13981 | ctatcccagt ggtaatgctc gtcccacagg tggggccgga gctgtggcta tgctgattgg |
| 14041 | gcccaaggcc cctctggccc tggagcgagg tttgtagtaa tccattacca agaggctgtg |
| 14101 | catggcatag ccaagaacat agatcctaat cccacattgg cacacctgct actcagggct |
| 14161 | gaggtatgcg tttgaggatg gtattgcttg cctctaaaaa gggctggtct atggagcaga |
| 14221 | gggaggagag gagaaatggg agaggggaat ccgcgaggct tcctctcttg catcatcagg |
| 14281 | cattgggata acgatgcatg gaatgagtgg tgcagatgat ggtgaggaat cttagggaac |
| 14341 | tcttctggca attgaagatt aaaatatata actggatata aagtgaaagt ctttcctttg |
| 14401 | agactgttgg cttctattct aggttttgtt aagcccatgt aggtgaggaa agggaaatat |
| 14461 | acatctcatt tttgtaatac caacaacctg tccaactcct tttgaatatg caagggatgt |
| 14521 | tgaatgggct tgaacttggg catgggacac agataatgac cagaaacctc ctttatatgg |
| 14581 | ttctctcatc ttttgtgctc aaggtaggct gcattgtgta gtctctgaaa cactttgtgt |
| 14641 | gcctttccag ggctgagggg aacccatatg gagaatgtgt atgacttcta caaaccaaat |
| 14701 | ttggcctcgg agtacccaat agtggatggg aagctttcca tccagtgcta cttgcgggcc |
| 14761 | ttggatcgat gttacacatc ataccgtaaa aaaatccaga atcagtggaa gcaaggtatg |
| 14821 | agattcagag ggcagaaagt gggggctcta tttacatagg ccaagggttt gtacccaaag |
| 14881 | gccatgagat ggtcttttct ctcctgcctt gaaaataatg tcaagagaat tgtttcctgt |
| 14941 | cctctttctt acactcttcc ctgggtctat gctaaaatcc atttggaagt cattcaactt |
| 15001 | caggtgtaaa attgcttcta acttgagcta aataaaagaa agtaaataat ccagggcaag |
| 15061 | gcccccagtg tgaaaccaag ggatgtcagc cacctgagaa gatggtgtta agaggctggg |
| 15121 | cagtcacatt cgacagtggt tggcatttgt ttctggttaa gtcaggcatg gtttggctct |
| 15181 | tggtttgtgg tttaccatct tttaaagtct cacgttgaga aatcatacct atattttcta |
| 15241 | tatgctgaag tgttatcagt gatttttctc ttcgtgatgc tactgcaggt tgattttatt |
| 15301 | ttcaccttta gttttggaat ttccctcctg agaaatatgt actgctttca taagcagaaa |
| 15361 | ataagcaaat aaatcttcct tttaaaatac agaaaagcag ggagtggtgg ctcacgcctg |
| 15421 | taatcccagc accttgggaa gctgaggcag gaggattgct tgaacccagg aatttgagac |
| 15481 | caatgtgggc aacaaagcaa gaccctgtct ctaaaaaaaa aaagtacaaa agttagccag |
| 15541 | gcatggtggc ataagcctgt agtcccagct actcagaagg ctgagatggg ggaaattgct |
| 15601 | tgagaccagg agcccatgca gtaagctatg atcaagcaac tgcctccagc ctggactaca |
| 15661 | gagtgaaaca aaccctgtct ctaaaaacat ataaataaat aaaaataaaa tacagttaaa |
| 15721 | cctactttaa agacataaat agtattcttg cctgctcagg catgcccaga tgggcatccg |
| 15781 | caaaagacag attgcagtgt gggagaaggc atggatgcct tgggggtgtc ataaagagct |
| 15841 | acctcttgtc cctttctact gcagtgggtg ggacaccacc tgccagaggt gaacctcatg |
| 15901 | ggcaagaagt tgctttgggc ctctctgcct cagtctgtct tctgtaattg gttatttgct |
| 15961 | cctaactcct ctgaattctt gtggcattta aattttactc cttatttgca tatgtaaggt |
| 16021 | gacagatgct gctttggatc ccagcactaa aatgtaatat ttcctaaggg cagagattgc |
| 16081 | attgccctct tcttcagagt gagagagaca gtctgtagag tagagtcaga gacatctgaa |
| 16141 | cctgaatcca aagccagcct tttcaaagtt ggacagatga caatgttttg tagaccggtt |
| 16201 | cctcctctgg caaatgaaga aaattatata acacaaggtt gatttgagcc aagtatcata |
| 16261 | gaggctggta atagtagata caaaggcttt gtttctttcc cttctttcct tattcgtaga |
| 16321 | gattgcttag taagtgcatg taaaatgaat aaataaagct catatgtgtt tgcaggaggt |
| 16381 | gggaagtagt tccctgggag gcctggagaa actcggcaca gttaaatctc agggaggata |
| 16441 | tctaaatggc tcgcccctca tgccccatcc ttgccttcac gcttcctctt ccagctggca |
| 16501 | gcgatcgacc cttcaccctt gacgatttac agtacatgat ctttcataca cccttttgca |
| 16561 | agatggtcca gaagtctctg gctcgcctga tgttcaatga cttcctgtca gccagcagtg |
| 16621 | acacacaaac cagcttatat aaggggctgg aggctttcgg gtgagttctc ttcttgggga |
| 16681 | gcctagaggc tggtgaggtg tgagcaagaa ggaggcttct tcatgcctta agtctagacc |
| 16741 | accagcaccc ctgtggggga caaatggcaa tcctccagca gaacaggaac aatcccaggt |
| 16801 | ccttccacgg ggtagtgggt tattgtctgg gtagggccct ccatgagtta ttgcagggaa |
| 16861 | acatggggga tttggcagca ctgcaggatc aaggggcagt aagaaactac agaggataaa |
| 16921 | gaaagaaaga gagaaaggga gaaagagagg aagggagaaa gagagtagct aaatcattca |
| 16981 | gtcaataaac attttctgaa catgttatgt gctagacatc gtattaacct ctcaggatac |
| 17041 | taaaatgaat gtgactccat ggtccctgcc ctagagcatc tcacagccta tacagacaca |
| 17101 | aacacacaga agcaaatgat cacactacag ggtagcaatt tgagaagtgt caggtcccat |
| 17161 | tctcatttgc cattgtctta attcatgtcc tgcttttgct tttctcccat ctataaaatg |
| 17221 | gggatgttcc agctcatccc cttagatgtg aaaaagcaga aagaatgctg tttattgatt |
| 17281 | cactacacta atacactaat atttacaaag aaatgtcttc aatacagttt ccactgggaa |
| 17341 | aggaatcttt ccctttcttc ttggtacctg tttatttcaa attttggtca attttatcaa |
| 17401 | cagtagaata ggctaccaag tgtagcccct gttactaact agtactccta accctgccac |
| 17461 | taactaaaac atcaaaatta gcacaaacac tgcttgtaag accagcccta tcgaaacaaa |
| 17521 | aagtataaca tataccaaag atactagctt aatatcttta atatataaag atattatcaa |
| 17581 | taataaaata aataccctaa tagaaaaatg agcaaaggat atgaacagaa aattttctca |
| 17641 | aagaagacat gtatatgatc acattttaaa tatgcatatt cattaataaa aagtttactc |
| 17701 | aagttaccat tttctctaga tagtcttttt aaaatgtgaa tacccagaat tgtcaagcct |
| 17761 | gtgggaaaat gggcagtagt ggatgcgtaa atggatcagg tgttttgagg agtaacagga |
| 17821 | gagcatgaag ccaaagcctt aaagatgtgc aaactttggg ctcagtaatc ccatgtgttt |
| 17881 | aaaagaacac ctacctattc gctgtagtgt tttaactagt gaggaacagg agcaggaaga |
| 17941 | tgggttaaag tgcggtacat cctgtcatgg accattcttc agcctttaca aataatgtta |
| 18001 | tagaatgtca tggaaaaaaa atatatatat atatacacac acacactaag ttaagaaagt |
| 18061 | atgctaacca caacacatag catgattttc tttctaattt tctagtaagc tctataaaat |
| 18121 | tagggatgga ttccaccaga aaaataagcc ctaagtactc tctctgaatg gtaaggccat |
| 18181 | tagtggtatg ttctcctctg tactgttctg tatttccaaa tattgtagga aaaacatgcg |
| 18241 | ccccaaagtc ctctccagaa gctgttactt ttcccccttg ctccctgcct cccgtcccct |
| 18301 | ggcctctcac atggctacct ctggctacct cacagggggc taaagctgga agacacctac |
| 18361 | accaacaagg acctggataa agcacttcta aaggcctctc aggacatgtt cgacaagaaa |
| 18421 | accaaggctt ccctttacct ctccactcac aatgggaaca tgtacacctc atccctgtac |
| 18481 | gggtgcctgg cctcgcttct gtcccagtga gtactgcatc tggctccatg tcctccatgc |
| 18541 | acaccctcag cctccgcccc cgtgggctgc agggtcaaca aagttgggtt tctcttttgg |
| 18601 | ctcagaaatt taaaagaaag gaaggggcct ggtgtagtgg ctcatgcctg taatctcagc |
| 18661 | atttggggag gtttaggcgg gcagatcgcc tgaacctagg agttcgagac ccgcctgggc |
| 18721 | aacgtggtga aacctcatct ctacaaaaat tagctgagca tggttgtgtg cacgtgtggt |
| 18781 | cccagctgct cgggaggctg aagtgggagg atggtgtgag cccaggagtg gaaggttgca |
| 18841 | gtgagccatg attgtgtcat tggactccaa cctggatgac agaatgagat cctgtcataa |
| 18901 | ataaataaat aaatataaaa gaaaggaaag gagggagaag gcaggaaaag gaaggaagat |
| 18961 | gaaagaaact cgtaccaaag gtgtatgtat aggcagattt acagtctgta tcagacagtg |
| 19021 | gtctccaaag tgaagtacat gatgtcaagg gatgggcaag atctgtttgg gcacatcaag |
| 19081 | aaaacagtag ctttggtatg catatttttg tctcatttat ttaaaatctc tatacttagt |
| 19141 | agagcatggt ggttaaatgg gtctgacttt agagcccaca acctgggttc aaattttttt |
| 19201 | aaccaattat tagggttgac tttggataat acttaacctc aatgcacctc accttcccca |
| 19261 | actgtagcat gtgtgcaatc acaatacctg tgttctactg tttttatgag cattaagtat |
| 19321 | ctaaaacaat taaaatagca gtgcttagca ggtgctcaaa tgttggatgt tatttctatt |
| 19381 | cattttctgt tttgtgggtt ttataaggaa gtactgcatc taacataaga aagggctcat |
| 19441 | gaagtggctc atgcctataa tcctagcact ttggaaggct caggcaggag gatctcttga |
| 19501 | gctcaggagt ttgagaccag ccttgggaac agagggaggc cccatctcta caaaattttt |
| 19561 | ttaaacaatt agccatggat gttcacggtg gctcatgcct gtaataccaa cactttggga |
| 19621 | ggccaaggtg ggaagatcac ctgaggtcag gagtttgaga acagcctggc caacatggca |
| 19681 | aaaccccttc tctactaaaa atacaaacat cagctgggca tggtggtacg tgcctgtagt |
| 19741 | cccagcaact cgagaggctg aggcatgaga attgcttgaa cccgggaggc agaagttgca |
| 19801 | gtgagctgag atcgagctac tgcactatag cttgggtgac agagtgagac tctgtctcaa |
| 19861 | aaaaaaaaaa aaaattagct gggtgtggca gcttgcacct gtagtcccag ctactcagga |
| 19921 | tcctgaatcc tgaggtggga ggatcacttg agcccaggag gtaaaggctg cagtgagcca |
| 19981 | tgatcacgcc actgcattcc gggcactcca ggctgggcaa cagagcaaga ctctgccaaa |
| 20041 | aaaagaaaaa aaaaacgggc aggaaaaagt gcttatgggt gaacttgatc aaattattac |
| 20101 | tcacagggga tgatcaaaaa gttatgactg ctgaaccatt accaatcaac atgggagcct |
| 20161 | gaagggtgag tccagtggtc tgatctccat ctggagacac cttcagaatg cactgaattt |
| 20221 | accctgtcct catgagaggg gagaagctct atgtacacca aaaattatct tgtgttttct |
| 20281 | ctgccttata tatcttggat attagctgct ttccttttgg caaggtttcc tacacaaagg |
| 20341 | cctgtccctg gggtctacca gaagtccctc tttatgtagg gtgcctggaa cccatttcta |
| 20401 | gttgcatgag gtagacaggg agaagatcgg gatgataggc tgttgttcta tttgaagtgc |
| 20461 | agaatataat atatatatac atatatgtgt gtgtgtgtgt gtgtgtgtgt gtgtgtgtgt |
| 20521 | gttttatttg atttctttcc ccacagccac tctgcccaag aactggctgg ctccaggatt |
| 20581 | ggtgccttct cttatggctc tggtttagca gcaagtttct tttcatttcg agtatcccag |
| 20641 | gatgctgctc caggtgagtg tcatctttct agtaggcctt cctgacaaga ttcatctggt |
| 20701 | agaataacca tcttcttccc caccattact gaggctgcca tcttgacaga gttacgttat |
| 20761 | tattaatagc aaagtaaatc actgaaggga tttaagcatg gagtaagttt gtttaattta |
| 20821 | tgtgtttaaa gcacttattt ggctactact tagagactag attgaaaagg aacaaagctg |
| 20881 | gatatgggga aaccacttag attgttccag taactagttc aggcaagagg taatggtggt |
| 20941 | ttgattgcaa ctgattaaag agaagttgat ggatttgaga tacctaataa gaatttattg |
| 21001 | attattttgt gattgatgtg attaaggaca tgcatttaag tactatgtgg catacacctt |
| 21061 | gaccaaatca gtgtgtctgc ctgcatgttt tgctaacaag tatgcttgct tatcatttct |
| 21121 | tggtattcta agccacacac accacacgtt cctccagggt gtaacctccc acagaacctg |
| 21181 | gctctctgtt gaactcgtga ttggcaatag tgataatgac aatgaaaaag gtgtaacaat |
| 21241 | cttgcttttg cttcccaggc tctcccctgg acaagttggt gtccagcaca tcagacctgc |
| 21301 | caaaacgcct agcctcccga aagtgtgtgt ctcctgagga gttcacagaa ataatgaacc |
| 21361 | aaagagagca attctaccat aagggtaaga aaaaagtcag gaagagagga agagagaccc |
| 21421 | cattccagta gctgggagcc agggatttct ttggaaatct agaatttagt agtccagggt |
| 21481 | caagactttt acgagatatg gttgggagaa gatttgctag aagatctgtt gtccaaaggg |
| 21541 | gcaagaagtg ggtggggaaa cagaagatag agttgggaag agggaggcag gatgcagctt |
| 21601 | cccagtatag aatatagcta aacacccaga atgtgtagtc ccatggaagc cagaagtata |
| 21661 | gtctttgaaa ataccatctg caacagttga aagagtacag actttagagc tagatatcca |
| 21721 | aatctaaccc tgagctgtgc cactcactag ctgtttatct ttggaaaaat ggttgaactt |
| 21781 | ttctcagttg tcttatttct aaaatcatac cgattttgca ggatttccaa acaaattaaa |
| 21841 | tgaattactc tatataaata tgttatcgac aaatattact gtcccctcca aattgccctc |
| 21901 | tttctccacc aaacataaaa acaaaaaaca aaatattgct ccaaaagcaa caaatgaaag |
| 21961 | gaaaatgaaa cccaaaggta atactagagt gattagttgg tggttttaaa accatagtaa |
| 22021 | tacacagttt taccatgatt tctacaggtt ttatatatat tctcaagcaa aacttgggat |
| 22081 | gcatgttgtt ttgcagcatg gtctcaaaag gagacagaat atacggaatt ggaaatgttc |
| 22141 | cagaaaacct agacctagtg gtcattgatc tcttctggac cagtggatat gttatagcaa |
| 22201 | agaaagacaa tgaaaataaa aatggagcag ggcacagtgg ctcacgcctg taatgctagt |
| 22261 | cctttgggag gcagaggcag gtggatcact tgaggccagg agtttgagac cagcctggcc |
| 22321 | aacatggtga aaacccatct ctactaaaaa tataaaaatt ataaaaatat gaatataata |
| 22381 | aaaaaaataa aattatgtaa aaattagccg agtgtggtgg cacacacctc taatctcagc |
| 22441 | tactcaggag gctgaggaga attacttgaa cccaggaggc agaggatgca gtgaactgag |
| 22501 | atcacaccac cacactctag cctgggtgac acagaaagac tctgtctcaa aacaaaaaaa |
| 22561 | aaaaaaagaa gaaaagaaaa ataggacctc tgagacaaac gttaacggac aaagcactga |
| 22621 | aatactgcaa tgaatcagaa ccagaaaatt tagagtttag aaggacgtgt ctgttaggaa |
| 22681 | acaggaagct gggaattacg tctcaaagta ggaactattg gcaaaaggat gggatgaaga |
| 22741 | tttcaatgga ggaaggctat gtttactgta ggaaaatgtt gtactcttat aataaaagtc |
| 22801 | ttaatagact tttattaagg ccttaagtgc tagattcaag atggctgccc ctcttgttct |
| 22861 | gtgggtccag tgttctattt ggtggactaa gggtgacctt gcagcccctt acagcccagc |
| 22921 | caagagagct tcactgtgaa ggggcagaca tcttcattac tattttctct tccaaaaact |
| 22981 | catataactc tttgtgagta ctgcctcttc tcctcattcc acagtgaatt tctccccacc |
| 23041 | tggtgacaca aacagccttt tcccaggtac ttggtacctg gagcgagtgg acgagcagca |
| 23101 | tcgccgaaag tatgcccggc gtcccgtcta aaggtggtga gtgagagttt gcagagttgg |
| 23161 | tggcataaaa ccctaatgtc ttcctctgag taacaacaca gagagagaag gtggggacag |
| 23221 | gtgcagggag aagaaagttt aatggaagag gattggggtg acaggagaaa tgggagaatt |
| 23281 | atctgtggaa tttttaaaag gaaaagcaag tattcagaat aggaatcttg tagtttggga |
| 23341 | acattaacca ggccagggag ggttcacagc tttcaaacta atcagaagtg gggatttgta |
| 23401 | ccataaagac caattaaaac tcttggggct ctttgccttg gaaaggcaaa agctggggga |
| 23461 | gaaacatgtt ctgaaatctt gaatgtgaaa aataggagct ggatttgttt acctgatctg |
| 23521 | ctgaagatag gaagctctcc tagaagcttg acagattagc attcagagca tccgttgagt |
| 23581 | gaacaggctg tgaacctgaa cctatagaaa tcattactcc agggggatga gatcaacaga |
| 23641 | tctgatgagc aacagaacaa ccaagatgaa cagccccaaa acctcagaaa tggtacacac |
| 23701 | caatgtgtgg gagacagatt cataaggaat ggggcggttg aagattctgt taaagccaga |
| 23761 | tacttctgct ggagggagtt ttaggctaag ggtcatgtaa caattcttat atcatgggat |
| 23821 | tccttctggg gagaagcaat gaggttcagg aaattcgtgg acacaaggat agggagaaga |
| 23881 | gagcaaggtg aaagaggatt gcggtgacag gagaaatggg agatattctt tatgatcgtt |
| 23941 | tttaaaggaa agcaaacatt caaaaataag aatcttatat gaacccaggt agctgccttc |
| 24001 | agttgaccaa ataggtagga taagcagaat gatagagtga gaagagattt attttacaac |
| 24061 | ccataaattt taattagtgc agtctccatg ctcaagtttt taagattttc ccctcctttt |
| 24121 | ggtagatgga gagggaagaa gaaaaaggtg tgccgaggca gggaaggagc agaggaaggg |
| 24181 | aaggaagaag tcagtgggtg gcagagatgc acagatacag ccacctgaga ggaagcagag |
| 24241 | gtgcgggtgg aggggccctg ggttcattcc ttaccgctgg gatattggca ggtgctaggc |
| 24301 | tgttgcagcc cagatgttgt tagggctagg agaggtggac aagtgggctg agggccgcag |
| 24361 | gatgcctttg agaggacgag ctcagttagc agccctgaag actgtggtac tgcccgggag |
| 24421 | cctgtgtgca tgttggaaat acggttctta agggcaggtc agtagcaaag aggggctgtt |
| 24481 | aaatgtgtca acttagttca ttcatcagaa gaagagtggg agaaataggg agggaggggg |
| 24541 | gaaagggaga gagagaggtt ggggagagag tcagcgggag ggggagagag aaagagaaat |
| 24601 | ttggaatttt taaaggagaa tttccacgtc agcctccctc cctctcatgg tagacaagct |
| 24661 | tcttgcaagt gcttaggcag aattatacct gaaaaaaaaa gctggaactc ttgacctttt |
| 24721 | ctcatgttga ttattaatat gagcagtgaa cttccaacaa tgagatttta gcagaaatga |
| 24781 | agggctgctg tcagtgcagt gctcatggtg gagctctaca ggtctctgca gcgccctagc |
| 24841 | ctgcctctcc tgctctccta tcacaggcag atgtgcgacg gggaccctgc ctacccccag |
| 24901 | ccttggctcc agtagcattg ggcacagatc cctcaggtgt ccaggcttgg cacagggtgc |
| 24961 | atagtgggag caccctcagg atgcagttag gggagcccct ctgcacagcc acacctcggg |
| 25021 | caagaagcag gtactggggg cagggtgccc aagaggagac ccatgattga atgacttttt |
| 25081 | gtttatttaa gttctgcaga tccatggaaa gcttcctggg aaacgtatgc tagcagagct |
| 25141 | tctccccgtg aatcatattt ttaagatccc actcttagct ggtaaatgaa tttgaatcga |
| 25201 | catagtagcc ccataagcat cagccctgta gagtgaggag ccatctctag cgggcccttc |
| 25261 | attcctctcc atgctgcaat cactgtcctg ggcttatggt gctatggact aggggtcctt |
| 25321 | tgtgaaagag caagatggag caatggagag aagacctctt cctgaatcac tggactccag |
| 25381 | aaatgtgcat gcagatcagc tgttgccttc aagatccaga taaactttcc tgtcatgtgt |
| 25441 | tagaacttta ttattattaa tattgttaaa cttctgtgct gttcctgtga atctccaaat |
| 25501 | tttgtacctt gttctaagct aatatatagc aattaaaaag agagaaagag gaaatgattc |
| 25561 | ctgcgtttct tggaacccag aatacaaacc cagcctaaca tgcagcaagc ctgctagacc |
| 25621 | ttgtgggtca gagggctggg tccttgcctc acaggctgcc tctgtcccct tgcaattcca |
| 25681 | ttctatttct gccacatgcc aagtgctatg acaggtacaa ggcaaataag aacggtagaa |
| 25741 | cacagcttcc cccagcccac ttccctgttc taaagacacc acatagacag agagcagcag |
| 25801 | acaggggcca gcaggagctg tagttcagat cttcttggtc attccttgcc gctgttattt |
| 25861 | gaacaaataa acacagcgca aaggttaaca agtttttgcc ttctatagcc aaaaataaaa |
| 25921 | aaataaataa attttgatgc ctggcaggaa attattccat tacaggatct ttcccccttg |
| 25981 | ggggagggca ctgcttcttc tagggtcctc ttataaaata gcaatggttc aggcagatgg |
| 26041 | ggattgagct gaggacggga gtgggaggag agggaaagta tcagggtgtt gtcatcactt |
| 26101 | ccttttagaa agtttcctca gtcaccccca tgaggaaagg gcaccttgga aaagagagag |
| 26161 | gatgctttcc attggcgggg agcagagctg gtgggggcag gggaggagga ggggaggagg |
| 26221 | aggaggagga gaagcagggg aggcttaagg ctcccttaag cctcagggag cgcttaagaa |
| 26281 | tggccccaca ggaatgagaa gctgggtctg ttcccttcac tgttttgctc aaggctgttc |
| 26341 | atgtcacaac aaatcccaga taagccccaa tttgctcaga gaatccagca ttagctgact |
| 26401 | gccttcccag gcctctctca aggtgcctgc aaaactctac tcatcacacc agctgcagcc |
| 26461 | gctgcttagc agcccctctt tgctaccctc ttgctgcctg cacctcctca gcaagatgtt |
| 26521 | taggggccct caacctggtt ggcatcccta gcagaacaac atgtgccttt cggtatctgt |
| 26581 | gtgcagggga gaaaacccag cactaacctt agctctggag acaagaggcc tcgggcctgg |
| 26641 | ccttctatcc acacagaagc tcactgtgca gtgttggtgc tgaaactctc tccatcagcc |
| 26701 | tcagtcagcc tcagcaacca gaacttccca tacttcctgc atcagaggcc aggcctgtct |
| 26761 | ccactaggga ggcatttgag cacaaatgga atgatattaa acattcgaca accaggttgt |
| 26821 | caagggctga ccaattgaat ggacactgcc cacagcccac acaccagctg ggcatcagca |
| 26881 | ctggctccct ccaacttcct tattcaccaa cttttatact gagcccgagg ccttcctctg |
| 26941 | gcagctctgg gacactgatg cctgcctgct ctgaacaaag ccctctcccc catgtaaggt |
| 27001 | cagcacacga gggaatgagt tgccaatggc tcagtcaaca ttttcaccct aaagtctaca |
| 27061 | gataccatac aaataaagac tttccctgtg ggcaaaaatt cacacagggt gacctagggc |
| 27121 | aggagagagg acggcagatt gggcaagtgt tgggctatga tacactcatt caaacgggaa |
| 27181 | tactcaacat gtgatgttaa aactgatgca aaagatggcc ccgccactga ccatgagaca |
| 27241 | agcccaagct ctagggggac acactgatca caacttcagg agtcagcaca ttgaggcaga |
| 27301 | ttctgtgcgt ggcccagctt ttgccctgcc tccaccctga gctcacagcc agccttctgc |
| 27361 | tgtgtgtgca caagaatgaa cttctactct aaaggggcag tgaagagatg ccacatgcca |
| 27421 | caaagaacat gagggagtcc atggcaccct ccctgtagcc ctagctggat ttttcaaaaa |
| 27481 | tttcattgta tatatttgag ggatagaaca tgacgttgta agatatatat atgtagtaaa |
| 27541 | atggttactg taacggaaca aattaacata ttcattattt tacaaagtta cccatccccc |
| 27601 | gccaccatgg caagagcagc tgcaatctac taatttagga aaaatcctca gtacaataca |
| 27661 | ctgttattaa ctatagtcct caggttgtac atcagatctt ttgacttact caccctatgt |
| 27721 | attttctact ttacattctt tgacctgtat ctccctagac acccccctca actacttttc |
| 27781 | tagttcctat gtcaatatat ttgacctctt ttttgggggg ggattccaca tataaatgag |
| 27841 | taagtgcaat aattttcttt ttgtgtctgg cctatttact tagtcatcag ggaaatgcaa |
| 27901 | atcaaaacca cggtgagata ccacctcaca cctgtta |
| // | |

What is claimed is:

1. A gene delivery composition comprising a gene delivery vehicle and a heterologous genome wherein the gene delivery vehicle houses or encapsulates the heterologous genome and wherein the heterologous genome comprises nucleic acid sequence at least 80%, 90% or 95% identical to SEQ. ID NO.:1.

2. The gene delivery composition of claim 1 wherein the heterologous genome encodes human 3-hydroxy-3-methylglutaryl-CoA synthase 2 (mitochondrial) (HMGCS2) or its various isoforms.

3. The gene delivery composition of claim 1 wherein the heterologous genome further comprises a 5′ primer site and a 3′ primer site flanking the nucleic acid sequence.

4. The gene delivery composition of claim 1 wherein the heterologous genome encodes HMGCS2 enzyme or any of its functionally homologous forms.

5. The gene delivery composition of claim 2 wherein the 5′ primer site comprises nucleotide sequence at least 80%, 90% or 95% identical to the nucleotide sequence of SEQ ID NO:2 and the 3′ primer site comprises nucleotide sequence at least 80%, 90% or 95% identical to the nucleotide sequence of SEQ ID NO:3.

6. The gene delivery composition of claim 1 wherein the gene delivery vehicle comprises a liposome or polymeric nanoparticle.

7. The gene delivery composition of claim 1 wherein the gene delivery vehicle comprises a recombinant adeno-associated virus (rAAV).

8. The gene delivery composition of claim 7 wherein the rAAV comprises an AAV9 capsid.

9. A method of treatment for cardiac ischemia comprising the step of providing a therapeutically effective amount of HMGCS2 to a patient.

10. The method of claim 9 wherein the step of providing a therapeutically effective amount of HMGCS2 to the patient comprises the step of upregulating the expression of HMGCS2 in the patient's cardiomyocyte (CM).

11. The method of claim 10 wherein the step of upregulating the expression of HMGCS2 in the patient's CM comprises the step of administration of a therapeutically effective amount of the composition of claim 1 to the patient's heart.

12. The method of claim 11, wherein step of administration of a therapeutically effective amount of the composition of claim 7 to the heart comprises administration of between about 10⁷-10¹⁸, about 10¹¹-10¹⁷ or about 10¹²-10¹³ of the rAAV of claim 7.

13. The method of claim 9 wherein the step of providing a therapeutic effective amount of HMGCS2 to the patient is performed before the cardiac ischemia.

14. The method of claim 9 wherein the step of providing a therapeutic effective amount of HMGCS2 to the patient is performed after the occurrence of cardiac ischemia.

15. The method of claim 14 wherein the step of providing a therapeutic effective amount of HMGCS2 to the patient is performed 1 day, 2 days, 5, days, 10 days, 20 days or 30 after the occurrence cardiac ischemia.

16. A method of treatment for cardiac ischemia comprising the step inducing a metabolic switch of adult cardiomyocyte (CM) using HMGCS2.
